(19) United States
(12) Patent Application Publication
(10) Pub. No.: US 2014/0186877 A1
(43) Pub. Date: Jul. 3, 2014

(54) COMPOSITIONS AND METHODS FOR THE BIOSYNTHESIS OF 1-ALKENES IN ENGINEERED MICROORGANISMS

(71) Applicants: Nikos Basil Reppas, Cambridge, MA (US); Christian Perry Ridley, Acton, MA (US); Amy Dearborn, Lowell, MA (US)

(72) Inventors: Nikos Basil Reppas, Cambridge, MA (US); Christian Perry Ridley, Acton, MA (US); Amy Dearborn, Lowell, MA (US)

(73) Assignee: Joule Unlimited Technologies, Inc., Bedford, MA (US)

(21) Appl. No.: 14/240,118

(22) PCT Filed: Aug. 22, 2012
(86) PCT No.: PCT/US12/51925

§ 371 Date: Feb. 21, 2014

Related U.S. Application Data
(60) Provisional application No. 61/526,178, filed on Aug. 22, 2011.

(51) Int. Cl. C12P 5/00

(52) U.S. Cl. CPC C12P 5/002
USPC 435/29; 435/167; 435/257.2; 536/23.2; 435/320.1; 435/188; 435/254.2; 435/258.1; 435/254.11

(57) ABSTRACT

Various 1-alkenes, including 1-nonadecene and 1-octadecene, are synthesized by the engineered microorganisms and methods of the invention. In certain embodiments, the microorganisms comprise a recombinant alpha-olefin-associated enzyme. This enzyme may be expressed in combination with a recombinant alkene synthase pathway-related gene. The engineered microorganisms may be photosynthetic microorganisms such as cyanobacteria.

### CROSS-REFERENCE TO RELATED APPLICATIONS

This application claims the benefit of U.S. Provisional Patent Application No. 61/526,178, filed Aug. 22, 2011, the disclosure of which is incorporated herein by reference.

### SEQUENCE LISTING

The instant application contains a Sequence Listing which has been submitted in ASCII format via EFS-Web and is hereby incorporated by reference in its entirety. Said ASCII copy, created on Aug. 22, 2012, is named 21328PCT_CRF_sequencelisting.txt and is 123,383 bytes in size.

### FIELD OF THE INVENTION

This invention generally relates to genes useful in producing carbon-based products of interest in host cells. The invention also relates to methods for producing fuels and chemicals through engineering metabolic pathways in photosynthetic and non-photosynthetic organisms.

### BACKGROUND OF THE INVENTION

Unsaturated linear hydrocarbons such as α-olefins or 1-alkenes are an industrially important group of molecules which can serve as lubricants and surfactants in addition to being used in fuels. The biosynthesis of organic chemicals can provide an efficient alternative to chemical synthesis. Thus, a need exists for microbial strains which can make increased yields of hydrocarbons, particularly terminal alkenes.

### SUMMARY OF THE INVENTION

The invention relates to a metabolic system and methods employing such systems in the production of fuels and chemicals. Various microorganisms are genetically engineered to increase the production of alkenes (also referred to as olefins), particularly 1-alkenes, including 1-nonadecene and 1-octadecene.

In one embodiment, a method for the biosynthetic production of 1-alkenes is provided, comprising culturing an engineered microorganism in a culture medium, wherein the engineered microorganism comprises a recombinant alpha-olefin associated (Aoa) enzyme and produces 1-alkenes, and wherein the amount of the 1-alkenes produced by the engineered microorganism is greater than the amount that would be produced by an otherwise identical microorganism, cultured under identical conditions, but lacking said recombinant Aoa enzyme. In another embodiment, the engineered microorganism further comprises a recombinant 1-alkene synthase. In one embodiment, the microorganism is a cyanobacterium. In yet another embodiment, the cyanobacterium is a Synechococcus species.

In one aspect, the engineered microorganism comprises a recombinant 1-alkene synthase at least 90% identical to YP_{—}001734428 from Synechococcus sp. PCC 7002. In another aspect, the engineered microorganism comprises a recombinant 1-alkene synthase at least 90% identical to SEQ ID NO: 5. In still another aspect, the engineered microorganism comprises a recombinant 1-alkene synthase comprising SEQ ID NO: 5. In yet another aspect, the engineered microorganism comprises a recombinant 1-alkene synthase consisting of SEQ ID NO: 5.

In another aspect, the engineered microorganism comprises a recombinant 1-alkene synthase encoded by a gene at least 90% identical to a nucleotide sequence selected from the group consisting of: SEQ ID NO: 2 and SEQ ID NO: 4. In still another aspect, the engineered microorganism comprises a recombinant 1-alkene synthase encoded by a gene comprising a nucleotide sequence selected from the group consisting of: SEQ ID NO: 2 and SEQ ID NO: 4. In yet another aspect, the engineered microorganism comprises a recombinant 1-alkene synthase encoded by a gene consisting of a nucleotide sequence selected from the group consisting of: SEQ ID NO: 2 and SEQ ID NO: 4.

In one embodiment, the recombinant Aoa enzyme is at least 90% identical to the amino acid sequence given by accession number YP_{—}0001735499 from Synechococcus sp. PCC 7002. In another embodiment, the recombinant Aoa enzyme is at least 90% identical to SEQ ID NO: 7. In yet another embodiment, the recombinant Aoa enzyme comprises SEQ ID NO: 7. In still another embodiment, the recombinant Aoa enzyme consists of SEQ ID NO: 7. In one aspect, the recombinant Aoa enzyme is encoded by a recombinant gene at least 90% identical to SEQ ID NO: 6. In another aspect, the recombinant Aoa enzyme is encoded by a recombinant gene comprising SEQ ID NO: 6. In still another aspect, the recombinant Aoa enzyme is encoded by a recombinant gene consisting of SEQ ID NO: 6.

In yet another aspect, the recombinant Aoa enzyme is at least 90% identical to an amino acid sequence selected from the group consisting of: YP_{—}0001735499 from Synechococcus sp. PCC 7002; YP_{—}003887108.1 from Cyanothece sp. PCC 7822; YP_{—}002377175 from Cyanothece sp. PCC 7424; ZP_{—}08425909.1 from Lyngbya majuscule 3L; ZP_{—}08432358 from Lyngbya majuscule 3L; and YP_{—}003265309 from Haliangium ochraceum DSM 14365. In still another aspect, the recombinant Aoa enzyme comprises an amino acid sequence selected from the group consisting of: SEQ ID NO:7, SEQ ID NO:9, SEQ ID NO:11, SEQ ID NO:13, SEQ ID NO:15, SEQ ID NO:17, and a homolog or analog thereof, wherein a recombinant Aoa enzyme homolog or analog is a protein whose BLAST alignment covers >90% length of SEQ ID NO:7, SEQ ID NO:9, SEQ ID NO:11, SEQ ID NO:13, SEQ ID NO:15, SEQ ID NO:17 and has >50% identity with SEQ ID NO:7, SEQ ID NO:9, SEQ ID NO:11, SEQ ID NO:13, SEQ ID NO:15, SEQ ID NO:17 when optimally aligned using the parameters provided herein. In a related aspect, the Aoa enzyme is encoded by an aoa gene selected from: SEQ ID NO:6, SEQ ID NO:8, SEQ ID NO:10, SEQ ID NO:12, SEQ ID NO:14, SEQ ID NO:16, and a homolog or analog thereof, wherein an aoa gene homolog or analog is a nucleic acid sequence whose BLAST alignment covers >90% length of SEQ ID NO:6, SEQ ID NO:8, SEQ ID NO:10, SEQ ID NO:12, SEQ ID NO:14, or SEQ ID NO:16 and has >50% identity with SEQ ID NO:6, SEQ ID NO:8, SEQ ID NO:10, SEQ ID NO:12, SEQ ID NO:14, or SEQ ID NO:16 when optimally aligned using the parameters provided herein.

In one embodiment, the recombinant Aoa enzyme is an endogenous Aoa enzyme expressed, at least in part, from a promoter other than its native promoter. In another embodiment, the recombinant Aoa enzyme is a heterologous Aoa enzyme. In still another embodiment, the recombinant Aoa enzyme is expressed from a heterologous promoter. In yet another embodiment, the heterologous promoter is tsr2142. In still another embodiment, the promoter is at least 90% identical to SEQ ID NO: 20. In a related embodiment, the Aoa enzyme is endogenous to said microorganism.

In one aspect, the engineered microorganism is a photosynthetic microorganism, and exposing the engineered microorganism to light and an inorganic carbon source results in the production of 1-alkenes by the microorganism. In another aspect, the engineered microorganism is a cyanobacterium. In yet another aspect, the engineered cyanobacterium is an engineered Synechococcus species. In still another aspect, the 1-alkenes produced by the microorganism is 1-heptadecene, 1-nonadecene and 1-octadecene, or 1,x-nonadecadiene. In still another aspect, the invention further comprises isolating the 1-alkenes from the microorganism or the culture medium.

In one embodiment, the 1-alkenes are selected from the group consisting of: 1-tridecene, 1-tetradecene, 1-pentadecene, 1-hexadecene, 1-heptadecene, 1-octadecene, 1-nonadecene and 1-octadecene, and 1,x-nonadecadiene. In another embodiment, the 1,x-nonadecadiene comprises 1,12-(cis)-nonadecadiene. In yet another embodiment, the method further comprises isolating the 1-alkenes from the cyanobacterium or the culture medium. In one embodiment, the amount of 1-alkenes produced by the engineered microorganism is at least four times greater than the amount that would be produced by an otherwise identical microorganism, cultured under identical conditions, but lacking the recombinant alpha-olefin-associated enzyme. In another embodiment, the rate of production of the 1-alkenes by the engineered microorganism is greater than 0.05, 0.06, 0.07, 0.08, 0.09, 0.10, 0.11, 0.12, 0.13, 0.14, 0.15, 0.16, 0.17, or 0.18 mg*L⁻¹*h⁻¹. In yet another embodiment, the production of 1-alkenes is inhibited by the presence of 15 μM urea in the culture medium.

One embodiment of the present invention also provides an isolated or recombinant polynucleotide comprising or consisting of a nucleic acid sequence selected from SEQ ID NO:6, SEQ ID NO:8, SEQ ID NO:10, SEQ ID NO:12, SEQ ID NO:14, or SEQ ID NO:16. In another embodiment, a nucleic acid sequence is provided that is a degenerate variant of SEQ ID NO:6, SEQ ID NO:8, SEQ ID NO:10, SEQ ID NO:12, SEQ ID NO:14, or SEQ ID NO:16. In still another embodiment, a nucleic acid sequence at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 90%, at least 95%, at least 98%, at least 99% or at least 99.9% identical to SEQ ID NO:6, SEQ ID NO:8, SEQ ID NO:10, SEQ ID NO:12, SEQ ID NO:14, or SEQ ID NO:16 is provided. In yet another embodiment, a nucleic acid sequence that encodes a polypeptide having the amino acid sequence of SEQ ID NO:7, SEQ ID NO:9, SEQ ID NO:11, SEQ ID NO:13, SEQ ID NO:15, or SEQ ID NO:17 is provided. Also provided by an embodiment of the invention is a nucleic acid sequence that encodes a polypeptide at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%%, at least 99.1%, at least 99.2%, at least 99.3%, at least 99.4%, at least 99.5%, at least 99.6%, at least 99.7%, at least 99.8% or at least 99.9% identical to SEQ ID NO:7, SEQ ID NO:9, SEQ ID NO:11, SEQ ID NO:13, SEQ ID NO:15, or SEQ ID NO:17. In another embodiment, a nucleic acid sequence is provided that hybridizes under stringent conditions to SEQ ID NO:6, SEQ ID NO:8, SEQ ID NO:10, SEQ ID NO:12, SEQ ID NO:14, or SEQ ID NO:16.

In one aspect, a nucleic acid sequence of the invention encodes a polypeptide having alpha-olefin synthesis associated activity. In one embodiment, the polypeptide comprises SEQ ID NO:7, SEQ ID NO:9, SEQ ID NO:11, SEQ ID NO:13, SEQ ID NO:15, or SEQ ID NO:17. In another aspect, the nucleic acid sequence and the sequence of interest are operably linked to one or more expression control sequences. In still another aspect, a vector comprising an isolated polynucleotide of the invention is provided. In one embodiment, the vector comprises a nucleotide sequence at least 90% identical to SEQ ID NO: 20. In another embodiment, the vector comprises a nucleotide sequence at least 90% identical to SEQ ID NO: 21. In still another embodiment, the vector comprises a spectinomycin resistance marker. In a further embodiment, the spectinomycin resistance marker is at least 90% identical to SEQ ID NO: 22. In yet another embodiment, the vector comprises a nucleotide sequence at least 90% identical to SEQ ID NO: 23. In yet another aspect, a polynucleotide encoding a fusion protein is provided comprising an isolated or recombinant aoa gene fused to a gene encoding a heterologous amino acid sequence.

In one embodiment, a host cell is provided comprising an isolated polynucleotide of the invention (i.e., alpha-olefin associated gene and/or 1-alkene synthase genes). In another embodiment, the host cell is selected from prokaryotes, eukaryotes, yeasts, filamentous fungi, protozoa, algae and synthetic cells. In still another embodiment, the host cell produces a carbon-based product of interest. In one aspect, the present disclosure provides an isolated antibody or antigen-binding fragment or derivative thereof which binds selectively to an isolated polypeptide of the invention.

Also provided is a method for producing carbon-based products of interest comprising culturing a recombinant host cell engineered to produce carbon-based products of interest, wherein said host cell comprises a recombinant nucleotide sequence of the invention, and removing the carbon-based product of interest. In one aspect, the recombinant nucleotide sequence encodes a polypeptide having alpha-olefin synthesis-associated activity.

In one embodiment, a method for identifying a modified gene that improves 1-alkene synthesis is provided, comprising identifying a polynucleotide sequence expressing an enzyme involved in 1-alkene biosynthesis, expressing the enzyme from a recombinant form of the polynucleotide sequence in a host cell, and screening the host cell for increased activity of said enzyme or increased production of 1-alkene.

Additional information related to the invention may be found in the following Drawings and Detailed Description.

### DRAWINGS

FIG. 1 shows a stack of GC/MS chromatograms comparing cell pellet extracts of JCC2157 and JCC308. The interval between the tick marks on the MS detector axis is 1000.

FIG. 2 shows the mass spectra of identified 1-alkenes in JCC2157 cell extracts. The MS fragmentation patterns of (A) the JCC2157 1-heptadecene peak plotted above the spectrum in the NIST database, (B) the JCC2157 1-octadecene peak plotted above the spectrum in the NIST database, and (C) the JCC2157 1-nonadecene peak plotted above the spectrum in the NIST database are shown. (D) The mass spectrum of the JCC2157 peak identified as 1,x-nonadecadiene (19:2).

FIG. 3 shows a stack of GC/FID chromatograms comparing cell pellet extracts of JCC1218, JCC138 and JCC4124. The interval between the tick marks on the FID detector axis is 2.

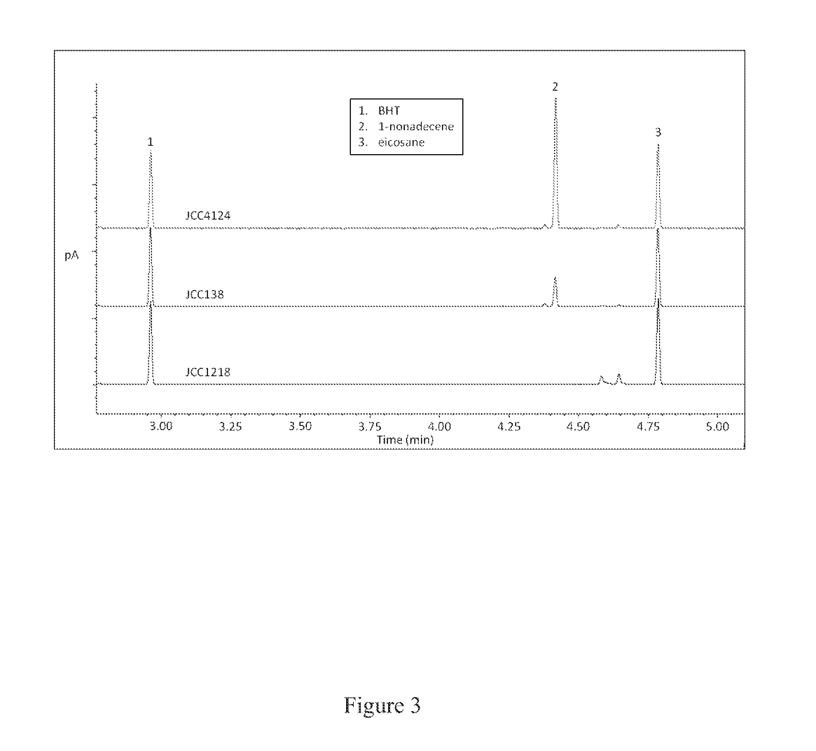

FIG. 4 shows the growth and 1-nonadecene production of the JCC1218, JCC138, and JCC4124 in 2 mM urea (U2) or 15 mM urea (U15). The plotted data is the average of the duplicate flasks and the error bars depict the high/low values of the duplicate flasks. FIG. 4A shows growth of the cultures. FIG. 4B shows 1-nonadecene production by the cultures.

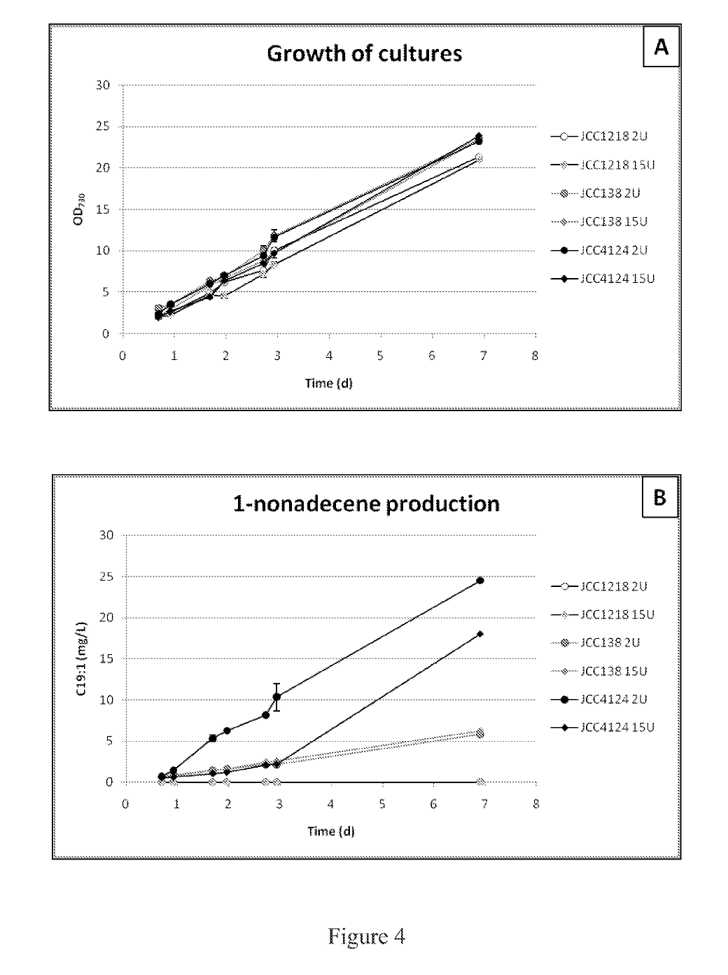

### DETAILED DESCRIPTION OF THE INVENTION

Unless otherwise defined herein, scientific and technical terms used in connection with the invention shall have the meanings that are commonly understood by those of ordinary skill in the art. Further, unless otherwise required by context, singular terms shall include the plural and plural terms shall include the singular. Generally, nomenclatures used in connection with, and techniques of, biochemistry, enzymology, molecular and cellular biology, microbiology, genetics and protein and nucleic acid chemistry and hybridization described herein are those well known and commonly used in the art. The methods and techniques are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol. I, CRC Press (1976); Handbook of Biochemistry: Section A Proteins, Vol. II, CRC Press (1976); Essentials of Glycobiology, Cold Spring Harbor Laboratory Press (1999).

The following terms, unless otherwise indicated, shall be understood to have the following meanings:

The term “polynucleotide” or “nucleic acid molecule” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native inter-nucleoside bonds, or both. The nucleic acid can be in any topological conformation. For instance, the nucleic acid can be single-stranded, double-stranded, triple-stranded, quadruplexed, partially double-stranded, branched, hair-pinned, circular, or in a padlocked conformation.

Unless otherwise indicated, and as an example for all sequences described herein under the general format “SEQ ID NO:”, “nucleic acid comprising SEQ ID NO:1” refers to a nucleic acid, at least a portion of which has either (i) the sequence of SEQ ID NO:1, or (ii) a sequence complementary to SEQ ID NO:1. The choice between the two is dictated by the context. For instance, if the nucleic acid is used as a probe, the choice between the two is dictated by the requirement that the probe be complementary to the desired target.

An “isolated” or “substantially pure” nucleic acid or polynucleotide (e.g., an RNA, DNA or a mixed polymer) is one which is substantially separated from other cellular components that naturally accompany the native polynucleotide in its natural host cell, e.g., ribosomes, polymerases and genomic sequences with which it is naturally associated. The term embraces a nucleic acid or polynucleotide that (1) has been removed from its naturally occurring environment, (2) is not associated with all or a portion of a polynucleotide in which the “isolated polynucleotide” is found in nature, (3) is operatively linked to a polynucleotide which it is not linked to in nature, or (4) does not occur in nature. The term “isolated” or “substantially pure” also can be used in reference to recombinant or cloned DNA isolates, chemically synthesized polynucleotide analogs, or polynucleotide analogs that are biologically synthesized by heterologous systems.

However, “isolated” does not necessarily require that the nucleic acid or polynucleotide so described has itself been physically removed from its native environment. For instance, an endogenous nucleic acid sequence in the genome of an organism is deemed “isolated” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “isolated” because it is separated from at least some of the sequences that naturally flank it.

A nucleic acid is also considered “isolated” if it contains any modifications that do not naturally occur to the corresponding nucleic acid in a genome. For instance, an endogenous coding sequence is considered “isolated” if it contains an insertion, deletion or a point mutation introduced artificially, e.g., by human intervention. An “isolated nucleic acid” also includes a nucleic acid integrated into a host cell chromosome at a heterologous site and a nucleic acid construct present as an episome. Moreover, an “isolated nucleic acid” can be substantially free of other cellular material or substantially free of culture medium when produced by recombinant techniques or substantially free of chemical precursors or other chemicals when chemically synthesized.

The term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids. For example, a “recombinant 1-alkene synthase” can be a protein encoded by a heterologous 1-alkene synthase gene; or a protein encoded by a duplicate copy of an endogenous 1-alkene synthase gene; or a protein encoded by a modified endogenous 1-alkene synthase gene; or a protein encoded by an endogenous 1-alkene synthase gene expressed from a heterologous promoter; or a protein encoded by an endogenous 1-alkene synthase gene where expression is driven, at least in part, by an endogenous promoter different from the organism's native 1-alkene synthase promoter.

As used herein, the phrase “degenerate variant” of a reference nucleic acid sequence encompasses nucleic acid sequences that can be translated, according to the standard genetic code, to provide an amino acid sequence identical to that translated from the reference nucleic acid sequence. The term “degenerate oligonucleotide” or “degenerate primer” is used to signify an oligonucleotide capable of hybridizing with target nucleic acid sequences that are not necessarily identical in sequence but that are homologous to one another within one or more particular segments.

The term “percent sequence identity” or “identical” in the context of nucleic acid sequences refers to the residues in the two sequences which are the same when aligned for maximum correspondence. The length of sequence identity comparison may be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA provides alignments and percent sequence identity of the regions of the best overlap between the query and search sequences. Pearson, Methods Enzymol. 183:63-98 (1990) (hereby incorporated by reference in its entirety). For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference. Alternatively, sequences can be compared using the computer program, BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

A particular, non-limiting example of a mathematical algorithm utilized for the comparison of sequences is that of Karlin and Altschul (Proc. Natl. Acad. Sci. (1990) USA 87:2264-68; Proc. Natl. Acad. Sci. USA (1993) 90: 5873-77) as used in the NBLAST and XBLAST programs (version 2.0) of Altschul et al. (J. Mol. Biol. (1990) 215:403-10). BLAST nucleotide searches can be performed with the NBLAST program, score=100, wordlength=12 to obtain nucleotide sequences homologous to nucleic acid molecules of the invention. BLAST polypeptide searches can be performed with the XBLAST program, score=50, wordlength=3 to obtain amino acid sequences homologous to polypeptide molecules of the invention. To obtain gapped alignments for comparison purposes, Gapped BLAST can be utilized as described in Altschul et al. (Nucleic Acids Research (1997) 25(17):3389-3402). When utilizing BLAST and Gapped BLAST programs, the default parameters of the respective programs (e.g., XBLAST and NBLAST) can be used (http://www.ncbi.nlm.nih.gov). One skilled in the art may also use the ALIGN program incorporating the non-linear algorithm of Myers and Miller (Comput. Appl. Biosci. (1988) 4:11-17). For amino acid sequence comparison using the ALIGN program one skilled in the art may use a PAM 120 weight residue table, a gap length penalty of 12, and a gap penalty of 4.

The term “substantial homology” or “substantial similarity,” when referring to a nucleic acid or fragment thereof, indicates that, when optimally aligned with appropriate nucleotide insertions or deletions with another nucleic acid (or its complementary strand), there is nucleotide sequence identity in at least about 71%, 72%, 73%, 74%, 75%, 76%, 77%, 78%, 79%, 80%, 81%, 82%, 83%, 84%, 85%, preferably at least about 90%, and more preferably at least about 95%, 96%, 97%, 98% or 99% of the nucleotide bases, as measured by any well-known algorithm of sequence identity, such as FASTA, BLAST or Gap, as discussed above.

Alternatively, substantial homology or similarity exists when a nucleic acid or fragment thereof hybridizes to another nucleic acid, to a strand of another nucleic acid, or to the complementary strand thereof, under stringent hybridization conditions. “Stringent hybridization conditions” and “stringent wash conditions” in the context of nucleic acid hybridization experiments depend upon a number of different physical parameters. Nucleic acid hybridization will be affected by such conditions as salt concentration, temperature, solvents, the base composition of the hybridizing species, length of the complementary regions, and the number of nucleotide base mismatches between the hybridizing nucleic acids, as will be readily appreciated by those skilled in the art. One having ordinary skill in the art knows how to vary these parameters to achieve a particular stringency of hybridization.

In general, “stringent hybridization” is performed at about 25° C. below the thermal melting point (Tₘ) for the specific DNA hybrid under a particular set of conditions. “Stringent washing” is performed at temperatures about 5° C. lower than the Tₘ for the specific DNA hybrid under a particular set of conditions. The Tₘ is the temperature at which 50% of the target sequence hybridizes to a perfectly matched probe. See Sambrook et al., Molecular Cloning: A Laboratory Manual, 2d ed., Cold Spring Harbor Laboratory Press, Cold Spring Harbor, N.Y. (1989), page 9.51, hereby incorporated by reference. For purposes herein, “stringent conditions” are defined for solution phase hybridization as aqueous hybridization (i.e., free of formamide) in 6×SSC (where 20×SSC contains 3.0 M NaCl and 0.3 M sodium citrate), 1% SDS at 65° C. for 8-12 hours, followed by two washes in 0.2×SSC, 0.1% SDS at 65° C. for 20 minutes. It will be appreciated by the skilled worker that hybridization at 65° C. will occur at different rates depending on a number of factors including the length and percent identity of the sequences which are hybridizing.

A preferred, non-limiting example of stringent hybridization conditions includes hybridization in 4× sodium chloride/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Intermediate ranges e.g., at 65-70° C. or at 42-50° C. are also within the scope of the invention. SSPE (1×SSPE is 0.15 M NaCl, 10 mM NaH₂PO₄, and 1.25 mM EDTA, pH 7.4) can be substituted for SSC (1×SSC is 0.15 M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ (° C.)=2(# of A+T bases)+4(# of G+C bases). For hybrids between 18 and 49 base pairs in length, Tₘ(° C.)=81.5+16.6(log₁₀[Na⁺])+0.41 (% G+C)−(600/N), where N is the number of bases in the hybrid, and [Na⁺] is the concentration of sodium ions in the hybridization buffer ([Na⁺] for 1×SSC=0.165 M).

The skilled practitioner recognizes that reagents can be added to hybridization and/or wash buffers. For example, to decrease non-specific hybridization of nucleic acid molecules to, for example, nitrocellulose or nylon membranes, blocking agents, including but not limited to, BSA or salmon or herring sperm carrier DNA and/or detergents, including but not limited to, SDS, chelating agents EDTA, Ficoll, PVP and the like can be used. When using nylon membranes, in particular, an additional, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C. (Church and Gilbert (1984) Proc. Natl. Acad. Sci. USA 81:1991-1995) or, alternatively, 0.2×SSC, 1% SDS.

The nucleic acids (also referred to as polynucleotides) may include both sense and antisense strands of RNA, cDNA, genomic DNA, and synthetic forms and mixed polymers of the above. They may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.) Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule. Other modifications can include, for example, analogs in which the ribose ring contains a bridging moiety or other structure such as the modifications found in “locked” nucleic acids.

The term “mutated” when applied to nucleic acid sequences means that nucleotides in a nucleic acid sequence may be inserted, deleted or changed compared to a reference nucleic acid sequence. A single alteration may be made at a locus (a point mutation) or multiple nucleotides may be inserted, deleted or changed at a single locus. In addition, one or more alterations may be made at any number of loci within a nucleic acid sequence. A nucleic acid sequence may be mutated by any method known in the art including but not limited to mutagenesis techniques such as “error-prone PCR” (a process for performing PCR under conditions where the copying fidelity of the DNA polymerase is low, such that a high rate of point mutations is obtained along the entire length of the PCR product; see, e.g., Leung et al., Technique, 1:11-15 (1989) and Caldwell and Joyce, PCR Methods Applic. 2:28-33 (1992)); and “oligonucleotide-directed mutagenesis” (a process which enables the generation of site-specific mutations in any cloned DNA segment of interest; see, e.g., Reidhaar-Olson and Sauer, Science 241:53-57 (1988)).

The term “derived from” is intended to include the isolation (in whole or in part) of a polynucleotide segment from an indicated source. The term is intended to include, for example, direct cloning, PCR amplification, or artificial synthesis from, or based on, a sequence associated with the indicated polynucleotide source.

The term “gene” as used herein refers to a nucleotide sequence that can direct synthesis of an enzyme or other polypeptide molecule (e.g., can comprise coding sequences, for example, a contiguous open reading frame (ORF) which encodes a polypeptide) or can itself be functional in the organism. A gene in an organism can be clustered within an operon, as defined herein, wherein the operon is separated from other genes and/or operons by intergenic DNA. Individual genes contained within an operon can overlap without intergenic DNA between the individual genes.

An “isolated gene,” as described herein, includes a gene which is essentially free of sequences which naturally flank the gene in the chromosomal DNA of the organism from which the gene is derived (i.e., is free of adjacent coding sequences which encode a second or distinct polypeptide or RNA molecule, adjacent structural sequences or the like) and optionally includes 5′ and 3′ regulatory sequences, for example promoter sequences and/or terminator sequences. In one embodiment, an isolated gene includes predominantly coding sequences for a polypeptide.

The term “expression” when used in relation to the transcription and/or translation of a nucleotide sequence as used herein generally includes expression levels of the nucleotide sequence being enhanced, increased, resulting in basal or housekeeping levels in the host cell, constitutive, attenuated, decreased or repressed.

The term “attenuate” as used herein generally refers to a functional deletion, including a mutation, partial or complete deletion, insertion, or other variation made to a gene sequence or a sequence controlling the transcription of a gene sequence, which reduces or inhibits production of the gene product, or renders the gene product non-functional. In some instances a functional deletion is described as a knockout mutation. Attenuation also includes amino acid sequence changes by altering the nucleic acid sequence, placing the gene under the control of a less active promoter, down-regulation, expressing interfering RNA, ribozymes or antisense sequences that target the gene of interest, or through any other technique known in the art. In one example, the sensitivity of a particular enzyme to feedback inhibition or inhibition caused by a composition that is not a product or a reactant (non-pathway specific feedback) is lessened such that the enzyme activity is not impacted by the presence of a compound. In other instances, an enzyme that has been altered to be less active can be referred to as attenuated.

A “deletion” is the removal of one or more nucleotides from a nucleic acid molecule or one or more amino acids from a protein, the regions on either side being joined together.

A “knock-out” is a gene whose level of expression or activity has been reduced to zero. In some examples, a gene is knocked-out via deletion of some or all of its coding sequence. In other examples, a gene is knocked-out via introduction of one or more nucleotides into its open-reading frame, which results in translation of a non-sense or otherwise non-functional protein product.

The term “codon usage” is intended to refer to analyzing a nucleic acid sequence to be expressed in a recipient host organism (or acellular extract thereof) for the occurrence and use of preferred codons the host organism transcribes advantageously for optimal nucleic acid sequence transcription. The recipient host may be recombinantly altered with any preferred codon. Alternatively, a particular cell host can be selected that already has superior codon usage, or the nucleic acid sequence can be genetically engineered to change a limiting codon to a non-limiting codon (e.g., by introducing a silent mutation(s)).

The term “vector” as used herein is intended to refer to a nucleic acid molecule capable of transporting another nucleic acid to which it has been linked. One type of vector is a “plasmid,” which refers to a circular double stranded DNA loop into which additional DNA segments may be ligated. Other vectors include cosmids, bacterial artificial chromosomes (BAC) and yeast artificial chromosomes (YAC), fosmids, phage and phagemids. Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain preferred vectors are capable of directing the expression of genes to which they are operatively linked. Such vectors are referred to herein as “recombinant expression vectors” (or simply “expression vectors”).

“Expression optimization” as used herein is defined as one or more optional modifications to the nucleotide sequence in the promoter and terminator elements resulting in desired rates and levels of transcription and translation into a protein product encoded by said nucleotide sequence. Expression optimization as used herein also includes designing an effectual predicted secondary structure (for example, stem-loop structures and termination sequences) of the messenger ribonucleic acid (mRNA) sequence to promote desired levels of protein production. Other genes and gene combinations essential for the production of a protein may be used, for example genes for proteins in a biosynthetic pathway, required for post-translational modifications or required for a heteromultimeric protein, wherein combinations of genes are chosen for the effect of optimizing expression of the desired levels of protein product. Conversely, one or more genes optionally may be “knocked-out” or otherwise altered such that lower or eliminated expression of said gene or genes achieves the desired expression levels of protein. Additionally, expression optimization can be achieved through codon optimization. Codon optimization, as used herein, is defined as modifying a nucleotide sequence for effectual use of host cell bias in relative concentrations of transfer ribonucleic acids (tRNA) such that the desired rate and levels of gene nucleotide sequence translation into a final protein product are achieved, without altering the peptide sequence encoded by the nucleotide sequence.

The term “expression control sequence” as used herein refers to polynucleotide sequences which are necessary to affect the expression of coding sequences to which they are operatively linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

“Operatively linked” or “operably linked” expression control sequences refers to a linkage in which the expression control sequence is contiguous with the gene of interest to control the gene of interest, as well as expression control sequences that act in trans or at a distance to control the gene of interest.

The term “recombinant host cell” (or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism.

The term “peptide” as used herein refers to a short polypeptide, e.g., one that is typically less than about 50 amino acids long and more typically less than about 30 amino acids long. The term as used herein encompasses analogs and mimetics that mimic structural and thus biological function.

The term “polypeptide” encompasses both naturally-occurring and non-naturally-occurring proteins, and fragments, mutants, derivatives and analogs thereof. A polypeptide may be monomeric or polymeric. Further, a polypeptide may comprise a number of different domains each of which has one or more distinct activities.

The term “isolated protein” or “isolated polypeptide” is a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art. As thus defined, “isolated” does not necessarily require that the protein, polypeptide, peptide or oligopeptide so described has been physically removed from its native environment.

An isolated or purified polypeptide is substantially free of cellular material or other contaminating polypeptides from the expression host cell from which the polypeptide is derived, or substantially free from chemical precursors or other chemicals when chemically synthesized. In one embodiment, an isolated or purified polypeptide has less than about 30% (by dry weight) of contaminating polypeptide or chemicals, more advantageously less than about 20% of contaminating polypeptide or chemicals, still more advantageously less than about 10% of contaminating polypeptide or chemicals, and most advantageously less than about 5% contaminating polypeptide or chemicals.

The term “polypeptide fragment” as used herein refers to a polypeptide that has a deletion, e.g., an amino-terminal and/or carboxy-terminal deletion compared to a full-length polypeptide. In a preferred embodiment, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, preferably at least 12, 14, 16 or 18 amino acids long, more preferably at least 20 amino acids long, more preferably at least 25, 30, 35, 40 or 45, amino acids, even more preferably at least 50 or 60 amino acids long, and even more preferably at least 70 amino acids long.

A “modified derivative” refers to polypeptides or fragments thereof that are substantially homologous in primary structural sequence but which include, e.g., in vivo or in vitro chemical and biochemical modifications or which incorporate amino acids that are not found in the native polypeptide. Such modifications include, for example, acetylation, carboxylation, phosphorylation, glycosylation, ubiquitination, labeling, e.g., with radionuclides, and various enzymatic modifications, as will be readily appreciated by those skilled in the art. A variety of methods for labeling polypeptides and of substituents or labels useful for such purposes are well known in the art, and include radioactive isotopes such as ¹²⁵I, ³²P, ³⁵S, and ³H, ligands which bind to labeled antiligands (e.g., antibodies), fluorophores, chemiluminescent agents, enzymes, and antiligands which can serve as specific binding pair members for a labeled ligand. The choice of label depends on the sensitivity required, ease of conjugation with the primer, stability requirements, and available instrumentation. Methods for labeling polypeptides are well known in the art. See, e.g., Ausubel et al., Current Protocols in Molecular Biology, Greene Publishing Associates (1992, and Supplements to 2002) (hereby incorporated by reference).

The terms “thermal stability” and “thermostability” are used interchangeably and refer to the ability of an enzyme (e.g., whether expressed in a cell, present in an cellular extract, cell lysate, or in purified or partially purified form) to exhibit the ability to catalyze a reaction at least at about 20° C., preferably at about 25° C. to 35° C., more preferably at about 37° C. or higher, in more preferably at about 50° C. or higher, and even more preferably at least about 60° C. or higher.

The term “chimeric” refers to an expressed or translated polypeptide in which a domain or subunit of a particular homologous or non-homologous protein is genetically engineered to be transcribed, translated and/or expressed collinearly in the nucleotide and amino acid sequence of another homologous or non-homologous protein.

The term “fusion protein” refers to a polypeptide comprising a polypeptide or fragment coupled to heterologous amino acid sequences. Fusion proteins are useful because they can be constructed to contain two or more desired functional elements from two or more different proteins. A fusion protein comprises at least 10 contiguous amino acids from a polypeptide of interest, more preferably at least 20 or 30 amino acids, even more preferably at least 40, 50 or 60 amino acids, yet more preferably at least 75, 100 or 125 amino acids. Fusions that include the entirety of the proteins have particular utility. The heterologous polypeptide included within the fusion protein is at least 6 amino acids in length, often at least 8 amino acids in length, and usefully at least 15, 20, and 25 amino acids in length. Fusions that include larger polypeptides, such as an IgG Fc region, and even entire proteins, such as the green fluorescent protein (“GFP”) chromophore-containing proteins, have particular utility. Fusion proteins can be produced recombinantly by constructing a nucleic acid sequence which encodes the polypeptide or a fragment thereof in frame with a nucleic acid sequence encoding a different protein or peptide and then expressing the fusion protein. Alternatively, a fusion protein can be produced chemically by crosslinking the polypeptide or a fragment thereof to another protein.

As used herein, the term “protomer” refers to a polymeric form of amino acids forming a subunit of a larger oligomeric protein structure. Protomers of an oligomeric structure may be identical or non-identical. Protomers can combine to form an oligomeric subunit, which can combine further with other identical or non-identical protomers to form a larger oligomeric protein.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives.

Fragments within the scope of the term “antibody” include those produced by digestion with various proteases, those produced by chemical cleavage and/or chemical dissociation and those produced recombinantly, so long as the fragment remains capable of specific binding to a target molecule. Among such fragments are Fab, Fab′, Fv, F(ab′)₂, and single chain Fv (scFv) fragments.

Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Intracellular Antibodies: Research and Disease Applications (1998) Marasco, ed., Springer-Verlag New York, Inc.), the disclosure of which is incorporated herein by reference in its entirety).

As used herein, antibodies can be produced by any known technique, including harvest from cell culture of native B lymphocytes, harvest from culture of hybridomas, recombinant expression systems and phage display.

The term “non-peptide analog” refers to a compound with properties that are analogous to those of a reference polypeptide. A non-peptide compound may also be termed a “peptide mimetic” or a “peptidomimetic.” See, e.g., Jones, Amino Acid and Peptide Synthesis, Oxford University Press (1992); Jung, Combinatorial Peptide and Nonpeptide Libraries: A Handbook, John Wiley (1997); Bodanszky et al., Peptide Chemistry—A Practical Textbook, Springer Verlag (1993); Synthetic Peptides: A Users Guide, (Grant, ed., W.H. Freeman and Co., 1992); Evans et al., J. Med. Chem. 30:1229 (1987); Fauchere, J. Adv. Drug Res. 15:29 (1986); Veber and Freidinger, Trends Neurosci., 8:392-396 (1985); and references sited in each of the above, which are incorporated herein by reference. Such compounds are often developed with the aid of computerized molecular modeling. Peptide mimetics that are structurally similar to useful peptides may be used to produce an equivalent effect and are therefore envisioned to be part of the invention.

A “polypeptide mutant” or “mutein” refers to a polypeptide whose sequence contains an insertion, duplication, deletion, rearrangement or substitution of one or more amino acids compared to the amino acid sequence of a native or wild-type protein. A mutein may have one or more amino acid point substitutions, in which a single amino acid at a position has been changed to another amino acid, one or more insertions and/or deletions, in which one or more amino acids are inserted or deleted, respectively, in the sequence of the naturally-occurring protein, and/or truncations of the amino acid sequence at either or both the amino or carboxy termini. A mutein may have the same but preferably has a different biological activity compared to the naturally-occurring protein.

A mutein has at least 85% overall sequence homology to its wild-type counterpart. Even more preferred are muteins having at least 90% overall sequence homology to the wild-type protein.

In an even more preferred embodiment, a mutein exhibits at least 95% sequence identity, even more preferably 98%, even more preferably 99% and even more preferably 99.9% overall sequence identity.

Sequence homology may be measured by any common sequence analysis algorithm, such as Gap or Bestfit.

Amino acid substitutions can include those which: (1) reduce susceptibility to proteolysis, (2) reduce susceptibility to oxidation, (3) alter binding affinity for forming protein complexes, (4) alter binding affinity or enzymatic activity, and (5) confer or modify other physicochemical or functional properties of such analogs.

As used herein, the twenty conventional amino acids and their abbreviations follow conventional usage. See Immunology—A Synthesis (Golub and Gren eds., Sinauer Associates, Sunderland, Mass., 2^{nd} ed. 1991), which is incorporated herein by reference. Stereoisomers (e.g., D-amino acids) of the twenty conventional amino acids, unnatural amino acids such as α-, α-disubstituted amino acids, N-alkyl amino acids, and other unconventional amino acids may also be suitable components for polypeptides. Examples of unconventional amino acids include: 4-hydroxyproline, γ-carboxyglutamate, ε-N,N,N-trimethyllysine, ε-N-acetyllysine, 0-phosphoserine, N-acetylserine, N-formylmethionine, 3-methylhistidine, 5-hydroxylysine, N-methylarginine, and other similar amino acids and imino acids (e.g., 4-hydroxyproline). In the polypeptide notation used herein, the left-hand end corresponds to the amino terminal end and the right-hand end corresponds to the carboxy-terminal end, in accordance with standard usage and convention.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences.) As used herein, homology between two regions of amino acid sequence (especially with respect to predicted structural similarities) is interpreted as implying similarity in function.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-331 and 25:365-389 (herein incorporated by reference).

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using a measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutein thereof. See, e.g., GCG Version 6.1.

A preferred algorithm when comparing a particular polypeptide sequence to a database containing a large number of sequences from different organisms is the computer program BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences. (Pearson, Methods Enzymol. 183:63-98 (1990) (herein incorporated by reference). For example, percent sequence identity between amino acid sequences can be determined using FASTA with its default parameters (a word size of 2 and the PAM250 scoring matrix), as provided in GCG Version 6.1, herein incorporated by reference.

To determine the percent identity of two amino acid sequences or of two nucleic acids, the sequences are aligned for optimal comparison purposes, and, if necessary, gaps can be introduced in the first amino acid or nucleic acid sequence for optimal alignment with a second amino or nucleic acid sequence. When a position in the first sequence is occupied by the same amino acid residue or nucleotide as the corresponding position in the second sequence, then the molecules are identical at that position. The percent identity between the two sequences is a function of the number of identical positions shared by the sequences as evaluated, for example, by calculating # of identical positions/total # of positions×100. Additional evaluations of the sequence alignment can include a numeric penalty taking into account the number of gaps and size of said gaps necessary to produce an optimal alignment.

“Specific binding” refers to the ability of two molecules to bind to each other in preference to binding to other molecules in the environment. Typically, “specific binding” discriminates over adventitious binding in a reaction by at least two-fold, more typically by at least 10-fold, often at least 100-fold. Typically, the affinity or avidity of a specific binding reaction, as quantified by a dissociation constant, is about 10⁻⁷ M or stronger (e.g., about 10⁻⁸ M, 10⁻⁹ M or even stronger).

The term “region” as used herein refers to a physically contiguous portion of the primary structure of a biomolecule. In the case of proteins, a region is defined by a contiguous portion of the amino acid sequence of that protein.

The term “domain” as used herein refers to a structure of a biomolecule that contributes to a known or suspected function of the biomolecule. Domains may be co-extensive with regions or portions thereof; domains may also include distinct, non-contiguous regions of a biomolecule. Examples of protein domains include, but are not limited to, an Ig domain, an extracellular domain, a transmembrane domain, and a cytoplasmic domain.

As used herein, the term “molecule” means any compound, including, but not limited to, a small molecule, peptide, protein, sugar, nucleotide, nucleic acid, lipid, etc., and such a compound can be natural or synthetic.

The term “substrate affinity” as used herein refers to the binding kinetics, Kₘ, the Michaelis-Menten constant as understood by one having skill in the art, for a substrate. More particularly the Kₘ is optimized over endogenous activity for the purpose of the invention described herein.

The term “sugar” as used herein refers to any carbohydrate endogenously produced from sunlight, a carbon source, and water, any carbohydrate produced endogenously and/or any carbohydrate from any exogenous carbon source such as biomass, comprising a sugar molecule or pool or source of such sugar molecules.

The term “carbon source” as used herein refers to carbon dioxide, exogenous sugar or biomass, or another inorganic carbon source.

“Carbon-based products of interest” include alcohols such as ethanol, propanol, isopropanol, butanol, fatty alcohols, fatty acid esters, wax esters; hydrocarbons and alkanes such as propane, octane, diesel, Jet Propellant 8 (JP8); polymers such as 1-nonadecene, terephthalate, 1,3-propanediol, 1,4-butanediol, polyols, Polyhydroxyalkanoates (PHA), poly-beta-hydroxybutyrate (PHB), acrylate, adipic acid, ε-caprolactone, isoprene, caprolactam, rubber; commodity chemicals such as lactate, docosahexaenoic acid (DHA), 3-hydroxypropionate, γ-valerolactone, lysine, serine, aspartate, aspartic acid, sorbitol, ascorbate, ascorbic acid, isopentenol, lanosterol, omega-3 DHA, lycopene, itaconate, 1,3-butadiene, ethylene, propylene, succinate, citrate, citric acid, glutamate, malate, 3-hydroxypropionic acid (HPA), lactic acid, THF, gamma butyrolactone, pyrrolidones, hydroxybutyrate, glutamic acid, levulinic acid, acrylic acid, malonic acid; specialty chemicals such as carotenoids, isoprenoids, itaconic acid; pharmaceuticals and pharmaceutical intermediates such as 7-aminodeacetoxycephalosporanic acid (7-ADCA)/cephalosporin, erythromycin, polyketides, statins, paclitaxel, docetaxel, terpenes, peptides, steroids, omega fatty acids, olefins, alkenes and other such suitable products of interest. Such products are useful in the context of biofuels, industrial and specialty chemicals, as intermediates used to make additional products, such as nutritional supplements, neutraceuticals, polymers, paraffin replacements, personal care products and pharmaceuticals.

A “biofuel” as used herein is any fuel that derives from a biological source. A “fuel” refers to one or more hydrocarbons (e.g., 1-alkenes), one or more alcohols, one or more fatty esters or a mixture thereof. Preferably, liquid hydrocarbons are used.

As used herein, the term “hydrocarbon” generally refers to a chemical compound that consists of the elements carbon (C), hydrogen (H) and optionally oxygen (O). There are essentially three types of hydrocarbons, e.g., aromatic hydrocarbons, saturated hydrocarbons and unsaturated hydrocarbons such as alkenes, alkynes, and dienes. The term also includes fuels, biofuels, plastics, waxes, solvents and oils. Hydrocarbons encompass biofuels, as well as plastics, waxes, solvents and oils.

Polyketide synthases are enzymes or enzyme complexes that produce polyketides, a large class of secondary metabolites in bacteria, fungi, plants and animals. The invention described herein provides a recombinant 1-alkene synthase gene, which is related to type I polyketides synthases. As used herein, a “1-alkene synthase” is an enzyme whose BLAST alignment covers 90% of the length of SEQ ID NO:3 or SEQ ID NO:5 and has at least 50% identity to the amino acid sequence of SEQ ID NO:3 or SEQ ID NO:5, and (2) which catalyzes the synthesis of 1-alkenes. A 1-alkene synthase is referred to herein as NonA; the corresponding gene may be referred to as nonA. An improved 1-alkene synthase enzyme is also provided in SEQ ID NO:3 (nonA_optV6). In one embodiment, an improved 1-alkene synthase enzyme is also provided, whose BLAST alignment covers 90% of the length of SEQ ID NO:3 (nonA_optV6) and has at least 50% identity to the amino acid sequence of SEQ ID NO:3.

An exemplary 1-alkene synthase is the 1-alkene synthase of Synechococcus sp. PCC 7002 (SEQ ID NO: 5). An exemplary gene encoding a 1-alkene synthase is the nonA gene of Synechococcus sp. PCC 7002 (SEQ ID NO:4). Other exemplary 1-alkene synthases are YP_{—}002377174.1 from Cyanothece sp. PCC7424 and ZP_{—}03153601.1 from Cyanothece sp. PCC7822. The amino acid sequences of these genes as they appear in the NCBI database on Aug. 17, 2011 are hereby incorporated by reference. The invention also provides 1-alkene synthases that are at least 95% identical to SEQ ID NO:2, or at least 95% identical to YP_{—}002377174.1 or at least 95% identical to ZP_{—}03153601.1, in addition to engineered microorganisms expressing genes encoding these 1-alkene synthases and methods of producing 1-alkenes by culturing these microorganisms.

The invention also provides an isolated or recombinant broad spectrum phosphopantetheinyl transferase, which refers to a gene encoding a transferase with an amino acid sequence that is at least 95% identical to the enzyme encoded by the sfp gene from Bacillus subtilis or at least 95% identical to the enzyme encoded by SEQ ID NO: 1 (Genbank ID: P39135.2).

The invention also provides an isolated or recombinant alpha-olefin-associated (Aoa) enzymes and aoa genes encoding the Aoa enzymes. This class of genes is involved in the production of 1-alkenes. In one embodiment, the invention provides the combination of the expression of aoa genes with genes encoding 1-alkene synthases in a microorganism as described above. This combination increases the production of 1-alkenes in cultured microorganisms.

As used herein, an “alpha-olefin-associated enzyme” is an enzyme which is encoded by a gene in the alpha-olefin-associated (aoa) locus of a microorganism. In one particular example, the Aoa enzyme (1) comprises regions homologous or identical to each of the domains identified in Table 1, or whose BLAST alignment covers 90% of the length of an amino acid provided in Table 1 and has at least 50% identity to the same amino acid, i.e., an alpha-olefin-associated enzyme identified in Table 1, which increases the synthesis of 1-alkenes. The alpha-olefin-associated enzyme is also referred to herein as Aoa; the corresponding gene may be referred to as aoa.

TABLE 1
1-alkene synthase (nonA) and aoa loci and NCBI protein sequence numbers

| Bacterium | 1-alkene gene locus | aoa locus | Aoa Genbank # |
|---|---|---|---|
| Synechococcus sp. PCC 7002 | SYNPCC7002_A1173 | SYNPCC7002_A2265 | YP_001735499 |
| Cyanothece sp. PCC 7822 | Cyan7822_1847 | Cyan7822_1848 | YP_003887108.1 |
| Cyanothece sp. PCC 7424 | PCC7424_1874 | PCC7424_1875 | YP_002377175 |
| Lyngbya majuscula 3L | LYNGBM3L_11280¹ | LYNGBM3L_11290 | ZP_08425909.1 |
| Lyngbya majuscula 3L | LYNGBM3L_74580² | LYNGBM3L_74520 | ZP_08432358 |
| Haliangium ochraceum | Hoch_0799³ | Hoch_0800 | YP_003265309 |
| DSM 14365 | | | |

¹This gene has a similar domain architecture to NonA and is adjacent to LYNGBM3L_11290 on the genome. It is currently unknown if the strain makes a linear fatty-acid-derived α-olefin.
²This is curM which has been implicated in terminal alkene biosynthesis (Gu et al. 2009) and is located adjacent on the genome to LYNGBM3L_74520.
³Hoch_0799 is located immediately upstream of Hoch_0800 and is a polyketide synthase gene bearing the sulfotransferase-thioesterase domain set implicated in terminal alkene formation (Gu et al. 2009).

An exemplary alpha-olefin-associated enzyme is the alpha-olefin-associated enzyme of Synechococcus sp. PCC 7002 (SEQ ID NO: 7). An exemplary gene encoding an alpha-olefin-associated enzyme is the aoa gene of Synechococcus sp. PCC 7002 (SEQ ID NO:6). Another exemplary alpha-olefin-associated enzyme is encoded by a gene whose BLAST alignment covers at least 90% of the length of SEQ ID NO:6 and has at least 50% identity with SEQ ID NO:6. Another exemplary alpha-olefin-associated enzyme is YP_{—}003887108.1 from Cyanothece sp. PCC 7822 (SEQ ID NO: 9), or an alpha-olefin-associated enzyme encoded by a gene whose BLAST alignment covers at least 90% of the length of SEQ ID NO:8 and has at least 50% identity with SEQ ID NO:8. Still another exemplary alpha-olefin-associated enzyme is YP_{—}002377175 from Cyanothece sp. PCC 7424 (SEQ ID NO:11), or an alpha-olefin-associated enzyme encoded by a gene whose BLAST alignment covers at least 90% of the length of SEQ ID NO:10 and has at least 50% identity with SEQ ID NO:10. Yet another exemplary alpha-olefin-associated enzyme is ZP_{—}08425909.1 from Lyngbya majuscule 3L (SEQ ID NO: 13), or an alpha-olefin-associated enzyme encoded by a gene whose BLAST alignment covers at least 90% of the length of SEQ ID NO:12 and has at least 50% identity with SEQ ID NO:12. A further exemplary alpha-olefin-associated enzyme is ZP_{—}08432358 from Lyngbya majuscule 3L (SEQ ID NO: 15), or an alpha-olefin-associated enzyme encoded by a gene whose BLAST alignment covers at least 90% of the length of SEQ ID NO:14 and has at least 50% identity with SEQ ID NO:14. Still another exemplary alpha-olefin-associated enzyme is YP_{—}003265309 from Haliangium ochraceum DSM 14365 (SEQ ID NO: 17), or an alpha-olefin-associated enzyme encoded by a gene whose BLAST alignment covers at least 90% of the length of SEQ ID NO:16 and has at least 50% identity with SEQ ID NO:16. The amino acid sequences of these genes as they appear in the NCBI database on Aug. 17, 2011 are hereby incorporated by reference.

The invention also provides alpha-olefin-associated enzymes that are at least 95% identical to SEQ ID NO:7, or at least 95% identical to SEQ ID NO:9, or at least 95% identical to SEQ ID NO:11, or at least 95% identical to SEQ ID NO:13, or at least 95% identical to SEQ ID NO:15, or at least 95% identical to SEQ ID NO:17, in addition to engineered microorganisms expressing genes encoding these alpha-olefin-associated enzymes and methods of producing 1-alkenes by culturing these microorganisms. Engineered microorganisms are also provided expressing genes encoding these alpha-olefin-associated enzymes and encoding 1-alkene synthases and methods of producing 1-alkenes by culturing these microorganisms.

The Billing Module 404 is configured for processing the billing to the learning user 102 for the purchase of a microlearning application 300, as well as other purchase items like access to tutoring user 112 for 1 hour during the performance of microlearning application 300, access to learning facility 132 for two hours for performance of learning application 300, purchase of a compatible learning material or tools for the performance of learning application 300, purchase of a learning workshop involving the performance of learning application 300 five times for practice, and other purchase items.

Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

The term “catabolic” and “catabolism” as used herein refers to the process of molecule breakdown or degradation of large molecules into smaller molecules. Catabolic or catabolism refers to a specific reaction pathway wherein the molecule breakdown occurs through a single or multitude of catalytic components or a general, whole cell process wherein the molecule breakdown occurs using more than one specified reaction pathway and a multitude of catalytic components.

The term “anabolic” and “anabolism” as used herein refers to the process of chemical construction of small molecules into larger molecules. Anabolic refers to a specific reaction pathway wherein the molecule construction occurs through a single or multitude of catalytic components or a general, whole cell process wherein the molecule construction occurs using more than one specified reaction pathway and a multitude of catalytic components.

The term “correlated” in “correlated saturation mutagenesis” as used herein refers to altering an amino acid type at two or more positions of a polypeptide to achieve an altered functional or structural attribute differing from the structural or functional attribute of the polypeptide from which the changes were made.

Unless otherwise defined, all technical and scientific terms used herein have the same meaning as commonly understood by one of ordinary skill in the art to which this invention pertains. Exemplary methods and materials are described below, although methods and materials similar or equivalent to those described herein can also be used and will be apparent to those of skill in the art. All publications and other references mentioned herein are incorporated by reference in their entirety. In case of conflict, the present specification, including definitions, will control. The materials, methods, and examples are illustrative only and not intended to be limiting.

Throughout this specification and claims, the word “comprise” or variations such as “comprises” or “comprising”, will be understood to imply the inclusion of a stated integer or group of integers but not the exclusion of any other integer or group of integers.

### Nucleic Acid Sequences

The cyanobacterium Synechococcus sp. PCC7002 (formerly, Agmenellum quadruplicatum) has been shown to produce the linear alpha olefin 1-nonadecene (Winters et al. 1969). Strains which produce this metabolite also produce a nonadecadiene as a minor metabolite (Winters et al. 1969) which has been identified as 1,14-(cis)-nonadecadiene (Goodloe and Light, 1982). Feeding of ¹⁴C-labelled stearic acid resulted in incorporation of the fatty acid into 1-nonadecene demonstrating that the olefin is derived from fatty acid biosynthesis (Goodloe and Light, 1982) but the enzyme or enzymes responsible for the production of the olefin was not identified.

An object of the invention described herein is to recombinantly express in a host cell genes encoding 1-alkene synthase and alpha-olefin-associated enzyme to produce 1-alkenes, including 1-nonadecene and 1-octadecene, and other carbon-based products of interest. The genes can be over-expressed in a Synechococcus strain such as JCC138 (Synechococcus sp. PCC 7002) or any other photosynthetic organism to produce a hydrocarbon from light and an inorganic carbon source (e.g., carbon dioxide). They can also be expressed in non-photosynthetic organisms to produce hydrocarbons from sugar sources. Accordingly, the invention provides isolated nucleic acid molecules encoding enzymes having 1-alkene synthase and alpha-olefin-associated enzyme activity, and variants thereof, including expression optimized forms of said genes, and methods of improvement thereon. The full-length nucleic acid sequence (SEQ ID NO:6) for the alpha-olefin-associated enzyme gene from Synechococcus sp. PCC 7002YP_{—}001735499, is provided herein, as is the protein sequence (SEQ ID NO:7).

Also provided herein is a coding (SEQ ID NO:2) and amino acid sequence (SEQ ID NO:3) for modified 1-alkene synthase, as defined above. An exemplary 1-alkene synthase is the synthase from Synechococcus sp. PCC 7002. In Synechococcus sp. PCC7002, this gene is not close to aoa on the chromosome. In the other three cyanobacteria bearing aoa homologs, the 1-alkene synthases are located immediately upstream of the aoa homolog in an apparent operon (see Table 1 for gene loci and NCBI Genbank protein reference sequence numbers).

In one embodiment is provided an isolated nucleic acid molecule having a nucleic acid sequence comprising or consisting of alpha-olefin-associated gene homologs, variants and derivatives of the wild-type alpha-olefin-associated gene coding sequence SEQ ID NO:6. The invention provides nucleic acid molecules comprising or consisting of sequences which are structurally and functionally optimized versions of the wild-type or native alpha-olefin-associated gene. In a preferred embodiment, nucleic acid molecules and homologs, variants and derivatives comprising or consisting of sequences optimized for substrate affinity and/or substrate catalytic conversion rate are provided.

In other embodiments, the invention provides vectors constructed for the preparation of aoa and nonA_optV6 strains of Synechococcus sp. PCC7002 and other cyanobacterial strains. These vectors contain sufficient lengths of upstream and downstream sequences relative to the respective gene flanking a selectable marker, e.g., an antibiotic resistance marker (gentamycin, kanamycin, ampicillin, etc.), such that recombination with the vector replaces the chromosomal copy of the gene with the antibiotic resistance gene. Exemplary examples of such vectors are provided herein.

In a further embodiment is provided nucleic acid molecules and homologs, variants and derivatives thereof comprising or consisting of sequences which are variants of the aoa gene having at least 71% identity to SEQ ID NO:6. In a further embodiment provided nucleic acid molecules and homologs, variants and derivatives comprising or consisting of sequences which are variants of the aoa gene having at least 50% identity to SEQ ID NO:6 and optimized for substrate affinity, substrate catalytic conversion rate, improved thermostability, activity at a different pH and/or optimized codon usage for improved expression in a host cell. The nucleic acid sequences can be preferably 71%, 72%, 73%, 74%, 75%, 76%, 77%, 78%, 79%, 80%, 81%, 82%, 83%, 84%, 85%, 90%, 95%, 98%, 99%, 99.9% or even higher identity to the wild-type gene.

In a further embodiment is provided nucleic acid molecules and homologs, variants and derivatives thereof comprising or consisting of sequences which are variants of the 1-alkene synthase gene having at least 71% identity to SEQ ID NO:2. In a further embodiment provided nucleic acid molecules and homologs, variants and derivatives comprising or consisting of sequences which are variants of the 1-alkene synthase gene having at least 50% identity to SEQ ID NO:2 and optimized for substrate affinity, substrate catalytic conversion rate, improved thermostability, activity at a different pH and/or optimized codon usage for improved expression in a host cell. The nucleic acid sequences can be preferably 71%, 72%, 73%, 74%, 75%, 76%, 77%, 78%, 79%, 80%, 81%, 82%, 83%, 84%, 85%, 90%, 95%, 98%, 99%, 99.9% or even higher identity to the recombinant gene (SEQ ID NO:2).

In a further embodiment is provided nucleic acid molecules and homologs, variants and derivatives thereof comprising or consisting of sequences which are variants of the phosphopantetheinyl transferase gene having at least 71% identity to SEQ ID NO:1. In a further embodiment provided nucleic acid molecules and homologs, variants and derivatives comprising or consisting of sequences which are variants of the phosphopantetheinyl transferase gene having at least 50% identity to SEQ ID NO:1 and optimized for substrate affinity, substrate catalytic conversion rate, improved thermostability, activity at a different pH and/or optimized codon usage for improved expression in a host cell. The nucleic acid sequences can be preferably 71%, 72%, 73%, 74%, 75%, 76%, 77%, 78%, 79%, 80%, 81%, 82%, 83%, 84%, 85%, 90%, 95%, 98%, 99%, 99.9% or even higher identity to the codon-optimized phosphopantetheinyl transferase gene (SEQ ID NO:1).

In another embodiment, the nucleic acid molecule encodes a polypeptide having the amino acid sequence of SEQ ID NO:1, SEQ ID NO:2 and/or SEQ NO:6. Also provided is a nucleic acid molecule encoding a polypeptide sequence that is at least 50% identical to either SEQ ID NO:1, SEQ ID NO:2, or SEQ ID NO:6. Preferably, the nucleic acid molecule encodes a polypeptide sequence of at least 55%, 60%, 70%, 80%, 90% or 95% identical to SEQ ID NO:1, SEQ ID NO:2, or SEQ ID NO:6, and the identity can even more preferably be 98%, 99%, 99.9% or even higher.

Provided also are nucleic acid molecules that hybridize under stringent conditions to the above-described nucleic acid molecules. As defined above, and as is well known in the art, stringent hybridizations are performed at about 25° C. below the thermal melting point (Tₘ) for the specific DNA hybrid under a particular set of conditions, where the Tₘ is the temperature at which 50% of the target sequence hybridizes to a perfectly matched probe. Stringent washing can be performed at temperatures about 5° C. lower than the Tₘ for the specific DNA hybrid under a particular set of conditions.

The nucleic acid molecule includes DNA molecules (e.g., linear, circular, cDNA, chromosomal DNA, double stranded or single stranded) and RNA molecules (e.g., tRNA, rRNA, mRNA) and analogs of the DNA or RNA molecules of the described herein using nucleotide analogs. The isolated nucleic acid molecule of the invention includes a nucleic acid molecule free of naturally flanking sequences (i.e., sequences located at the 5′ and 3′ ends of the nucleic acid molecule) in the chromosomal DNA of the organism from which the nucleic acid is derived. In various embodiments, an isolated nucleic acid molecule can contain less than about 10 kb, 5 kb, 4 kb, 3 kb, 2 kb, 1 kb, 0.5 kb, 0.1 kb, 50 bp, 25 bp or 10 bp of naturally flanking nucleotide chromosomal DNA sequences of the microorganism from which the nucleic acid molecule is derived.

The alpha-olefin-associated enzyme, 1-alkene synthase, and/or phosphopantetheinyl transferase genes, as described herein, include nucleic acid molecules, for example, a polypeptide or RNA-encoding nucleic acid molecule, separated from another gene or other genes by intergenic DNA (for example, an intervening or spacer DNA which naturally flanks the gene and/or separates genes in the chromosomal DNA of the organism).

Nucleic acid molecules comprising a fragment of any one of the above-described nucleic acid sequences are also provided. These fragments preferably contain at least 20 contiguous nucleotides. More preferably the fragments of the nucleic acid sequences contain at least 25, 30, 35, 40, 45, 50, 60, 70, 80, 90, 100 or even more contiguous nucleotides.

In another embodiment, an isolated alpha-olefin-associated enzyme-encoding nucleic acid molecule hybridizes to all or a portion of a nucleic acid molecule having the nucleotide sequence set forth in SEQ ID NO:6 or hybridizes to all or a portion of a nucleic acid molecule having a nucleotide sequence that encodes a polypeptide having the amino acid sequence of SEQ ID NO: 7. Such hybridization conditions are known to those skilled in the art (see, for example, Current Protocols in Molecular Biology, Ausubel et al., eds., John Wiley & Sons, Inc. (1995); Molecular Cloning: A Laboratory Manual, Sambrook et al., Cold Spring Harbor Press, Cold Spring Harbor, N.Y. (1989)). In another embodiment, an isolated nucleic acid molecule comprises a nucleotide sequence that is complementary to a 1-alkene synthase-encoding nucleotide sequence as set forth herein.

In another embodiment, an isolated 1-alkene synthase-encoding nucleic acid molecule hybridizes to all or a portion of a nucleic acid molecule having the nucleotide sequence set forth in SEQ ID NO:2 or SEQ ID NO:4 or hybridizes to all or a portion of a nucleic acid molecule having a nucleotide sequence that encodes a polypeptide having the amino acid sequence of SEQ ID NO: 3 or SEQ ID NO:5. Such hybridization conditions are known to those skilled in the art (see, for example, Current Protocols in Molecular Biology, Ausubel et al., eds., John Wiley & Sons, Inc. (1995); Molecular Cloning: A Laboratory Manual, Sambrook et al., Cold Spring Harbor Press, Cold Spring Harbor, N.Y. (1989)). In another embodiment, an isolated nucleic acid molecule comprises a nucleotide sequence that is complementary to a polyketide synthase-encoding nucleotide sequence as set forth herein.

The nucleic acid sequence fragments display utility in a variety of systems and methods. For example, the fragments may be used as probes in various hybridization techniques. Depending on the method, the target nucleic acid sequences may be either DNA or RNA. The target nucleic acid sequences may be fractionated (e.g., by gel electrophoresis) prior to the hybridization, or the hybridization may be performed on samples in situ. One of skill in the art will appreciate that nucleic acid probes of known sequence find utility in determining chromosomal structure (e.g., by Southern blotting) and in measuring gene expression (e.g., by Northern blotting). In such experiments, the sequence fragments are preferably detectably labeled, so that their specific hybridization to target sequences can be detected and optionally quantified. One of skill in the art will appreciate that the nucleic acid fragments may be used in a wide variety of blotting techniques not specifically described herein.

It should also be appreciated that the nucleic acid sequence fragments disclosed herein also find utility as probes when immobilized on microarrays. Methods for creating microarrays by deposition and fixation of nucleic acids onto support substrates are well known in the art. Reviewed in DNA Microarrays: A Practical Approach (Practical Approach Series), Schena (ed.), Oxford University Press (1999) (ISBN: 0199637768); Nature Genet. 21(1)(suppl):1-60 (1999); Microarray Biochip: Tools and Technology, Schena (ed.), Eaton Publishing Company/BioTechniques Books Division (2000) (ISBN: 1881299376), the disclosures of which are incorporated herein by reference in their entireties. Analysis of, for example, gene expression using microarrays comprising nucleic acid sequence fragments, such as the nucleic acid sequence fragments disclosed herein, is a well-established utility for sequence fragments in the field of cell and molecular biology. Other uses for sequence fragments immobilized on microarrays are described in Gerhold et al., Trends Biochem. Sci. 24:168-173 (1999) and Zweiger, Trends Biotechnol. 17:429-436 (1999); DNA Microarrays: A Practical Approach (Practical Approach Series), Schena (ed.), Oxford University Press (1999) (ISBN: 0199637768); Nature Genet. 21(1)(suppl):1-60 (1999); Microarray Biochip: Tools and Technology, Schena (ed.), Eaton Publishing Company/BioTechniques Books Division (2000) (ISBN: 1881299376), the disclosures of each of which is incorporated herein by reference in its entirety.

In another embodiment, the invention provides isolated nucleic acid molecules encoding an alpha-olefin-associated enzyme which exhibits increased activity. In another embodiment, the invention provides isolated nucleic acid molecules encoding a 1-alkene synthase enzyme which exhibits increased activity.

As is well known in the art, enzyme activities are measured in various ways. For example, the pyrophosphorolysis of OMP may be followed spectroscopically. Grubmeyer et al., J. Biol. Chem. 268:20299-20304 (1993). Alternatively, the activity of the enzyme is followed using chromatographic techniques, such as by high performance liquid chromatography. Chung and Sloan, J. Chromatogr. 371:71-81 (1986). As another alternative the activity is indirectly measured by determining the levels of product made from the enzyme activity. More modern techniques include using gas chromatography linked to mass spectrometry (Niessen, W. M. A. (2001). Current practice of gas chromatography—mass spectrometry. New York, N.Y.: Marcel Dekker. (ISBN: 0824704738)). Additional modern techniques for identification of recombinant protein activity and products including liquid chromatography-mass spectrometry (LCMS), high performance liquid chromatography (HPLC), capillary electrophoresis, Matrix-Assisted Laser Desorption Ionization time of flight-mass spectrometry (MALDI-TOF MS), nuclear magnetic resonance (NMR), near-infrared (NIR) spectroscopy, viscometry (Knothe, G., R. O. Dunn, and M. O. Bagby. 1997. Biodiesel: The use of vegetable oils and their derivatives as alternative diesel fuels. Am. Chem. Soc. Symp. Series 666: 172-208), physical property-based methods, wet chemical methods, etc. are used to analyze the levels and the identity of the product produced by the organisms. Other methods and techniques may also be suitable for the measurement of enzyme activity, as would be known by one of skill in the art.

Another embodiment comprises mutant or chimeric 1-alkene synthase and/or alpha-olefin-associated enzyme nucleic acid molecules or genes. Typically, a mutant nucleic acid molecule or mutant gene is comprised of a nucleotide sequence that has at least one alteration including, but not limited to, a simple substitution, insertion or deletion. The polypeptide of said mutant can exhibit an activity that differs from the polypeptide encoded by the wild-type nucleic acid molecule or gene. Typically, a chimeric mutant polypeptide includes an entire domain derived from another polypeptide that is genetically engineered to be collinear with a corresponding domain. Preferably, a mutant nucleic acid molecule or mutant gene encodes a polypeptide having improved activity such as substrate affinity, substrate specificity, improved thermostability, activity at a different pH, or optimized codon usage for improved expression in a host cell.

### Vectors

The recombinant vector can be altered, modified or engineered to have different or a different quantity of nucleic acid sequences than in the derived or natural recombinant vector nucleic acid molecule. Preferably, the recombinant vector includes a gene or recombinant nucleic acid molecule operably linked to regulatory sequences including, but not limited to, promoter sequences, terminator sequences and/or artificial ribosome binding sites (RBSs), as defined herein.

Typically, a gene encoding alpha-olefin-associated enzyme is operably linked to regulatory sequence(s) in a manner which allows for the desired expression characteristics of the nucleotide sequence. Preferably, the gene encoding an alpha-olefin-associated enzyme is transcribed and translated into a gene product encoded by the nucleotide sequence when the recombinant nucleic acid molecule is included in a recombinant vector, as defined herein, and is introduced into a microorganism.

The regulatory sequence may be comprised of nucleic acid sequences which modulate, regulate or otherwise affect expression of other nucleic acid sequences. In one embodiment, a regulatory sequence can be in a similar or identical position and/or orientation relative to a nucleic acid sequence as observed in its natural state, e.g., in a native position and/or orientation. For example, a gene of interest can be included in a recombinant nucleic acid molecule or recombinant vector operably linked to a regulatory sequence which accompanies or is adjacent to the gene of interest in the natural host cell, or can be adjacent to a different gene in the natural host cell, or can be operably linked to a regulatory sequence from another organism. Regulatory sequences operably linked to a gene can be from other bacterial regulatory sequences, bacteriophage regulatory sequences and the like.

In one embodiment, a regulatory sequence is a sequence which has been modified, mutated, substituted, derivated, deleted, including sequences which are chemically synthesized. Preferably, regulatory sequences include promoters, enhancers, termination signals, anti-termination signals and other expression control elements that, for example, serve as sequences to which repressors or inducers bind or serve as or encode binding sites for transcriptional and/or translational regulatory polypeptides, for example, in the transcribed mRNA (see Sambrook, J., Fritsh, E. F., and Maniatis, T. Molecular Cloning: A Laboratory Manual. 2nd, ed, Cold Spring Harbor Laboratory, Cold Spring Harbor Laboratory Press, Cold Spring Harbor, N.Y., 1989). Regulatory sequences include promoters directing constitutive expression of a nucleotide sequence in a host cell, promoters directing inducible expression of a nucleotide sequence in a host cell and promoters which attenuate or repress expression of a nucleotide sequence in a host cell. Regulating expression of a gene of interest also can be done by removing or deleting regulatory sequences. For example, sequences involved in the negative regulation of transcription can be removed such that expression of a gene of interest is enhanced. In one embodiment, a recombinant nucleic acid molecule or recombinant vector includes a nucleic acid sequence or gene that encodes at least one bacterial alpha-olefin associated enzyme, wherein the gene encoding the enzyme(s) is operably linked to a promoter or promoter sequence. Preferably, promoters include native promoters, surrogate promoters and/or bacteriophage promoters.

In one embodiment, a promoter is associated with a biochemical housekeeping gene. In another embodiment, a promoter is a bacteriophage promoter. Other promoters include tef (the translational elongation factor (TEF) promoter) which promotes high level expression in Bacillus (e.g. Bacillus subtilis). Additional advantageous promoters, for example, for use in Gram positive microorganisms include, but are not limited to, the amyE promoter or phage SP02 promoters. Additional advantageous promoters, for example, for use in Gram negative microorganisms include, but are not limited to tac, trp, tet, trp-tet, lpp, lac, lpp-lac, laclq, T7, T5, T3, gal, trc, ara, SP6, λ-p_{R} or λ-p_{L}.

In another embodiment, a recombinant nucleic acid molecule or recombinant vector includes a transcription terminator sequence or sequences. Typically, terminator sequences refer to the regulatory sequences which serve to terminate transcription of a gene. Terminator sequences (or tandem transcription terminators) can further serve to stabilize mRNA (e.g., by adding structure to mRNA), for example, against nucleases.

In another embodiment, a recombinant nucleic acid molecule or recombinant vector has sequences allowing for detection of the vector containing sequences (i.e., detectable and/or selectable markers), for example, sequences that overcome auxotrophic mutations (e.g. ura3 or ilvE), fluorescent markers, and/or calorimetric markers (e.g., lacZ/β-galactosidase), and/or antibiotic resistance genes (e.g., gen, spec, bla or tet).

It is understood that any one of the polyketide synthase and/or alpha-olefin-associated enzyme encoding genes of the invention can be introduced into a vector also comprising one or more genes involved in the biosynthesis of 1-nonadecene from light, water and inorganic carbon.

Also provided are vectors, including expression vectors, which comprise the above nucleic acid molecules, as described further herein. In a first embodiment, the vectors include the isolated nucleic acid molecules described above. In an alternative embodiment, the vectors include the above-described nucleic acid molecules operably linked to one or more expression control sequences. The vectors of the instant invention may thus be used to express a polypeptide having an alpha-olefin associated enzyme and a 1-alkene synthase in a 1-nonadecene biosynthetic pathway.

Vectors useful for expression of nucleic acids in prokaryotes are well known in the art. A useful vector herein is plasmid pCDF Duet-1 that is available from Novagen. Another useful vector is the endogenous Synechococcus sp. PCC 7002 plasmid pAQ1 (Genbank accession number NC_{—}010476).

### Isolated Polypeptides

In one embodiment, polypeptides encoded by nucleic acid sequences are produced by recombinant DNA techniques and can be isolated from expression host cells by an appropriate purification scheme using standard polypeptide purification techniques. In another embodiment, polypeptides encoded by nucleic acid sequences are synthesized chemically using standard peptide synthesis techniques.

Included within the scope of the invention are alpha-olefin associated or gene products that are derived polypeptides or gene products encoded by naturally-occurring bacterial genes. Further, included within the inventive scope, are bacteria-derived polypeptides or gene products which differ from wild-type genes, including genes that have altered, inserted or deleted nucleic acids but which encode polypeptides substantially similar in structure and/or function to the wild-type alpha-olefin associated gene. Similar variants with respect to the 1-alkene synthase are also included within the scope of the invention.

For example, it is well understood that one of skill in the art can mutate (e.g., substitute) nucleic acids which, due to the degeneracy of the genetic code, encode for an identical amino acid as that encoded by the naturally-occurring gene. This may be desirable in order to improve the codon usage of a nucleic acid to be expressed in a particular organism. Moreover, it is well understood that one of skill in the art can mutate (e.g., substitute) nucleic acids which encode for conservative amino acid substitutions. It is further well understood that one of skill in the art can substitute, add or delete amino acids to a certain degree to improve upon or at least insubstantially affect the function and/or structure of a gene product (e.g., 1-alkene synthase activity) as compared with a naturally-occurring gene product, each instance of which is intended to be included within the scope of the invention. For example, the alpha-olefin associated enzyme activity, enzyme/substrate affinity, enzyme thermostability, and/or enzyme activity at various pHs can be unaffected or rationally altered and readily evaluated using the assays described herein.

In various aspects, isolated polypeptides (including muteins, allelic variants, fragments, derivatives, and analogs) encoded by the nucleic acid molecules are provided. In one embodiment, the isolated polypeptide comprises the polypeptide sequence corresponding to SEQ ID NO:7, SEQ ID NO:9, SEQ ID NO:11, SEQ ID NO:13, SEQ ID NO:15, or SEQ ID NO:17. In an alternative embodiment, the isolated polypeptide comprises a polypeptide sequence at least 50% identical to SEQ ID NO:7, SEQ ID NO:9, SEQ ID NO:11, SEQ ID NO:13, SEQ ID NO:15, or SEQ ID NO:17. Preferably the isolated polypeptide has 50%, 60%-70%, 70%-80%, 80%-90%, 90%-95%, 95%-98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or even higher identity to the sequences optimized for substrate affinity and/or substrate catalytic conversion rate.

According to other embodiments, isolated polypeptides comprising a fragment of the above-described polypeptide sequences are provided. These fragments preferably include at least 20 contiguous amino acids, more preferably at least 25, 30, 35, 40, 45, 50, 60, 70, 80, 90, 100 or even more contiguous amino acids.

The polypeptides also include fusions between the above-described polypeptide sequences and heterologous polypeptides. The heterologous sequences can, for example, include sequences designed to facilitate purification, e.g. histidine tags, and/or visualization of recombinantly-expressed proteins. Other non-limiting examples of protein fusions include those that permit display of the encoded protein on the surface of a phage or a cell, fusions to intrinsically fluorescent proteins, such as green fluorescent protein (GFP), and fusions to the IgG Fc region.

### Host Cell Transformants

In other aspects, host cells transformed with the nucleic acid molecules or vectors, and descendants thereof, are provided. In some embodiments, these cells carry the nucleic acid sequences on vectors which may be freely replicating vectors, e.g., pAQ1, pAQ3, pAQ4, pAQ5, pAQ6, and pAQ7. In other embodiments, the nucleic acids have been integrated into the genome of the host cells.

The host cell encoding alpha-olefin-associated enzyme can be a host cell lacking an endogenous alpha-olefin-associated enzyme gene or a host with an endogenous alpha-olefin-associated enzyme gene. The host cell can be engineered to express a recombinant alpha-olefin-associated enzyme in addition to its endogenous alpha-olefin-associated enzyme gene, and/or the host cell can be modified such that its endogenous alpha-olefin-associated enzyme gene is overexpressed (e.g., by promoter swapping or by increasing read-through from an upstream promoter).

In a preferred embodiment, the host cell comprises one or more recombinant nucleic acids encoding a alpha-olefin-associated enzyme (e.g., SEQ ID NO:6).

In an alternative embodiment, the host cells can be mutated by recombination with a disruption, deletion or mutation of the isolated nucleic acid so that the activity of the alpha-olefin-associated enzyme is reduced or eliminated compared to a host cell lacking the mutation.

In another embodiment, the host cell containing a 1-alkene synthase and alpha-olefin-associated enzyme is suitable for producing 1-nonadecene or 1-octadiene. In a particular embodiment, the host cell is a recombinant host cell that produces 1-nonadecene comprising a heterologous nucleic acid encoding a nucleic acid of SEQ ID NO:6.

In certain aspects, methods for expressing a polypeptide under suitable culture conditions and choice of host cell line for optimal enzyme expression, activity and stability (codon usage, salinity, pH, temperature, etc.) are provided.

In another aspect, the invention provides methods for producing 1-alkenes (e.g., 1-nonadecene, 1-octadecene, and/or other long-chain 1-alkenes) by culturing a host cell under conditions in which the alpha-olefin associated enzyme is expressed at sufficient levels to provide a measurable increase in the quantity of production of the -alkene of interest (e.g., 1-nonadecene, 1-octadecene, etc). In a related embodiment, methods for producing 1-alkenes are carried out by contacting a cell lysate obtained from the above host cell under conditions in which the 1-alkenes are produced from light, water and inorganic carbon. Accordingly, the invention provides enzyme extracts having improved alpha-olefin-associated enzyme activity, and having, for example, thermal stability, activity at various pH, and/or superior substrate affinity or specificity.

### Selected or Engineered Microorganisms for the Production of Carbon-Based Products of Interest

Microorganism: Includes prokaryotic and eukaryotic microbial species from the Domains Archaea, Bacteria and Eucarya, the latter including yeast and filamentous fungi, protozoa, algae, or higher Protista. The terms “microbial cells” and “microbes” are used interchangeably with the term microorganism.

A variety of host organisms can be transformed to produce 1-alkenes. Photoautotrophic organisms include eukaryotic plants and algae, as well as prokaryotic cyanobacteria, green-sulfur bacteria, green non-sulfur bacteria, purple sulfur bacteria, and purple non-sulfur bacteria.

Host cells can be a Gram-negative bacterial cell or a Gram-positive bacterial cell. A Gram-negative host cell of the invention can be, e.g., Gluconobacter, Rhizobium, Bradyrhizobium, Alcaligenes, Rhodobacter, Rhodococcus. Azospirillum, Rhodospirillum, Sphingomonas, Burkholderia, Desuifomonas, Geospirillum, Succinomonas, Aeromonas, Shewanella, Halochromatium, Citrobacter, Escherichia, Klebsiella, Zymomonas Zymobacter, or Acetobacter. A Gram-positive host cell of the invention can be, e.g., Fibrobacter, Acidobacter, Bacteroides, Sphingobacterium, Actinomyces, Corynebacterium, Nocardia, Rhodococcus, Propionibacterium, Bifidobacterium, Bacillus, Geobacillus, Paenibacillus, Sulfobacillus, Clostridium, Anaerobacter, Eubacterium, Streptococcus, Lactobacillus, Leuconostoc, Enterococcus, Lactococcus, Thermobifida, Cellulomonas, or Sarcina.

Extremophiles are also contemplated as suitable organisms. Such organisms withstand various environmental parameters such as temperature, radiation, pressure, gravity, vacuum, desiccation, salinity, pH, oxygen tension, and chemicals. They include hyperthermophiles, which grow at or above 80° C. such as Pyrolobus fumarii; thermophiles, which grow between 60-80° C. such as Synechococcus lividis; mesophiles, which grow between 15-60° C. and psychrophiles, which grow at or below 15° C. such as Psychrobacter and some insects. Radiation tolerant organisms include Deinococcus radiodurans. Pressure tolerant organisms include piezophiles or barophiles which tolerate pressure of 130 MPa. Hypergravity (e.g., >1 g) hypogravity (e.g., <1 g) tolerant organisms are also contemplated. Vacuum tolerant organisms include tardigrades, insects, microbes and seeds. Dessicant tolerant and anhydrobiotic organisms include xerophiles such as Artemia salina; nematodes, microbes, fungi and lichens. Salt tolerant organisms include halophiles (e.g., 2-5 M NaCl) Halobacteriacea and Dunaliella salina. pH tolerant organisms include alkaliphiles such as Natronobacterium, Bacillus firmus OF4, Spirulina spp. (e.g., pH>9) and acidophiles such as Cyanidium caldarium, Ferroplasma sp. (e.g., low pH). Anaerobes, which cannot tolerate O₂ such as Methanococcus jannaschii; microaerophils, which tolerate some O₂ such as Clostridium and aerobes, which require O₂ are also contemplated. Gas tolerant organisms, which tolerate pure CO₂ include Cyanidium caldarium and metal tolerant organisms include metalotolerants such as Ferroplasma acidarmanus (e.g., Cu, As, Cd, Zn), Ralstonia sp. CH34 (e.g., Zn, Co, Cd, Hg, Pb). Gross, Michael. Life on the Edge: Amazing Creatures Thriving in Extreme Environments. New York: Plenum (1998) and Seckbach, J. “Search for Life in the Universe with Terrestrial Microbes Which Thrive Under Extreme Conditions.” In Cristiano Batalli Cosmovici, Stuart Bowyer, and Dan Wertheimer, eds., Astronomical and Biochemical Origins and the Search for Life in the Universe, p. 511. Milan: Editrice Compositori (1997).

Plants include but are not limited to the following genera: Arabidopsis, Beta, Glycine, Jatropha, Miscanthus, Panicum, Phalaris, Populus, Saccharum, Salix, Simmondsia and Zea.

Algae and cyanobacteria include but are not limited to the following genera: Acanthoceras, Acanthococcus, Acaryochloris, Achnanthes, Achnanthidium, Actinastrum, Actinochloris, Actinocyclus, Actinotaenium, Amphichrysis, Amphidinium, Amphikrikos, Amphipleura, Amphiprora, Amphithrix, Amphora, Anabaena, Anabaenopsis, Aneumastus, Ankistrodesmus, Ankyra, Anomoeoneis, Apatococcus, Aphanizomenon, Aphanocapsa, Aphanochaete, Aphanothece, Apiocystis, Apistonema, Arthrodesmus, Artherospira, Ascochloris, Asterionella, Asterococcus, Audouinella, Aulacoseira, Bacillaria, Balbiania, Bambusina, Bangia, Basichlamys, Batrachospermum, Binuclearia, Bitrichia, Blidingia, Botrdiopsis, Botrydium, Botryococcus, Botryosphaerella, Brachiomonas, Brachysira, Brachytrichia, Brebissonia, Bulbochaete, Bumilleria, Bumilleriopsis, Caloneis, Calothrix, Campylodiscus, Capsosiphon, Carteria, Catena, Cavinula, Centritractus, Centronella, Ceratium, Chaetoceros, Chaetochloris, Chaetomorpha, Chaetonella, Chaetonema, Chaetopeltis, Chaetophora, Chaetosphaeridium, Chamaesiphon, Chara, Characiochloris, Characiopsis, Characium, Charales, Chilomonas, Chlainomonas, Chlamydoblepharis, Chlamydocapsa, Chlamydomonas, Chlamydomonopsis, Chlamydomyxa, Chlamydonephris, Chlorangiella, Chlorangiopsis, Chlorella, Chlorobotrys, Chlorobrachis, Chlorochytrium, Chlorococcum, Chlorogloea, Chlorogloeopsis, Chlorogonium, Chlorolobion, Chloromonas, Chlorophysema, Chlorophyta, Chlorosaccus, Chlorosarcina, Choricystis, Chromophyton, Chromulina, Chroococcidiopsis, Chroococcus, Chroodactylon, Chroomonas, Chroothece, Chrysamoeba, Chrysapsis, Chrysidiastrum, Chrysocapsa, Chrysocapsella, Chrysochaete, Chrysochromulina, Chrysococcus, Chrysocrinus, Chrysolepidomonas, Chrysolykos, Chrysonebula, Chrysophyta, Chrysopyxis, Chrysosaccus, Chrysophaerella, Chrysostephanosphaera, Clodophora, Clastidium, Closteriopsis, Closterium, Coccomyxa, Cocconeis, Coelastrella, Coelastrum, Coelosphaerium, Coenochloris, Coenococcus, Coenocystis, Colacium, Coleochaete, Collodictyon, Compsogonopsis, Compsopogon, Conjugatophyta, Conochaete, Coronastrum, Cosmarium, Cosmioneis, Cosmocladium, Crateriportula, Craticula, Crinalium, Crucigenia, Crucigeniella, Cryptoaulax, Cryptomonas, Cryptophyta, Ctenophora, Cyanodictyon, Cyanonephron, Cyanophora, Cyanophyta, Cyanothece, Cyanothomonas, Cyclonexis, Cyclostephanos, Cyclotella, Cylindrocapsa, Cylindrocystis, Cylindrospermum, Cylindrotheca, Cymatopleura, Cymbella, Cymbellonitzschia, Cystodinium Dactylococcopsis, Debarya, Denticula, Dermatochrysis, Dermocarpa, Dermocarpella, Desmatractum, Desmidium, Desmococcus, Desmonema, Desmosiphon, Diacanthos, Diacronema, Diadesmis, Diatoma, Diatomella, Dicellula, Dichothrix, Dichotomococcus, Dicranochaete, Dictyochloris, Dictyococcus, Dictyosphaerium, Didymocystis, Didymogenes, Didymosphenia, Dilabifilum, Dimorphococcus, Dinobryon, Dinococcus, Diplochloris, Diploneis, Diplostauron, Distrionella, Docidium, Draparnaldia, Dunaliella, Dysmorphococcus, Ecballocystis, Elakatothrix, Ellerbeckia, Encyonema, Enteromorpha, Entocladia, Entomoneis, Entophysalis, Epichrysis, Epipyxis, Epithemia, Eremosphaera, Euastropsis, Euastrum, Eucapsis, Eucocconeis, Eudorina, Euglena, Euglenophyta, Eunotia, Eustigmatophyta, Eutreptia, Fallacia, Fischerella, Fragilaria, Fragilariforma, Franceia, Frustulia, Curcilla, Geminella, Genicularia, Glaucocystis, Glaucophyta, Glenodiniopsis, Glenodinium, Gloeocapsa, Gloeochaete, Gloeochrysis, Gloeococcus, Gloeocystis, Gloeodendron, Gloeomonas, Gloeoplax, Gloeothece, Gloeotila, Gloeotrichia, Gloiodictyon, Golenkinia, Golenkiniopsis, Gomontia, Gomphocymbella, Gomphonema, Gomphosphaeria, Gonatozygon, Gongrosia, Gongrosira, Goniochloris, Gonium, Gonyostomum, Granulochloris, Granulocystopsis, Groenbladia, Gymnodinium, Gymnozyga, Gyrosigma, Haematococcus, Hafniomonas, Hallassia, Hammatoidea, Hannaea, Hantzschia, Hapalosiphon, Haplotaenium, Haptophyta, Haslea, Hemidinium, Hemitoma, Heribaudiella, Heteromastix, Heterothrix, Hibberdia, Hildenbrandia, Hillea, Holopedium, Homoeothrix, Hormanthonema, Hormotila, Hyalobrachion, Hyalocardium, Hyalodiscus, Hyalogonium, Hyalotheca, Hydrianum, Hydrococcus, Hydrocoleum, Hydrocoryne, Hydrodictyon, Hydrosera, Hydrurus, Hyella, Hymenomonas, Isthmochloron, Johannesbaptistia, Juranyiella, Karayevia, Kathablepharis, Katodinium, Kephyrion, Keratococcus, Kirchneriella, Klebsormidium, Kolbesia, Koliella, Komarekia, Korshikoviella, Kraskella, Lagerheimia, Lagynion, Lamprothamnium, Lemanea, Lepocinclis, Leptosira, Lobococcus, Lobocystis, Lobomonas, Luticola, Lyngbya, Malleochloris, Mallomonas, Mantoniella, Marssoniella, Martyana, Mastigocoleus, Gastogloia, Melosira, Merismopedia, Mesostigma, Mesotaenium, Micractinium, Micrasterias, Microchaete, Microcoleus, Microcystis, Microglena, Micromonas, Microspora, Microthamnion, Mischococcus, Monochrysis, Monodus, Monomastix, Monoraphidium, Monostroma, Mougeotia, Mougeotiopsis, Myochloris, Myromecia, Myxosarcina, Naegeliella, Nannochloris, Nautococcus, Navicula, Neglectella, Neidium, Nephroclamys, Nephrocytium, Nephrodiella, Nephroselmis, Netrium, Nitella, Nitellopsis, Nitzschia, Nodularia, Nostoc, Ochromonas, Oedogonium, Oligochaetophora, Onychonema, Oocardium, Oocystis, Opephora, Ophiocytium, Orthoseira, Oscillatoria, Oxyneis, Pachycladella, Palmella, Palmodictyon, Pnadorina, Pannus, Paralia, Pascherina, Paulschulzia, Pediastrum, Pedinella, Pedinomonas, Pedinopera, Pelagodictyon, Penium, Peranema, Peridiniopsis, Peridinium, Peronia, Petroneis, Phacotus, Phacus, Phaeaster, Phaeodermatium, Phaeophyta, Phaeosphaera, Phaeothamnion, Phormidium, Phycopeltis, Phyllariochloris, Phyllocardium, Phyllomitas, Pinnularia, Pitophora, Placoneis, Planctonema, Planktosphaeria, Planothidium, Plectonema, Pleodorina, Pleurastrum, Pleurocapsa, Pleurocladia, Pleurodiscus, Pleurosigma, Pleurosira, Pleurotaenium, Pocillomonas, Podohedra, Polyblepharides, Polychaetophora, Polyedriella, Polyedriopsis, Polygoniochloris, Polyepidomonas, Polytaenia, Polytoma, Polytomella, Porphyridium, Posteriochromonas, Prasinochloris, Prasinocladus, Prasinophyta, Prasiola, Prochlorphyta, Prochlorothrix, Protoderma, Protosiphon, Provasoliella, Prymnesium, Psammodictyon, Psammothidium, Pseudanabaena, Pseudenoclonium, Psuedocarteria, Pseudochate, Pseudocharacium, Pseudococcomyxa, Pseudodictyosphaerium, Pseudokephyrion, Pseudoncobyrsa, Pseudoquadrigula, Pseudosphaerocystis, Pseudostaurastrum, Pseudostaurosira, Pseudotetrastrum, Pteromonas, Punctastruata, Pyramichlamys, Pyramimonas, Pyrrophyta, Quadrichloris, Quadricoccus, Quadrigula, Radiococcus, Radiofilum, Raphidiopsis, Raphidocelis, Raphidonema, Raphidophyta, Peimeria, Rhabdoderma, Rhabdomonas, Rhizoclonium, Rhodomonas, Rhodophyta, Rhoicosphenia, Rhopalodia, Rivularia, Rosenvingiella, Rossithidium, Roya, Scenedesmus, Scherffelia, Schizochlamydella, Schizochlamys, Schizomeris, Schizothrix, Schroederia, Scolioneis, Scotiella, Scotiellopsis, Scourfieldia, Scytonema, Selenastrum, Selenochloris, Sellaphora, Semiorbis, Siderocelis, Diderocystopsis, Dimonsenia, Siphononema, Sirocladium, Sirogonium, Skeletonema, Sorastrum, Spermatozopsis, Sphaerellocystis, Sphaerellopsis, Sphaerodinium, Sphaeroplea, Sphaerozosma, Spiniferomonas, Spirogyra, Spirotaenia, Spirulina, Spondylomorum, Spondylosium, Sporotetras, Spumella, Staurastrum, Stauerodesmus, Stauroneis, Staurosira, Staurosirella, Stenopterobia, Stephanocostis, Stephanodiscus, Stephanoporos, Stephanosphaera, Stichococcus, Stichogloea, Stigeoclonium, Stigonema, Stipitococcus, Stokesiella, Strombomonas, Stylochrysalis, Stylodinium, Styloyxis, Stylosphaeridium, Surirella, Sykidion, Symploca, Synechococcus, Synechocystis, Synedra, Synochromonas, Synura, Tabellaria, Tabularia, Teilingia, Temnogametum, Tetmemorus, Tetrachlorella, Tetracyclus, Tetradesmus, Tetraedriella, Tetraedron, Tetraselmis, Tetraspora, Tetrastrum, Thalassiosira, Thamniochaete, Thorakochloris, Thorea, Tolypella, Tolypothrix, Trachelomonas, Trachydiscus, Trebouxia, Trentepholia, Treubaria, Tribonema, Trichodesmium, Trichodiscus, Trochiscia, Tryblionella, Ulothrix, Uroglena, Uronema, Urosolenia, Urospora, Uva, Vacuolaria, Vaucheria, Volvox, Volvulina, Westella, Woloszynskia, Xanthidium, Xanthophyta, Xenococcus, Zygnema, Zygnemopsis, and Zygonium.

Green non-sulfur bacteria include but are not limited to the following genera: Chloroflexus, Chloronema, Oscillochloris, Heliothrix, Herpetosiphon, Roseiflexus, and Thermomicrobium.

Green sulfur bacteria include but are not limited to the following genera: Chlorobium, Clathrochloris, and Prosthecochloris.

Purple sulfur bacteria include but are not limited to the following genera: Allochromatium, Chromatium, Halochromatium, Isochromatium, Marichromatium, Rhodovulum, Thermochromatium, Thiocapsa, Thiorhodococcus, and Thiocystis,

Purple non-sulfur bacteria include but are not limited to the following genera: Phaeospirillum, Rhodobaca, Rhodobacter, Rhodomicrobium, Rhodopila, Rhodopseudomonas, Rhodothalassium, Rhodospirillum, Rodovibrio, and Roseospira.

Aerobic chemolithotrophic bacteria include but are not limited to nitrifying bacteria such as Nitrobacteraceae sp., Nitrobacter sp., Nitrospina sp., Nitrococcus sp., Nitrospira sp., Nitrosomonas sp., Nitrosococcus sp., Nitrosospira sp., Nitrosolobus sp., Nitrosovibrio sp.; colorless sulfur bacteria such as, Thiovulum sp., Thiobacillus sp., Thiomicrospira sp., Thiosphaera sp., Thermothrix sp.; obligately chemolithotrophic hydrogen bacteria such as Hydrogenobacter sp., iron and manganese-oxidizing and/or depositing bacteria such as Siderococcus sp., and magnetotactic bacteria such as Aquaspirillum sp.

Archaeobacteria include but are not limited to methanogenic archaeobacteria such as Methanobacterium sp., Methanobrevibacter sp., Methanothermus sp., Methanococcus sp., Methanomicrobium sp., Methanospirillum sp., Methanogenium sp., Methanosarcina sp., Methanolobus sp., Methanothrix sp., Methanococcoides sp., Methanoplanus sp.; extremely thermophilic sulfur-metabolizers such as Thermoproteus sp., Pyrodictium sp., Sulfolobus sp., Acidianus sp. and other microorganisms such as, Bacillus subtilis, Saccharomyces cerevisiae, Streptomyces sp., Ralstonia sp., Rhodococcus sp., Corynebacteria sp., Brevibacteria sp., Mycobacteria sp., and oleaginous yeast.

In preferred embodiments the parental photoautotrophic organism can be transformed with a gene encoding an alpha-olefin-associated enzyme.

Preferred organisms for HyperPhotosynthetic conversion include: Arabidopsis thaliana, Panicum virgatum, Miscanthus giganteus, and Zea mays (plants), Botryococcus braunii, Chlamydomonas reinhardtii and Dunaliela salina (algae), Synechococcus sp PCC 7002, Synechococcus sp. PCC 7942, Synechocystis sp. PCC 6803, and Thermosynechococcus elongatus BP-1 (cyanobacteria), Chlorobium tepidum (green sulfur bacteria), Chloroflexus auranticus (green non-sulfur bacteria), Chromatium tepidum and Chromatium vinosum (purple sulfur bacteria), Rhodospirillum rubrum, Rhodobacter capsulatus, and Rhodopseudomonas palusris (purple non-sulfur bacteria).

Yet other suitable organisms include synthetic cells or cells produced by synthetic genomes as described in Venter et al. US Pat. Pub. No. 2007/0264688, and cell-like systems or synthetic cells as described in Glass et al. US Pat. Pub. No. 2007/0269862.

Still, other suitable organisms include microorganisms that can be engineered to fix carbon dioxide, e.g., bacteria such as Escherichia coli, Acetobacter aceti, Bacillus subtilis, yeast and fungi such as Clostridium ljungdahlii, Clostridium thermocellum, Penicillium chrysogenum, Pichia pastoris, Saccharomyces cerevisiae, Schizosaccharomyces pombe, Pseudomonas fluorescens, or Zymomonas mobilis.

A common theme in selecting or engineering a suitable organism is autotrophic fixation of CO₂ to products. This would cover photosynthesis and methanogenesis. Acetogenesis, encompassing the three types of CO₂ fixation; Calvin cycle, acetyl CoA pathway and reductive TCA pathway is also covered. The capability to use carbon dioxide as the sole source of cell carbon (autotrophy) is found in almost all major groups of prokaryotes. The CO₂ fixation pathways differ between groups, and there is no clear distribution pattern of the four presently-known autotrophic pathways. Fuchs, G. 1989. Alternative pathways of autotrophic CO₂ fixation, p. 365-382. In H. G. Schlegel, and B. Bowien (ed.), Autotrophic bacteria. Springer-Verlag, Berlin, Germany. The reductive pentose phosphate cycle (Calvin-Bassham-Benson cycle) represents the CO₂ fixation pathway in many aerobic autotrophic bacteria, for example, cyanobacteria.

### Gene Integration and Propagation

The aoa gene can be propagated by insertion into the host cell genome. Integration into the genome of the host cell is optionally done at particular loci to impair or disable unwanted gene products or metabolic pathways.

In another embodiment is described the integration of a 1-alkene synthase gene and/or an aoa gene in the 1-alkene synthesis pathway into a plasmid. The plasmid can express one or more genes, optionally an operon including one or more genes, preferably one or more genes involved in the synthesis of 1-alkenes, or more preferably one or more genes of a related metabolic pathway that feeds into the biosynthetic pathway for 1-alkenes.

Yet another embodiment provides a method of integrating one or more aoa genes into an expression vector.

### Antibodies

In another aspect, provided herein are isolated antibodies, including fragments and derivatives thereof that bind specifically to the isolated polypeptides and polypeptide fragments or to one or more of the polypeptides encoded by the isolated nucleic acids. The antibodies may be specific for linear epitopes, discontinuous epitopes or conformational epitopes of such polypeptides or polypeptide fragments, either as present on the polypeptide in its native conformation or, in some cases, as present on the polypeptides as denatured, as, e.g., by solubilization in SDS. Among the useful antibody fragments are Fab, Fab′, Fv, F(ab′)₂, and single chain Fv fragments.

By “bind specifically” and “specific binding” is here intended the ability of the antibody to bind to a first molecular species in preference to binding to other molecular species with which the antibody and first molecular species are admixed. An antibody is said specifically to “recognize” a first molecular species when it can bind specifically to that first molecular species.

As is well known in the art, the degree to which an antibody can discriminate as among molecular species in a mixture will depend, in part, upon the conformational relatedness of the species in the mixture; typically, the antibodies will discriminate over adventitious binding to unrelated polypeptides by at least two-fold, more typically by at least 5-fold, typically by more than 10-fold, 25-fold, 50-fold, 75-fold, and often by more than 100-fold, and on occasion by more than 500-fold or 1000-fold.

Typically, the affinity or avidity of an antibody (or antibody multimer, as in the case of an IgM pentamer) for a polypeptide or polypeptide fragment will be at least about 1×10⁻⁶ M, typically at least about 5×10⁻⁷ M, usefully at least about 1×10⁻⁷ M, with affinities and avidities of 1×10⁻⁸ M, 5×10⁻⁹ M, 1×10⁻¹⁰ M and even stronger proving especially useful.

The isolated antibodies may be naturally-occurring forms, such as IgG, IgM, IgD, IgE, and IgA, from any mammalian species. For example, antibodies are usefully obtained from species including rodents-typically mouse, but also rat, guinea pig, and hamster-lagomorphs, typically rabbits, and also larger mammals, such as sheep, goats, cows, and horses. The animal is typically affirmatively immunized, according to standard immunization protocols, with the polypeptide or polypeptide fragment.

Virtually all fragments of 8 or more contiguous amino acids of the polypeptides may be used effectively as immunogens when conjugated to a carrier, typically a protein such as bovine thyroglobulin, keyhole limpet hemocyanin, or bovine serum albumin, conveniently using a bifunctional linker. Immunogenicity may also be conferred by fusion of the polypeptide and polypeptide fragments to other moieties. For example, peptides can be produced by solid phase synthesis on a branched polylysine core matrix; these multiple antigenic peptides (MAPs) provide high purity, increased avidity, accurate chemical definition and improved safety in vaccine development. See, e.g., Tam et al., Proc. Natl. Acad. Sci. USA 85:5409-5413 (1988); Posnett et al., J. Biol. Chem. 263, 1719-1725 (1988).

Protocols for immunization are well-established in the art. Such protocols often include multiple immunizations, either with or without adjuvants such as Freund's complete adjuvant and Freund's incomplete adjuvant. Antibodies may be polyclonal or monoclonal, with polyclonal antibodies having certain advantages in immunohistochemical detection of the proteins and monoclonal antibodies having advantages in identifying and distinguishing particular epitopes of the proteins. Following immunization, the antibodies may be produced using any art-accepted technique. Host cells for recombinant antibody production—either whole antibodies, antibody fragments, or antibody derivatives—can be prokaryotic or eukaryotic. Prokaryotic hosts are particularly useful for producing phage displayed antibodies, as is well known in the art. Eukaryotic cells, including mammalian, insect, plant and fungal cells are also useful for expression of the antibodies, antibody fragments, and antibody derivatives. Antibodies can also be prepared by cell free translation.

The isolated antibodies, including fragments and derivatives thereof, can usefully be labeled. It is, therefore, another aspect to provide labeled antibodies that bind specifically to one or more of the polypeptides and polypeptide fragments. The choice of label depends, in part, upon the desired use. In some cases, the antibodies may usefully be labeled with an enzyme. Alternatively, the antibodies may be labeled with colloidal gold or with a fluorophore. For secondary detection using labeled avidin, streptavidin, captavidin or neutravidin, the antibodies may usefully be labeled with biotin. When the antibodies are used, e.g., for Western blotting applications, they may usefully be labeled with radioisotopes, such as ³³P, ³²P, ³⁵S, ³H and ¹²⁵I. As would be understood, use of the labels described above is not restricted to any particular application.

### Methods for Designing Protein Variants

Increased 1-alkene production can be achieved through the expression and optimization of the 1-alkene synthase, the 1-alkene synthesis pathway, and the alpha-olefin-associated enzyme in organisms well suited for modern genetic engineering techniques, i.e., those that rapidly grow, are capable of thriving on inexpensive food resources and from which isolation of a desired product is easily and inexpensively achieved. To increase the rate of production of 1-alkenes it would be advantageous to design and select variants of the enzymes, including but not limited to, variants optimized for substrate affinity, substrate specificity, substrate catalytic conversion rate, improved thermostability, activity at a different pH and/or optimized codon usage for improved expression in a host cell. See, for example, amino acid changes correlated to alterations in the catalytic rate while maintaining similar affinities (R L Zheng and R G Kemp, J. Biol. Chem. (1994) Vol. 269:18475-18479) or amino acid changes correlated with changes in the stability of the transition state that affect catalytic turnover (MA Phillips, et al., J. Biol. Chem., (1990) Vol. 265:20692-20698). It would be another advantage to design and select for enzymes altered to have substantially decreased reverse reaction activity in which enzyme-substrate products would be the result of energetically unfavorable bond formation or molecular re-configuration of the substrate, and have improved forward reaction activity in which enzyme-substrate products would be the result of energetically favorable molecular bond reduction or molecular re-configuration.

Accordingly, one method for the design of improved polyketide synthase proteins for synthesing 1-nonadecene utilizes computational and bioinformatic analysis to design and select for advantageous changes in primary amino acid sequences encoding ethanologenic enzyme activity. Computational methods and bioinformatics provide tractable alternatives for rational design of protein structure and function. Recently, algorithms analyzing protein structure for biophysical character (for example, motional dynamics and total energy or Gibbs Free Energy evaluations) have become a commercially feasible methodology supplementing protein sequence analysis data that assess homology, identity and/or degree of sequence and domain conservation to improve upon or design the desirable qualities of a protein (Rosetta++, University of Washington). For example, an in silico redesign of the endonuclease I-MsoI was based on computational evaluation of biophysical parameters of rationally selected changes to the primary amino acid sequence. Researchers were able to maintain wild-type binding selectivity and affinity yet improve the catalytic turnover by four orders of magnitude (Ashworth, et al., Nature (2006) vol. 441:656-659).

In one embodiment, polypeptide sequences or related homologues in a complex with a substrate are obtained from the Protein Data Bank (PDB; H M Berman, et al., Nucleic Acids Research (2000) vol. 28:235-242) for computational analysis on steady state and/or changes in Gibbs free energy relative to the wild type protein. Substitutions of one amino acid residue for another are accomplished in silico interactively as a means for identifying specific residue substitutions that optimize structural or catalytic contacts between the protein and substrate using standard software programs for viewing molecules as is well known to those skilled in the art. To the extent that in silico structures for the polypeptides (and homologues) described herein are available through the PDB, those structures can be used to rationally design modified proteins with desired (typically, improved) activities. Specific amino acid substitutions are rationally chosen based on substituted residue characteristics that optimize, for example, Van der Waal's interactions, hydrophobicity, hydrophilicity, steric non-interferences, pH-dependent electrostatics and related chemical interactions. The overall energetic change of the substitution protein model when unbound and bound to its substrate is calculated and assessed by one having skill in the art to be evaluated for the change in free energy for correlations to overall structural stability (e.g., Meiler, J. and D. Baker, Proteins (2006) 65:538-548). In addition, such computational methods provide a means for accurately predicting quaternary protein structure interactions such that in silico modifications are predictive or determinative of overall multimeric structural stability (Wollacott, A M, et al., Protein Science (2007) 16:165-175; Joachimiak, L A, et al., J. Mol. Biol. (2006) 361:195-208).

Preferably, a rational design change to the primary structure of Aoa protein sequences minimally alters the Gibbs free energy state of the unbound polypeptide and maintains a folded, functional and similar wild-type enzyme structure. More preferably a lower computational total free energy change of the protein sequence is achieved to indicate the potential for optimized enzyme structural stability.

Although lower free energy of a protein structure relative to the wild type structure is an indicator of thermodynamic stability, the positive correlation of increased thermal stability to optimized function does not always exist. Therefore, preferably, optimal catalytic contacts between the modified Aoa protein structure and the substrate are achieved with a concomitant predicted favorable change in total free energy of the catabolic reaction, for example by rationally designing Aoa protein/substrate interactions that stabilize the transition state of the enzymatic reaction while maintaining a similar or favorable change in free energy of the unbound Aoa protein for a desired environment in which a host cell expresses the mutant Aoa protein. Even more preferably, rationally selected amino acid changes result in a substantially decreased Aoa enzyme's anabolic protein/substrate reaction or increase the Aoa enzyme's catabolic protein/substrate reaction. In a further embodiment any and/or all aoa sequences are expression optimized for the specific expression host cell.

### Methods for Generating Protein Variants

Several methods well known to those with skill in the art are available to generate random nucleotide sequence variants for a corresponding polypeptide sequence using the Polymerase Chain Reaction (“PCR”) (U.S. Pat. No. 4,683,202). One embodiment is the generation of aoa gene variants using the method of error prone PCR. (R. Cadwell and G. Joyce, PCR Meth. Appl. (1991) Vol. 2:28-33; Leung, et al., Technique (1989) Vol. 1:11-15). Error prone PCR is achieved by the establishment of a chemical environment during the PCR experiment that causes an increase in unfaithful replication of a parent copy of DNA sought to be replicated. For example, increasing the manganese or magnesium ion content of the chemical admixture used in the PCR experiment, very low annealing temperatures, varying the balance among di-deoxy nucleotides added, starting with a low population of parent DNA templates or using polymerases designed to have increased inefficiencies in accurate DNA replication all result in nucleotide changes in progeny DNA sequences during the PCR replication process. The resultant mutant DNA sequences are genetically engineered into an appropriate vector to be expressed in a host cell and analyzed to screen and select for the desired effect on whole cell production of a product or process of interest. In one embodiment, random mutagenesis of the Aoa-encoding nucleotide sequences is generated through error prone PCR using techniques well known to one skilled in the art. Resultant nucleotide sequences are analyzed for structural and functional attributes through clonal screening assays and other methods as described herein.

Another embodiment is generating a specifically desired protein mutant using site-directed mutagenesis. For example, with overlap extension (An, et al., Appl. Microbiol. Biotech. (2005) vol. 68(6):774-778) or mega-primer PCR (E. Burke and S. Batik, Methods Mol. Bio. (2003) vol 226:525-532) one can use nucleotide primers that have been altered at corresponding codon positions in the parent nucleotide to yield DNA progeny sequences containing the desired mutation. Alternatively, one can use cassette mutagenesis (Kegler-Ebo, et al., Nucleic Acids Res. (1994) vol. 22(9):1593-1599) as is commonly known by one skilled in the art.

In one aspect, using site-directed mutagenesis and cassette mutagenesis, all possible positions in SEQ ID NO: 7 are changed to a proline, transformed into a suitable high expression vector and expressed at high levels in a suitable expression host cell. Purified aliquots at concentrations necessary for the appropriate biophysical analytical technique are obtained by methods as known to those with skill in the art (P. Rellos and R. K. Scopes, Prot. Exp. Purific. (1994) Vol. 5:270-277) and evaluated for increased thermostability.

Another embodiment is to select for a polypeptide variant for expression in a recipient host cell by comparing a first nucleic acid sequence encoding the polypeptide with the nucleic acid sequence of a second, related nucleic acid sequence encoding a polypeptide having more desirable qualities, and altering at least one codon of the first nucleic acid sequence to have identity with the corresponding codon of the second nucleic acid sequence, such that improved polypeptide activity, substrate specificity, substrate affinity, substrate catalytic conversion rate, improved thermostability, activity at a different pH and/or optimized codon usage for expression and/or structure of the altered polypeptide is achieved in the host cell.

In yet another embodiment, all amino acid residue variations are encoded at any desired, specified nucleotide codon position using such methods as site saturation mutagenesis (Meyers, et al., Science (1985) Vol. 229:242-247; Derbyshire, et al., Gene (1986) Vol. 46:145-152; U.S. Pat. No. 6,171,820). Whole gene site saturation mutagenesis (K. Kretz, et al., Meth. Enzym. (2004) Vol. 388:3-11) is preferred wherein all amino acid residue variations are encoded at every nucleotide codon position. Both methods yield a population of protein variants differing from the parent polypeptide by one amino acid, with each amino acid substitution being correlated to structural/functional attributes at any position in the polypeptide. Saturation mutagenesis uses PCR and primers homologous to the parent sequence wherein one or more codon encoding nucleotide triplets is randomized. Randomization results in the incorporation of codons corresponding to all amino acid replacements in the final, translated polypeptide. Each PCR product is genetically engineered into an expression vector to be introduced into an expression host and screened for structural and functional attributes through clonal screening assays and other methods as described herein.

In one aspect of saturation mutagenesis, correlated saturation mutagenesis (“CSM”) is used wherein two or more amino acids at rationally designated positions are changed concomitantly to different amino acid residues to engineer improved enzyme function and structure. Correlated saturation mutagenesis allows for the identification of complimentary amino acid changes having, e.g., positive, synergistic effects on Aoa enzyme structure and function. Such synergistic effects include, but are not limited to, significantly altered enzyme stability, substrate affinity, substrate specificity or catalytic turnover rate, independently or concomitantly increasing advantageously the production of 1-alkenes.

In yet another embodiment, amino acid substitution combinations of CSM derived protein variants being optimized for a particular function are combined with one or more CSM derived protein variants being optimized for another particular function to derive a 1-alkene synthase, alpha-olefin-associated enzyme and/or a phosphopantetheinyl transferase variant exhibiting multiple optimized structural and functional characteristics. For example, amino acid changes in combinatorial mutants showing optimized protomer interactions are combined with amino acid changes in combinatorial mutants showing optimized catalytic turnover.

In one embodiment, mutational variants derived from the methods described herein are cloned. DNA sequences produced by saturation mutagenesis are designed to have restriction sites at the ends of the gene sequences to allow for excision and transformation into a host cell plasmid. Generated plasmid stocks are transformed into a host cell and incubated at optimal growth conditions to identify successfully transformed colonies.

Another embodiment utilizes gene shuffling (P. Stemmer, Nature (1994) Vol. 370:389-391) or gene reassembly (U.S. Pat. No. 5,958,672) to develop improved protein structure/function through the generation of chimeric proteins. With gene shuffling, two or more homologous Aoa enzyme encoding nucleotide sequences are treated with endonucleases at random positions, mixed together, heated until sufficiently melted and reannealed. Nucleotide sequences from homologues will anneal to develop a population of chimeric genes that are repaired to fill in any gaps resulting from the re-annealing process, expressed and screened for improved structure/function alpha-olefin-associated enzyme or 1-alkene synthase chimeras. Gene reassembly is similar to gene shuffling; however, nucleotide sequences for specific, homologous alpha-olefin-associated enzyme or 1-alkene synthase protein domains are targeted and swapped with other homologous domains for reassembly into a chimeric gene. The genes are expressed and screened for improved structure/function alpha-olefin-associated enzyme or 1-alkene synthase chimeras.

In a further embodiment any and/or all sequences additionally are expression optimized for the specific expression host cell.

### Methods for Measuring Protein Variant Efficacy

Variations in expressed polypeptide sequences may result in measurable differences in the whole-cell rate of substrate conversion. It is desirable to determine differences in the rate of substrate conversion by assessing productivity in a host cell having a particular protein variant relative to other whole cells having a different protein variant. Additionally, it would be desirable to determine the efficacies of whole-cell substrate conversion as a function of environmental factors including, but not limited to, pH, temperature nutrient concentration and salinity.

Therefore, in one embodiment, the biophysical analyses described herein on protein variants are performed to measure structural/functional attributes. Standard analyses of polypeptide activity are well known to one of ordinary skill in the art. Such analysis can require the expression and high purification of large quantities of polypeptide, followed by various physical methods (including, but not limited to, calorimetry, fluorescence, spectrophotometric, spectrometric, liquid chromatography (LC), mass spectrometry (MS), LC-MS, affinity chromatography, light scattering, nuclear magnetic resonance and the like) to assay function in a specific environment or functional differences among homologues.

In another embodiment, the polypeptides are expressed, purified and subject to the aforementioned analytical techniques to assess the functional difference among polypeptide sequence homologues, for example, the rate of substrate conversion and/or 1-alkene synthesis.

Batch culture (or closed system culture) analysis is well known in the art and can provide information on host cell population effects for host cells expressing genetically engineered genes. In batch cultures a host cell population will grow until available nutrients are depleted from the culture media.

In one embodiment, the polypeptides are expressed in a batch culture and analyzed for approximate doubling times, expression efficacy of the engineered polypeptide and end-point net product formation and net biomass production.

Turbidostats are well known in the art as one form of a continuous culture within which media and nutrients are provided on an uninterrupted basis and allow for non-stop propagation of host cell populations. Turbidostats allow the user to determine information on whole cell propagation and steady-state productivity for a particular biologically produced end product such as host cell doubling time, temporally delimited biomass production rates for a particular host cell population density, temporally delimited host cell population density effects on substrate conversion and net productivity of a host cell substrate conversion. Turbidostats can be designed to monitor the partitioning of substrate conversion products to the liquid or gaseous state. Additionally, quantitative evaluation of net productivity of a carbon-based product of interest can be accurately performed due to the exacting level of control that one skilled in the art has over the operation of the turbidostat. These types of information are useful to assess the parsed and net efficacies of a host cell genetically engineered to produce a specific carbon-based product of interest.

In one embodiment, identical host cell lines differing only in the nucleic acid and expressed polypeptide sequence of a homologous enzyme are cultured in a uniform-environment turbidostat to determine highest whole cell efficacy for the desired carbon-based product of interest.

In another embodiment, identical host cell lines differing only in the nucleic acid and expressed polypeptide sequence of a homologous enzyme are cultured in a batch culture or a turbidostat in varying environments (e.g. temperature, pH, salinity, nutrient exposure) to determine highest whole cell efficacy for the desired carbon-based product of interest.

In one embodiment, mutational variants derived from the methods described herein are cloned. DNA sequences produced by saturation mutagenesis are designed to have restriction sites at the ends of the gene sequences to allow for cleavage and transformation into a host cell plasmid. Generated plasmid stocks are transformed into a host cell and incubated at optimal growth conditions to identify successfully transformed colonies.

### Methods for Producing 1-Nonadecene

It is desirable to engineer into an organism better suited for industrial use a genetic system from which 1-nonadecene can be produced efficiently and cleanly.

Accordingly, an embodiment of the invention includes the conversion of water, an inorganic carbon source (e.g., carbon dioxide), and light into 1-alkenes using the alpha-olefin-associated enzyme and/or 1-alkene synthase enzyme described herein. In one embodiment, the invention includes producing 1-alkenes, including 1-heptadecene, 1-nonadecene, 1-octadecene, and 1,x-nonadecadiene using genetically engineered host cells expressing an alpha-olefin-associated enzyme and/or 1-alkene synthase gene. In one aspect, the alpha-olefin-associated enzyme, 1-alkene synthase, or protein in a 1-alkene synthase pathway is engineered to interact with a substrate of a selected chain length. In another aspect, the alpha-olefin-associated enzyme, 1-alkene synthase, or protein in a 1-alkene synthase pathway is engineered to alter the length of alpha-olefins produced in a cell containing the engineered protein(s).

In another preferred embodiment, the genetically engineered host cells expresses an alpha-olefin-associated enzyme and one or more genes in a 1-alkene biosynthetic pathway enabling the host cell to convert water, light, and an inorganic carbon source (e.g., carbon dioxide and/or stearic acid) into 1-nonadecene.

In another embodiment of the invention, the genetically engineered host cell is processed into an enzymatic lysate for performing the above conversion reaction. In yet another embodiment, the aoa gene product is purified, as described herein, for carrying out the conversion reaction.

The host cells and/or enzymes, for example in the lysate, partially purified, or purified, used in the conversion reactions are in a form allowing them to perform their intended function, producing a desired compound, for example, 1-nonadecene. The microorganisms used can be whole cells, or can be only those portions of the cells necessary to obtain the desired end result. The microorganisms can be suspended (e.g., in an appropriate solution such as buffered solutions or media), rinsed (e.g., rinsed free of media from culturing the microorganism), acetone-dried, immobilized (e.g., with polyacrylamide gel or k-carrageenan or on synthetic supports, for example, beads, matrices and the like), fixed, cross-linked or permeabilized (e.g., have permeabilized membranes and/or walls such that compounds, for example, substrates, intermediates or products can more easily pass through said membrane or wall).

In yet another embodiment, a purified or unpurified alpha-olefin-associated enzyme and/or 1-alkene synthesizing enzyme (e.g., a 1-alkene synthase) is used in the conversion reactions. The enzyme is in a form that allows it to perform its intended function. For example, the enzyme can be immobilized, conjugated or floating freely.

In yet another embodiment the alpha-olefin-associated enzymes and/or 1-alkene synthase enzymes are chimeric wherein a polypeptide linker is encoded between the above enzyme and another enzyme. Upon translation into a polypeptide, two enzymes are tethered together by a polypeptide linker. Such arrangement of two or more functionally related proteins tethered together in a host cell increases the local effective concentration of metabolically related enzymes that can increase the efficiency of substrate conversion. In one embodiment, an alpha-olefin-associated enzyme and 1-alkene synthase enzyme are linked by a polypeptide linker.

The following examples are for illustrative purposes and are not intended to limit the scope of the invention.

### Example 1

### Improved Yields of 1-Alkenes by Co-Expression of Aoa with NonA in Escherichia coli

### Strain Construction

The Synechococcus sp. PCC 7002 nonA (Genbank NC_{—}010475, locus A1173) was purchased from DNA 2.0 following codon optimization, checking for mRNA secondary structure effects, removal of unwanted restriction sites, insertion of unique restriction sites flanking domains and appending N- and C-terminal Strep-tag II and His tags. The gene and encoded protein sequence for this optimized gene (nonA_optV6) is given in SEQ ID NO:2 and SEQ ID NO:3, respectively. The broad spectrum phosphopantetheinyl transferase sfp (Quadri et al. 1998, Genbank protein P39135.2) was purchased from DNA 2.0 following codon optimization, checking for mRNA secondary structure effects and removal of unwanted restriction sites (SEQ ID NO:1). The Synechococcus sp. PCC 7002 aoa (Genbank NC_{—}010475, locus A2265) was amplified from Synechococcus sp. PCC 7002 genomic DNA using the PCR primers A2265 FP SacI (ggGAGCTCaaggaattatagttatgcgcaaaccctggttaga (SEQ ID NO: 24)) and A2265 RP SbfI (ggCCTGCAGGttatagggactggatcgccagttttttctgct (SEQ ID NO: 25)) and the Phusion high-fidelity PCR kit (New England Biolabs) following the manufacturer's instructions. NonA_optV6 was cloned into the NdeI-MfeI and sfp was cloned into the NcoI-EcoRI restriction sites of pCDFDuet-1 (Novagen) to yield pJB1412. The aoa gene was cloned into the SacI-SbfI restriction sites of pJB1412 to yield pJB1522. These two plasmids and pCDFDuet-1 were transformed into chemically competent E. coli BL21 DE(3) (Invitrogen) following the manufacturer's directions (Table 2).

TABLE 2
Joule Culture Collection (JCC) numbers of the BL21 DE(3)
strains investigated for the production of 1-alkenes

| Strain | Plasmid | Genes |
|---|---|---|
| JCC308 | pCDFDuet-1 | — |
| JCC2094 | pJB1412 | sfp, nonA_optV6 |
| JCC2157 | pJB1523 | sfp, nonA_optV6, aoa |

### Culture Conditions and Sampling

Single colonies of JCC308, JCC2094 and JCC2157 from LB plates containing 1% glucose and 50 mg/L spectinomycin were grown for 6 h at 37° C. in 4 ml of LB medium containing the same glucose and antibiotic concentration. These starter cultures were used to inoculate 15 ml cultures at a starting OD600 of 0.05 in a 2% casamino acid M9-derived medium that was amended to increase M9 concentration of phosphate by three-fold (33.9 g/L Na2HPO4 and 9 g/L KH2PO4) and was supplemented with 3 mg/L FeSO4.7H2O and 0.01 mM IPTG. The cultures were incubated for 68 h at 30° C. at 225 rpm in a New Brunswick shaking incubator. 50 μl of the cultures were removed to determine the OD600 and the remaining volume of the cultures (13 ml) was pelleted by centrifugation. The supernatant was discarded, the cells resuspended in 1 ml of milli-Q water, transferred to a microcentrifuge tube and pelleted by centrifugation. After removing residual aqueous medium, the cell pellets were vortexed for 20 seconds in 1 ml of acetone (Acros Organics 326570010) containing 25 mg/L butylated hydroxytoluene (antioxidant) and 25 mg/L eicosane (internal standard). The debris was pelleted by centrifugation and the acetone supernatants were analyzed for the presence of 1-alkenes.

### Identification and Quantification of 1-Alkenes

An Agilent 7890A GC/5975C ELMS equipped with a 7683B autosampler was used to identify the 1-alkenes. One μL of each sample was injected into the GC inlet using pulsed splitless injection (pressure: 20 psi, pulse time: 0.3 min, purge time: 0.2 min, purge flow: 15 mL/min) and an inlet temperature of 290° C. The column was a HP-5MS-UI (Agilent, 20 m×0.18 mm×0.18 μm) and the carrier gas was helium at a flow of 0.72 mL/min. The GC oven temperature program was 80° C., hold 0.3 minute; 17.6°/min increase to 290° C.; hold six minutes. The GC/MS interface was 290° C., the MS mass range monitored was 25 to 400 amu and the temperatures of the source and quadrupole were 230° C. and 150° C., respectively. 1-nonadecene (rt 8.4 min), 1-octadecene (rt 7.8 min) and 1-heptadecene (rt 7.2 min) were identified based on comparison of their mass spectra (NIST MS database; 2008) and retention times with authentic standards. The C19:2 1,x-nonadecadiene (rt 8.3) was identified based on interpretation of the mass spectrum and a chemically consistent retention time.

An Agilent 7890A GC/FID equipped with a 7683 series autosampler was used to quantify the 1-alkenes. One μL of each sample was injected into the GC inlet (split 8:1, pressure: 20 psi, pulse time: 0.3 min, purge time: 0.2 min, purge flow: 15 mL/min) which had an inlet temperature of 290° C. The column was a HP-5MS (Agilent, 20 m×0.18 mm×0.18 μm) and the carrier gas was helium at a flow of 1.0 mL/min. The GC oven temperature program was 80° C., hold 0.3 minute; 17.6°/min increase to 290° C.; hold 6 minutes. Calibration curves were constructed for the 1-alkenes (1-nonadecene, 1-octadecene and 1-heptadecene) using commercially available standards (Sigma-Aldrich), and the concentrations of the 1-alkenes present in the extracts were determined based on the linear regressions of the peak areas and concentrations. The concentration of 1-nonadecadiene in the samples was determined using the calibration curve for 1-nonadecene. The concentrations of the compounds were normalized to the internal standard (eicosane) and reported as mg/L of culture.

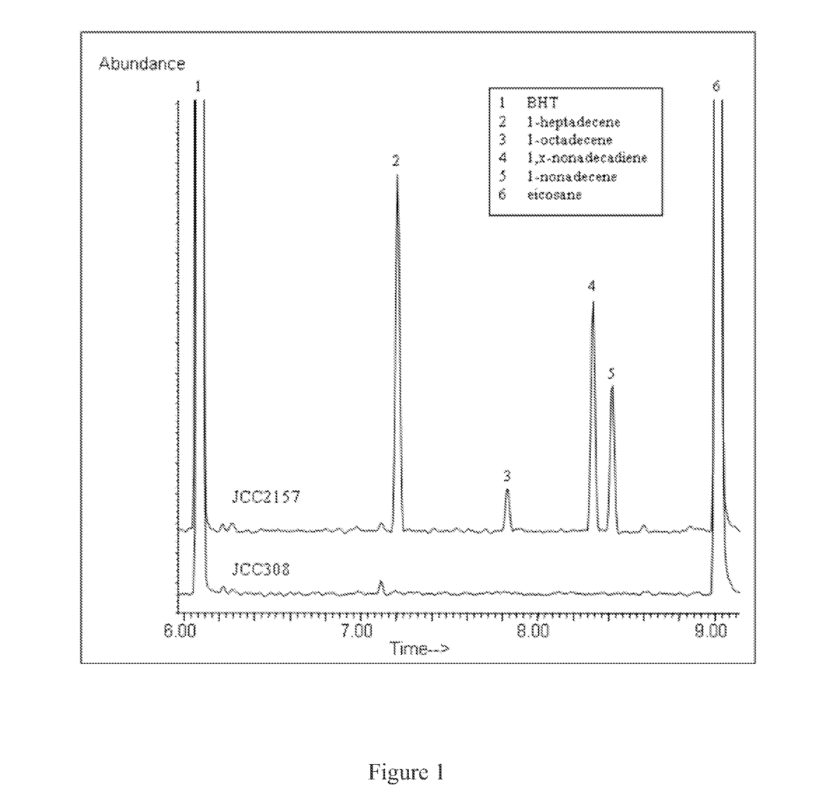

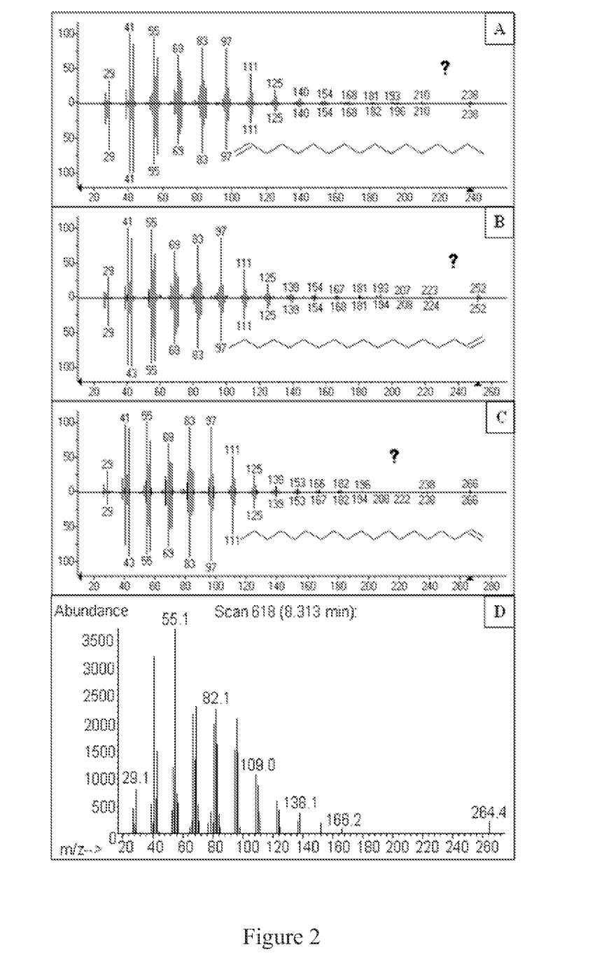

The total ion count (TIC) chromatograms for JCC2157 and JCC308 are shown in FIG. 1. Four 1-alkenes are present in JCC2157 that are not found in JCC308. The mass spectra for the 1-alkenes and comparison with authentic standards where possible are shown in FIG. 2. The quantification data from the experiment is summarized in Table 3. The strain bearing aoa (JCC2157) produced greater than four times the amount of 1-alkenes than the strain only expressing nonA_optV6 and sfp (i.e., not expressing aoa).

TABLE 3
The optical densities of the cultures and the total
mg/L of 1-alkenes produced by the BL21 DE(3) strains.
The % DCW was estimated based on the OD measurement
using an average of 400 mg L-1 OD600-1.

| | | | 1-alkenes |
|---|---|---|---|
| | | 1-alkenes | (% of |
| Strain | OD₆₀₀ | (mg/L) | DCW) |
| JCC308 | 2.7 | — | — |
| JCC2094 | 2.9 | 0.06 | 0.005 |
| JCC2157 | 3.2 | 0.28 | 0.022 |

### Example 2

### Improved and Regulated Expression of 1-Alkenes in Synechococcus Sp. PCC 7002

### Strain Construction

The Synechococcus sp. PCC 7002 nonA (Genbank NC_{—}010475, locus A1173) was purchased from DNA 2.0 following codon optimization, checking for mRNA secondary structure effects, removal of unwanted restriction sites, insertion of unique restriction sites flanking domains and appending N- and C-terminal Strep-tag II and His tags. The gene and encoded protein sequence for this optimized gene (nonA_optV6) is given in SEQ ID NO: 2 and 3, respectively. The Synechococcus sp. PCC 7002 aoa (Genbank NC_{—}010475, locus A2265) was amplified from Synechococcus sp. PCC 7002 genomic DNA using the Phusion high-fidelity PCR kit (New England Biolabs) following the manufacturer's instructions and was modified to contain a C-terminal Strep-tag II and His tag (SEQ ID NO:18 (nucleotide) and SEQ ID NO: 19 (protein)) to produce aoaH6SII. These genes were cloned in a divergent manner such that the expression of aoaH6SII was controlled by a moderate strength constitutive tsr2142 promoter (SEQ ID NO: 20) and nonA_optV6 was controlled by a urea-repressible ompR promoter (SEQ ID NO: 21). This divergent operon was assembled in a SYNPCC7002A_{—}0358 targeting vector containing 750 bp of upstream and downstream homology designed to allow insertion of the nonA_optV6 and tagged aoa expression cassette into the chromosome. An aadA gene (SEQ ID NO: 22) is present as well to allow selection of colonies containing the genes with spectinomycin. The sequence and annotation of this plasmid (pJB2580) is provided in SEQ ID 23. This plasmid was naturally transformed into JCC1218 (as described in PCT/US2010/0330642, hereby incorporated by reference in its entirety) using a standard cyanobacterial transformation and segregation protocol yielding JCC4124. The genotypes of the three strains of cyanobacteria are provided in Table 4.

TABLE 4
Joule Culture Collection (JCC) numbers of the Synechococcus sp. PCC
7002-based strains investigated for the production of 1-alkenes.

| Strain | Genotype |
|---|---|
| JCC138 | Synechococcus sp. PCC 7002 |
| JCC1218 | JCC138 ΔnonA |
| JCC4124 | JCC1218 A0358::P(tsr2142)-aoaH6SII-P(ompR)- |
| | nonA_optV6 |

Culture Conditions and Sampling:

A clonal culture of three strains described in Table 4 was grown in A+ medium supplemented with 15 mM urea and the appropriate antibiotics for the respective strains (JCC138: no antibiotic, JCC1218: 50 mg/L gentamycin, JCC4124: 50 mg/L gentamycin and 100 mg/L spectinomycin). The strains were incubated for five days at 30° C. at 150 rpm in 3% CO₂-enriched air at ˜100 μmol photons m⁻² s⁻¹ in a Multitron II (Infors) shaking photoincubator. These cultures were then used to inoculate duplicate 30 ml cultures of JB2.1 (as described in PCT/US2009/006516, hereby incorporated by reference in its entirety) containing either 2 mM or 15 mM urea, resulting in four flasks per strain. JB2.1 medium consists of 18.0 g/l sodium chloride, 5.0 g/l magnesium sulfate heptahydrate, 4.0 g/l sodium nitrate, 1.0 g/l Tris, 0.6 g/l potassium chloride, 0.3 g/l calcium chloride (anhydrous), 0.2 g/l potassium phosphate monobasic, 34.3 mg/l boric acid, 29.4 mg/l EDTA (disodium salt dihydrate), 14.1 mg/l iron (III) citrate hydrate, 4.3 mg/l manganese chloride tetrahydrate, 315.0 μg/l zinc chloride, 30.0 μg/l molybdenum (VI) oxide, 12.2 μg/l cobalt (II) chloride hexahydrate, 10.0 μg/l vitamin B12, and 3.0 μg/l copper (II) sulfate pentahydrate. The 12 cultures were grown for 7 days at 37° C. at 150 rpm in 3% CO₂-enriched air at −100 μmol photons m⁻² s⁻¹ in a Multitron II (Infors) shaking photoincubator. The cultures were sampled six times over three days and once on day 7 after addition of water at each timepoint to compensate for loss of water due to evaporation. Cultures were monitored for growth by taking OD730 measurements and either 500 μl of culture (first three timepoints) or 250 μl of culture (remaining timepoints) for 1-alkene extraction. The samples were transferred to a microcentrifuge tube and pelleted by centrifugation and the aqueous supernatant was discarded. After centrifuging the pellets once more and removing any residual aqueous medium, the cell pellets were vortexed for 20 seconds in 500 μl of acetone (Acros Organics 326570010) containing 25 mg/L butylated hydroxytoluene (antioxidant) and 25 mg/L eicosane (internal standard). The debris was pelleted by centrifugation and the acetone supernatants were analyzed for the presence of 1-alkenes.

### Identification and Quantification of 1-Alkenes

An Agilent 7890A GC/FID equipped with a 7683 series autosampler was used to quantify the 1-alkenes. One μL of each sample was injected into the GC inlet (split ratio 50:1) which had an inlet temperature of 290° C. The column was a Rxi-5MS (Restek, 10 m×0.10 mm×0.1 μm) and the carrier gas was helium at a flow of 1.5 mL/min. The GC oven temperature program was 90° C., hold 0.5 minute; 30° C./min increase to 290° C.; total run time 10.17 min). Calibration curves were constructed for a panel of 1-alkenes (1-nonadecene, 1-octadecene, 1-heptadecene, 1-hexadecene, 1-pentadecene, 1-tetradecene and 1-tridecene) using commercially available standards (Sigma-Aldrich), and the concentration of the 1-nonadecene present in the extracts was determined based on the linear regressions of the peak area and concentration. The concentration of 1-nonadecene was normalized to the internal standard (eicosane) and reported as mg/L of culture.

The GC/FID chromatograms for the JCC138, JCC1218 and JCC4124 cultures incubated in 2 mM urea at day 7 are shown in FIG. 1. JCC138 and JCC4124 both produced 1-nonadecene while JCC1218 did not. The 1-nonadecene production and growth of the cultures is shown in FIG. 2 and the 1-nonadecene production rate of the three strains during the first four timepoints is given in Table 5. JCC4124 has >6× higher 1-nonadecene production rate in 2 mM urea than JCC138 but demonstrates comparable production when incubated in 15 mM urea showing that the pathway is attenuated in the high urea condition. After day 3, 1-nonadecene production is induced in the JCC4124 15 mM urea cultures since the reduced nitrogen is consumed (FIG. 2).

TABLE 5
The 1-nonadecene production rate of the three strains in 2 mM urea
(U2) or 15 mM urea (U15) over the first four timepoints (through
day 2). The rates were determined from the averaged 1-nonadecene
data from the duplicate flasks for each strain and condition.

| | 1-nonadecene |
|---|---|
| | production rate |
| Strain | (mg L⁻¹ h⁻¹) |
| JCC1218 U2 | 0 |
| JCC1218 U15 | 0 |
| JCC138 U2 | 0.031 |
| JCC138 U15 | 0.034 |
| JCC4124 U2 | 0.190 |
| JCC4124 U15 | 0.022 |

Complete citations to various articles referred to herein are provided below:
Gu, L., Wang, B., Kulkarni, A., Gehret, J. J., Lloyd, K. R., Gerwick, L., Gerwick, W. H., Wipf, P., Håkannson, K., Smith, J. L. and Sherman, D. H. 2009. Polyketide decarboxylative chain termination preceded by O-sulfonation in curacin A biosynthesis. Journal of the American Chemical Society 131: 16033-16035.
Mendez-Perez, D., Begemann, M. B. and Pfleger, B. F. 2011. Modular synthase-encoding gene involved in α-olefin biosynthesis in Synechococcus sp. strain PCC 7002. Applied and Environmental Microbiology 77: 4264-4267.
Quadri, L. E. N., Weinreb, P. H., Ming, L., Nakano, M. M., Zuber, P. and Walsh, C. T. 1998. Characterization of Sfp, a Bacillus subtilis phosphopantetheinyl transferase for peptidyl carrier protein domains in peptide synthetases. Biochemistry 37: 1585-1595.

All publications, patents and other references mentioned herein are hereby incorporated by reference in their entireties and for all purposes.

INFORMAL SEQUENCE LISTING

SEQ ID NO: 1
sfp (codon optimized)
ATGAAAATTTACGGCATTTACATGGACCGTCCTTTGAGCCAAGAAGAAAATGAGCGTTTTATGTCGTT
CATCAGCCCGGAAAAACGCGAGAAGTGCCGTCGTTTCTATCATAAGGAGGATGCCCATCGCACGCTGC
TGGGTGATGTTCTGGTTCGTTCCGTGATCTCCCGCCAATACCAGCTGGACAAAAGCGATATCCGCTTT
TCCACCCAGGAGTACGGCAAACCATGTATCCCGGACCTGCCGGACGCTCACTTCAACATTAGCCACAG
CGGTCGTTGGGTGATTTGTGCGTTCGATAGCCAGCCGATTGGTATTGACATTGAAAAGACGAAGCCTA
TTAGCCTGGAGATCGCCAAGCGCTTCTTCAGCAAAACCGAGTATAGCGATCTGCTGGCGAAAGACAAA
GACGAGCAAACCGACTACTTTTACCACCTGTGGAGCATGAAAGAAAGCTTTATCAAGCAAGAAGGTAA
GGGTTTGAGCTTGCCGCTGGACAGCTTTAGCGTGCGTCTGCATCAGGATGGTCAGGTCAGCATCGAGC
TGCCGGACTCTCACTCTCCGTGCTATATTAAAACCTACGAGGTCGATCCGGGCTATAAAATGGCGGTT
TGCGCAGCACACCCGGACTTTCCGGAGGATATCACTATGGTGAGCTATGAAGAGTTGCTGTAA
SEQ ID NO: 2
nonA_optV6 (nucleotide sequence)
ATGGCAAGCTGGTCCCACCCGCAATTCGAGAAAGAAGTACATCACCATCACCATCATGGCGCAGTGGG
CCAGTTTGCGAACTTTGTAGACCTGTTGCAATACCGTGCCAAGCTGCAAGCACGTAAGACCGTCTTTA
GCTTCCTGGCGGACGGCGAAGCGGAGAGCGCCGCTCTGACCTATGGTGAGCTGGATCAAAAGGCGCAG
GCAATCGCGGCGTTCCTGCAAGCAAATCAGGCACAAGGCCAACGTGCATTGCTGCTGTATCCGCCAGG
TCTGGAGTTCATCGGTGCCTTCCTGGGTTGTCTGTATGCGGGTGTCGTCGCGGTTCCGGCATATCCTC
CGCGTCCGAACAAGTCCTTCGACCGTTTGCACTCCATCATTCAGGACGCCCAAGCGAAGTTTGCACTG
ACGACGACCGAGTTGAAGGATAAGATTGCAGACCGTCTGGAAGCGCTGGAGGGTACGGACTTCCATTG
CCTGGCGACCGACCAAGTCGAGCTGATCAGCGGCAAAAACTGGCAAAAGCCGAATATCTCCGGTACGG
ATCTGGCGTTTCTGCAATACACCAGCGGCAGCACGGGTGATCCAAAAGGCGTGATGGTCAGCCACCAT
AACCTGATTCACAATAGCGGTCTGATTAACCAGGGTTTCCAAGACACCGAAGCGAGCATGGGTGTGTC
CTGGCTGCCGCCGTATCACGACATGGGTCTGATTGGCGGCATCCTGCAACCTATCTACGTTGGCGCAA
CGCAAATCCTGATGCCACCAGTCGCCTTTCTGCAACGTCCGTTCCGCTGGCTGAAGGCGATCAACGAT
TACCGTGTCAGCACCAGCGGTGCGCCGAACTTTGCTTACGACCTGTGCGCTTCTCAGATTACCCCGGA
ACAAATCCGCGAGCTGGATCTGAGCTGTTGGCGTCTGGCATTCAGCGGTGCAGAGCCGATTCGCGCTG
TCACGCTGGAAAACTTTGCGAAAACGTTCGCAACCGCGGGTTTCCAGAAATCGGCCTTCTACCCTTGT
TACGGTATGGCGGAAACCACCCTGATCGTGAGCGGTGGCAATGGCCGTGCCCAACTGCCACAGGAGAT
CATCGTTAGCAAGCAGGGCATTGAGGCGAACCAAGTGCGTCCGGCTCAAGGCACGGAAACGACCGTGA
CCCTGGTGGGTAGCGGTGAGGTCATTGGTGACCAGATCGTTAAGATCGTTGACCCTCAAGCGCTGACC
GAGTGCACCGTCGGTGAAATTGGCGAGGTGTGGGTTAAAGGTGAAAGCGTTGCTCAGGGCTACTGGCA
GAAGCCGGACTTGACGCAGCAGCAGTTCCAGGGTAACGTGGGTGCCGAAACGGGTTTCCTGCGCACCG
GCGATCTGGGTTTCCTGCAAGGCGGCGAGCTGTATATCACCGGCCGTCTGAAGGATCTGCTGATCATT
CGTGGCCGTAATCACTATCCTCAGGACATTGAGCTGACCGTGGAAGTTGCTCACCCAGCCCTGCGTCA
GGGCGCAGGTGCCGCGGTGAGCGTGGACGTTAATGGTGAAGAACAACTGGTGATCGTTCAAGAGGTTG
AGCGTAAGTACGCACGCAAGCTGAATGTGGCAGCAGTCGCTCAGGCCATCCGTGGTGCGATTGCGGCA
GAGCACCAGTTGCAGCCGCAGGCGATCTGCTTTATCAAACCGGGCAGCATCCCGAAAACTAGCAGCGG
CAAAATCCGTCGTCACGCATGTAAGGCCGGTTTTCTGGACGGAAGCTTGGCGGTTGTTGGTGAGTGGC
AACCGAGCCATCAGAAAGAGGGCAAAGGTATTGGTACCCAGGCAGTGACCCCGAGCACCACGACGTCC
ACCAACTTTCCGCTGCCGGATCAACACCAGCAACAGATCGAGGCGTGGCTGAAGGACAACATCGCGCA
CCGCCTGGGTATTACGCCGCAGCAGTTGGATGAAACGGAACCGTTCGCTTCTTACGGTCTGGACAGCG
TTCAAGCAGTCCAGGTCACCGCAGACCTGGAGGACTGGCTGGGCCGCAAGCTGGACCCGACTCTGGCC
TATGATTACCCGACCATTCGCACGCTGGCGCAATTCCTGGTTCAGGGCAACCAGGCCTTGGAGAAAAT
CCCGCAAGTTCCAAAGATTCAGGGTAAAGAGATTGCGGTGGTGGGCCTGAGCTGCCGCTTTCCGCAGG
CGGACAATCCGGAGGCGTTCTGGGAACTGTTGCGCAATGGCAAGGATGGCGTGCGTCCGCTGAAAACC
CGTTGGGCCACTGGTGAGTGGGGTGGTTTCCTGGAGGATATCGACCAGTTTGAGCCGCAGTTCTTTGG
TATTAGCCCGCGTGAGGCGGAGCAAATGGACCCGCAACAGCGTCTGCTGCTGGAGGTCACCTGGGAGG
CACTGGAGCGTGCGAATATCCCTGCCGAATCCCTGCGTCACAGCCAGACCGGCGTCTTTGTGGGCATT
AGCAACAGCGATTACGCACAACTGCAAGTGCGTGAGAACAACCCGATCAATCCGTACATGGGTACTGG
TAACGCACATAGCATCGCGGCGAATCGTCTGAGCTACTTTCTGGATCTGCGCGGTGTCTCCCTGAGCA
TTGATACCGCGTGTTCTAGCAGCCTGGTCGCAGTTCATCTGGCGTGCCAAAGCCTGATTAACGGCGAG
AGCGAGCTGGCGATTGCTGCGGGTGTTAATCTGATTCTGACCCCGGATGTCACGCAAACCTTTACCCA
AGCGGGTATGATGAGCAAGACGGGCCGTTGCCAGACGTTTGATGCGGAGGCGGACGGCTACGTGCGCG
GTGAAGGCTGCGGCGTTGTTCTGCTGAAACCGCTGGCTCAGGCGGAGCGTGATGGCGACAATATCCTG
GCGGTCATCCACGGTAGCGCGGTTAACCAGGACGGTCGCAGCAATGGTCTGACTGCGCCGAACGGCCG
CTCTCAGCAAGCGGTTATCCGTCAGGCCCTGGCGCAGGCGGGCATCACCGCGGCAGACCTGGCGTATT
TGGAAGCGCATGGTACGGGCACCCCGCTGGGCGACCCGATTGAAATCAACAGCTTGAAAGCAGTGCTG
CAAACCGCCCAGCGCGAGCAACCGTGCGTTGTGGGCAGCGTCAAGACGAACATTGGCCACCTGGAGGC
AGCAGCGGGTATTGCAGGTCTGATCAAGGTGATTCTGTCCCTGGAGCACGGCATGATTCCGCAACACC
TGCACTTTAAGCAACTGAATCCGCGCATCGACCTGGACGGCCTGGTTACCATCGCGAGCAAAGACCAG
CCGTGGTCGGGTGGTAGCCAGAAGCGTTTCGCCGGTGTCAGCAGCTTTGGTTTTGGCGGTACGAATGC
TCACGTGATTGTTGGTGATTATGCCCAGCAAAAGTCCCCGCTGGCTCCGCCTGCGACCCAAGACCGTC
CTTGGCATCTGCTGACTCTGAGCGCGAAGAACGCACAAGCGTTGAACGCGTTGCAAAAGAGCTATGGT
GACTACCTGGCGCAACATCCGAGCGTTGACCCTCGCGATCTGTGCCTGAGCGCTAACACTGGTCGCTC
TCCGCTGAAAGAACGCCGCTTCTTCGTGTTCAAGCAGGTTGCCGACTTGCAACAAACCCTGAATCAGG
ACTTTCTGGCGCAGCCGAGGCTGAGCAGCCCAGCCAAGATTGCGTTCCTGTTCACGGGTCAGGGCAGC
CAGTACTACGGTATGGGCCAGCAACTGTATCAGACGTCCCCGGTTTTCCGTCAAGTCCTGGATGAATG
CGACCGTCTGTGGCAGACGTACAGCCCGGAGGCACCGGCGCTGACCGATCTGCTGTACGGCAATCATA
ATCCTGACCTGGTTCATGAAACGGTTTACACGCAACCGCTGCTGTTCGCGGTGGAGTATGCTATCGCG
CAGTTGTGGTTGAGCTGGGGCGTTACTCCGGATTTCTGCATGGGTCATAGCGTCGGTGAGTATGTGGC
GGCCTGCCTGGCGGGTGTGTTTAGCCTGGCGGATGGCATGAAACTGATTACCGCGCGTGGTAAACTGA
TGCATGCACTGCCGAGCAATGGCAGCATGGCGGCTGTGTTTGCGGACAAAACCGTTATCAAGCCGTAT
CTGAGCGAACACCTGACCGTCGGCGCAGAAAATGGCAGCCACCTGGTTCTGAGCGGTAAGACCCCTTG
TCTGGAAGCATCCATCCACAAACTGCAAAGCCAGGGCATCAAAACCAAGCCTCTGAAAGTCTCCCATG
CGTTCCACTCGCCGCTGATGGCGCCGATGCTGGCGGAATTTCGTGAGATCGCCGAACAGATTACGTTC
CATCCGCCACGTATCCCGCTGATTAGCAACGTGACGGGTGGTCAAATCGAGGCCGAGATCGCGCAAGC
AGACTATTGGGTTAAACATGTTAGCCAGCCGGTGAAGTTCGTTCAGAGCATTCAGACCCTGGCCCAAG
CGGGTGTGAATGTGTACCTGGAAATCGGTGTTAAACCAGTCCTGCTGTCTATGGGTCGCCACTGTCTG
GCAGAGCAGGAAGCGGTTTGGCTGCCGAGCCTGCGTCCACATAGCGAGCCTTGGCCGGAAATCTTGAC
TAGTCTGGGCAAACTGTACGAGCAAGGTCTGAATATCGACTGGCAAACGGTTGAAGCCGGTGATCGCC
GTCGTAAGCTGATTTTGCCGACCTACCCGTTCCAGCGTCAGCGTTATTGGTTCAACCAAGGTAGCTGG
CAAACCGTCGAAACTGAGAGCGTGAATCCAGGCCCGGACGACCTGAATGACTGGCTGTACCAAGTGGC
ATGGACTCCGCTGGATACGCTGCCGCCTGCACCGGAACCGTCGGCGAAACTGTGGCTGATTCTGGGTG
ATCGTCACGATCACCAACCGATTGAGGCCCAGTTCAAAAACGCCCAACGTGTGTACCTGGGCCAAAGC
AACCACTTTCCGACGAACGCCCCGTGGGAGGTGAGCGCGGACGCACTGGATAACTTGTTTACCCATGT
GGGTAGCCAAAACCTGGCAGGCATTCTGTATCTGTGCCCGCCTGGTGAAGATCCGGAGGATCTGGATG
AGATTCAGAAACAAACTTCCGGCTTTGCGTTGCAACTGATTCAGACCCTGTATCAGCAGAAAATCGCA
GTGCCGTGTTGGTTTGTTACCCATCAAAGCCAGCGTGTGCTGGAAACGGACGCGGTGACGGGTTTTGC
CCAAGGTGGTCTGTGGGGTTTGGCGCAAGCGATTGCACTGGAACATCCGGAACTGTGGGGTGGTATCA
TTGACGTGGATGATAGCCTGCCGAACTTCGCGCAGATTTGTCAGCAACGTCAGGTTCAGCAACTGGCT
GTCCGTCACCAGAAACTGTATGGTGCGCAACTGAAGAAGCAGCCGAGCCTGCCGCAGAAGAATCTGCA
GATCCAACCTCAACAGACCTACCTGGTCACGGGCGGTTTGGGTGCAATCGGTCGTAAGATTGCGCAGT
GGCTGGCGGCTGCGGGTGCTGAGAAAGTTATCCTGGTTAGCCGTCGTGCACCGGCAGCGGATCAACAA
ACCTTGCCGACCAACGCCGTGGTGTACCCGTGCGATCTGGCGGATGCGGCGCAGGTTGCGAAACTGTT
CCAAACCTATCCGCACATTAAGGGTATCTTTCATGCAGCCGGTACGCTGGCTGACGGTTTGCTGCAAC
AGCAAACCTGGCAGAAATTCCAGACTGTCGCTGCGGCGAAGATGAAGGGCACCTGGCACCTGCATCGC
CACTCTCAGAAGTTGGACTTGGATTTCTTTGTTTTGTTTTCGTCTGTTGCGGGTGTGCTGGGTAGCCC
TGGTCAAGGCAATTACGCGGCAGCCAACCGTGGCATGGCCGCCATCGCTCAGTACCGCCAGGCTCAAG
GTCTGCCGGCACTGGCGATTCACTGGGGCCCTTGGGCGGAAGGTGGTATGGCAAACAGCTTGAGCAAC
CAAAATCTGGCATGGTTGCCTCCGCCGCAGGGCTTGACCATTCTGGAAAAAGTTTTGGGTGCCCAAGG
CGAAATGGGCGTGTTCAAACCGGACTGGCAGAACTTGGCCAAACAATTCCCGGAGTTCGCGAAAACCC
ATTACTTTGCGGCGGTCATTCCGAGCGCTGAAGCGGTTCCACCGACCGCATCTATCTTCGACAAGCTG
ATCAATCTGGAAGCGAGCCAGCGCGCAGATTACCTGCTGGACTATCTGCGTAGATCTGTGGCACAAAT
TCTGAAACTGGAAATTGAGCAGATTCAGAGCCACGACTCCCTGCTGGATCTGGGTATGGATAGCCTGA
TGATCATGGAGGCGATTGCGTCCCTGAAACAAGACCTGCAACTGATGCTGTATCCGCGTGAGATTTAC
GAGCGTCCGCGTCTGGATGTTCTGACTGCTTACTTGGCCGCTGAGTTTACCAAAGCGCATGATTCTGA
AGCAGCTACCGCCGCAGCTGCGATCCCTAGCCAGAGCCTGAGCGTCAAAACCAAAAAGCAATGGCAGA
AACCGGATCATAAGAACCCGAATCCGATTGCGTTCATCCTGAGCAGCCCGCGTAGCGGTAGCACCCTG
CTGCGCGTGATGCTGGCCGGTCACCCGGGTCTGTATTCCCCACCGGAACTGCACCTGCTGCCGTTTGA
AACGATGGGTGACCGCCACCAGGAACTGGGTCTGTCTCATCTGGGCGAGGGTCTGCAACGTGCCCTGA
TGGACTTGGAAAATCTGACGCCGGAAGCATCCCAGGCAAAGGTGAACCAATGGGTGAAGGCGAATACG
CCGATTGCAGACATCTACGCATACCTGCAACGTCAAGCCGAGCAACGTCTGCTGATTGACAAAAGCCC
GAGCTATGGCAGCGACCGCCACATTCTGGATCACAGCGAGATCCTGTTCGATCAGGCGAAATACATCC
ACCTGGTTCGCCATCCTTATGCGGTCATTGAGAGCTTTACCCGCCTGCGTATGGACAAGCTGCTGGGT
GCAGAGCAACAGAATCCGTATGCGCTGGCGGAAAGCATTTGGCGTACCTCGAATCGCAACATTCTGGA
CTTGGGTCGTACCGTCGGCGCTGACCGCTACCTGCAAGTCATCTACGAGGATCTGGTGCGTGACCCGC
GTAAAGTTCTGACCAACATTTGTGATTTTCTGGGTGTCGATTTCGACGAGGCACTGCTGAATCCGTAC
TCCGGCGACCGCCTGACCGACGGCCTGCACCAGCAAAGCATGGGTGTGGGTGACCCGAACTTCTTGCA
GCACAAGACCATTGATCCGGCGCTAGCGGACAAATGGCGTAGCATTACCCTGCCGGCTGCTCTGCAAC
TGGATACGATTCAACTGGCCGAAACCTTCGCATACGACCTGCCGCAGGAGCCGCAGTTGACGCCGCAG
ACCCAATCTTTGCCATCGATGGTCGAACGTTTCGTCACGGTTCGCGGCCTGGAAACCTGTCTGTGCGA
GTGGGGTGATCGCCATCAACCTCTGGTCTTGCTGTTGCACGGTATCCTGGAGCAAGGCGCGTCTTGGC
AGTTGATCGCGCCTCAACTGGCAGCGCAGGGCTATTGGGTCGTCGCTCCGGATCTGCGCGGTCACGGT
AAATCTGCGCACGCGCAGTCTTATAGCATGCTGGATTTTCTGGCCGATGTGGACGCGCTGGCCAAACA
GTTGGGCGACCGTCCGTTCACCTTGGTTGGTCACAGCATGGGTTCCATCATTGGCGCAATGTATGCTG
GCATTCGTCAAACCCAGGTTGAAAAACTGATTCTGGTCGAAACCATCGTCCCGAATGATATTGATGAT
GCCGAAACCGGCAATCACCTGACCACCCATCTGGATTACCTGGCAGCCCCTCCGCAGCACCCGATCTT
TCCGAGCCTGGAAGTTGCGGCTCGTCGTCTGCGCCAAGCCACCCCGCAGTTGCCGAAAGACCTGTCTG
CATTTCTGACGCAACGTTCCACGAAGAGCGTCGAGAAGGGTGTGCAGTGGCGCTGGGATGCCTTCTTG
CGCACCCGTGCAGGTATCGAGTTTAACGGTATCAGCCGTCGCCGTTATCTGGCGCTGCTGAAAGATAT
CCAGGCCCCAATTACTTTGATTTACGGTGATCAGTCTGAGTTCAATCGCCCAGCAGACCTGCAAGCGA
TCCAGGCGGCACTGCCGCAAGCGCAACGCCTGACGGTTGCTGGCGGTCACAACTTGCACTTTGAGAAT
CCGCAGGCCATCGCCCAGATTGTCTATCAGCAGTTGCAGACACCGGTTCCGAAAACCCAAGGTTTGCA
CCATCACCACCATCATAGCGCCTGGAGCCACCCGCAGTTTGAAAAGTAA
SEQ ID NO: 3
nonA_optV6 (amino acid sequence)
MASWSHPQFEKEVHHHHHHGAVGQFANFVDLLQYRAKLQARKTVFSFLADGEAESAALTYGELDQKAQ
AIAAFLQANQAQGQRALLLYPPGLEFIGAFLGCLYAGVVAVPAYPPRPNKSFDRLHSIIQDAQAKFAL
TTTELKDKIADRLEALEGTDFHCLATDQVELISGKNWQKPNISGTDLAFLQYTSGSTGDPKGVMVSHH
NLIHNSGLINQGFQDTEASMGVSWLPPYHDMGLIGGILQPIYVGATQILMPPVAFLQRPFRWLKAIND
YRVSTSGAPNFAYDLCASQITPEQIRELDLSCWRLAFSGAEPIRAVTLENFAKTFATAGFQKSAFYPC
YGMAETTLIVSGGNGRAQLPQEIIVSKQGIEANQVRPAQGTETTVTLVGSGEVIGDQIVKIVDPQALT
ECTVGEIGEVWVKGESVAQGYWQKPDLTQQQFQGNVGAETGFLRTGDLGFLQGGELYITGRLKDLLII
RGRNHYPQDIELTVEVAHPALRQGAGAAVSVDVNGEEQLVIVQEVERKYARKLNVAAVAQAIRGAIAA
EHQLQPQAICFIKPGSIPKTSSGKIRRHACKAGFLDGSLAVVGEWQPSHQKEGKGIGTQAVTPSTTTS
TNFPLPDQHQQQIEAWLKDNIAHRLGITPQQLDETEPFASYGLDSVQAVQVTADLEDWLGRKLDPTLA
YDYPTIRTLAQFLVQGNQALEKIPQVPKIQGKEIAVVGLSCRFPQADNPEAFWELLRNGKDGVRPLKT
RWATGEWGGFLEDIDQFEPQFFGISPREAEQMDPQQRLLLEVTWEALERANIPAESLRHSQTGVFVGI
SNSDYAQLQVRENNPINPYMGTGNAHSIAANRLSYFLDLRGVSLSIDTACSSSLVAVHLACQSLINGE
SELAIAAGVNLILTPDVTQTFTQAGMMSKTGRCQTFDAEADGYVRGEGCGVVLLKPLAQAERDGDNIL
AVIHGSAVNQDGRSNGLTAPNGRSQQAVIRQALAQAGITAADLAYLEAHGTGTPLGDPIEINSLKAVL
QTAQREQPCVVGSVKTNIGHLEAAAGIAGLIKVILSLEHGMIPQHLHFKQLNPRIDLDGLVTIASKDQ
PWSGGSQKRFAGVSSFGFGGTNAHVIVGDYAQQKSPLAPPATQDRPWHLLTLSAKNAQALNALQKSYG
DYLAQHPSVDPRDLCLSANTGRSPLKERRFFVFKQVADLQQTLNQDFLAQPRLSSPAKIAFLFTGQGS
QYYGMGQQLYQTSPVFRQVLDECDRLWQTYSPEAPALTDLLYGNHNPDLVHETVYTQPLLFAVEYAIA
QLWLSWGVTPDFCMGHSVGEYVAACLAGVFSLADGMKLITARGKLMHALPSNGSMAAVFADKTVIKPY
LSEHLTVGAENGSHLVLSGKTPCLEASIHKLQSQGIKTKPLKVSHAFHSPLMAPMLAEFREIAEQITF
HPPRIPLISNVTGGQIEAEIAQADYWVKHVSQPVKFVQSIQTLAQAGVNVYLEIGVKPVLLSMGRHCL
AEQEAVWLPSLRPHSEPWPEILTSLGKLYEQGLNIDWQTVEAGDRRRKLILPTYPFQRQRYWFNQGSW
QTVETESVNPGPDDLNDWLYQVAWTPLDTLPPAPEPSAKLWLILGDRHDHQPIEAQFKNAQRVYLGQS
NHFPTNAPWEVSADALDNLFTHVGSQNLAGILYLCPPGEDPEDLDEIQKQTSGFALQLIQTLYQQKIA
VPCWFVTHQSQRVLETDAVTGFAQGGLWGLAQAIALEHPELWGGIIDVDDSLPNFAQICQQRQVQQLA
VRHQKLYGAQLKKQPSLPQKNLQIQPQQTYLVTGGLGAIGRKIAQWLAAAGAEKVILVSRRAPAADQQ
TLPTNAVVYPCDLADAAQVAKLFQTYPHIKGIFHAAGTLADGLLQQQTWQKFQTVAAAKMKGTWHLHR
HSQKLDLDFFVLFSSVAGVLGSPGQGNYAAANRGMAAIAQYRQAQGLPALAIHWGPWAEGGMANSLSN
QNLAWLPPPQGLTILEKVLGAQGEMGVFKPDWQNLAKQFPEFAKTHYFAAVIPSAEAVPPTASIFDKL
INLEASQRADYLLDYLRRSVAQILKLEIEQIQSHDSLLDLGMDSLMIMEAIASLKQDLQLMLYPREIY
ERPRLDVLTAYLAAEFTKAHDSEAATAAAAIPSQSLSVKTKKQWQKPDHKNPNPIAFILSSPRSGSTL
LRVMLAGHPGLYSPPELHLLPFETMGDRHQELGLSHLGEGLQRALMDLENLTPEASQAKVNQWVKANT
PIADIYAYLQRQAEQRLLIDKSPSYGSDRHILDHSEILFDQAKYIHLVRHPYAVIESFTRLRMDKLLG
AEQQNPYALAESIWRTSNRNILDLGRTVGADRYLQVIYEDLVRDPRKVLTNICDFLGVDFDEALLNPY
SGDRLTDGLHQQSMGVGDPNFLQHKTIDPALADKWRSITLPAALQLDTIQLAETFAYDLPQEPQLTPQ
TQSLPSMVERFVTVRGLETCLCEWGDRHQPLVLLLHGILEQGASWQLIAPQLAAQGYWVVAPDLRGHG
KSAHAQSYSMLDFLADVDALAKQLGDRPFTLVGHSMGSIIGAMYAGIRQTQVEKLILVETIVPNDIDD
AETGNHLTTHLDYLAAPPQHPIFPSLEVAARRLRQATPQLPKDLSAFLTQRSTKSVEKGVQWRWDAFL
RTRAGIEFNGISRRRYLALLKDIQAPITLIYGDQSEFNRPADLQAIQAALPQAQRLTVAGGHNLHFEN
PQAIAQIVYQQLQTPVPKTQGLHHHHHHSAWSHPQFEK
SEQ ID NO: 4
nonA (nucleotide sequence)
>SYNPCC7002_A1173 1-alkene synthase (PKS) [Synechococcus sp. PCC 7002]
Accession No: NC_010475.1 REGION: complement (1205897 . . . 1214059)
ATGGTTGGTCAATTTGCAAATTTCGTCGATCTGCTCCAGTACAGAGCTAAACTTCAGGCGCGGAAAACCG
TGTTTAGTTTTCTGGCTGATGGCGAAGCGGAATCTGCGGCCCTGACCTACGGAGAATTAGACCAAAAAGC
CCAGGCGATCGCCGCTTTTTTGCAAGCTAACCAGGCTCAAGGGCAACGGGCATTATTACTTTATCCACCG
GGTTTAGAGTTTATCGGTGCCTTTTTGGGATGTTTGTATGCTGGTGTTGTTGCGGTGCCAGCTTACCCAC
CACGGCCGAATAAATCCTTTGACCGCCTCCATAGCATTATCCAAGATGCCCAGGCAAAATTTGCCCTCAC
CACAACAGAACTTAAAGATAAAATTGCCGATCGCCTCGAAGCTTTAGAAGGTACGGATTTTCATTGTTTG
GCTACAGATCAAGTTGAATTAATTTCAGGAAAAAATTGGCAAAAACCGAACATTTCCGGCACAGATCTCG
CTTTTTTGCAATACACCAGTGGCTCCACGGGCGATCCTAAAGGAGTGATGGTTTCCCACCACAATTTGAT
CCACAACTCCGGCTTGATTAACCAAGGATTCCAGGATACAGAGGCGAGTATGGGCGTTTCCTGGTTGCCG
CCCTACCATGATATGGGCTTGATCGGTGGGATTTTACAGCCCATCTATGTGGGAGCAACGCAAATTTTAA
TGCCTCCCGTGGCCTTTTTGCAGCGACCTTTTCGGTGGCTAAAGGCGATCAACGATTATCGGGTTTCCAC
CAGCGGTGCGCCGAATTTTGCCTATGATCTCTGTGCCAGCCAAATTACCCCGGAACAAATCAGAGAACTC
GATTTGAGCTGTTGGCGACTGGCTTTTTCCGGGGCCGAACCGATCCGCGCTGTGACCCTCGAAAATTTTG
CGAAAACCTTCGCTACAGCAGGCTTTCAAAAATCAGCATTTTATCCCTGTTATGGTATGGCTGAAACCAC
CCTGATCGTTTCCGGTGGTAATGGTCGTGCCCAGCTTCCCCAGGAAATTATCGTCAGCAAACAGGGCATC
GAAGCAAACCAAGTTCGCCCTGCCCAAGGGACAGAAACAACGGTGACCTTGGTCGGCAGTGGTGAAGTGA
TTGGCGACCAAATTGTCAAAATTGTTGACCCCCAGGCTTTAACAGAATGTACCGTCGGTGAAATTGGCGA
AGTATGGGTTAAGGGCGAAAGTGTTGCCCAGGGCTATTGGCAAAAGCCAGACCTCACCCAGCAACAATTC
CAGGGAAACGTCGGTGCAGAAACGGGCTTTTTACGCACGGGCGATCTGGGTTTTTTGCAAGGTGGCGAAC
TGTATATTACGGGTCGTTTAAAGGATCTCCTGATTATCCGGGGGCGCAACCACTATCCCCAGGACATTGA
ATTAACCGTCGAAGTGGCCCATCCCGCTTTACGACAGGGGGCCGGAGCCGCTGTATCAGTAGACGTTAAC
GGGGAAGAACAGTTAGTCATTGTCCAGGAAGTTGAGCGTAAATATGCCCGCAAATTAAATGTCGCGGCAG
TAGCCCAAGCTATTCGTGGGGCGATCGCCGCCGAACATCAACTGCAACCCCAGGCCATTTGTTTTATTAA
ACCCGGTAGCATTCCCAAAACATCCAGCGGGAAGATTCGTCGCCATGCCTGCAAAGCTGGTTTTCTAGAC
GGAAGCTTGGCTGTGGTTGGGGAGTGGCAACCCAGCCACCAAAAAGAAGGAAAAGGAATTGGGACACAAG
CCGTTACCCCTTCTACGACAACATCAACGAATTTTCCCCTGCCTGACCAGCACCAACAGCAAATTGAAGC
CTGGCTTAAGGATAATATTGCCCATCGCCTCGGCATTACGCCCCAACAATTAGACGAAACGGAACCCTTT
GCAAGTTATGGGCTGGATTCAGTGCAAGCAGTACAGGTCACAGCCGACTTAGAGGATTGGCTAGGTCGAA
AATTAGACCCCACTCTGGCCTACGATTATCCGACCATTCGCACCCTGGCTCAGTTTTTGGTCCAGGGTAA
TCAAGCGCTAGAGAAAATACCACAGGTGCCGAAAATTCAGGGCAAAGAAATTGCCGTGGTGGGTCTCAGT
TGTCGTTTTCCCCAAGCTGACAACCCCGAAGCTTTTTGGGAATTATTACGTAATGGTAAAGATGGAGTTC
GCCCCCTTAAAACTCGCTGGGCCACGGGAGAATGGGGTGGTTTTTTAGAAGATATTGACCAGTTTGAGCC
GCAATTTTTTGGCATTTCCCCCCGGGAAGCGGAACAAATGGATCCCCAGCAACGCTTACTGTTAGAAGTA
ACCTGGGAAGCCTTGGAACGGGCAAATATTCCGGCAGAAAGTTTACGCCATTCCCAAACGGGGGTTTTTG
TCGGCATTAGTAATAGTGATTATGCCCAGTTGCAGGTGCGGGAAAACAATCCGATCAATCCCTACATGGG
GACGGGCAACGCCCACAGTATTGCTGCGAATCGTCTGTCTTATTTCCTCGATCTCCGGGGCGTTTCTCTG
AGCATCGATACGGCCTGTTCCTCTTCTCTGGTGGCGGTACATCTGGCCTGTCAAAGTTTAATCAACGGCG
AATCGGAGTTGGCGATCGCCGCCGGGGTGAATTTGATTTTGACCCCCGATGTGACCCAGACTTTTACCCA
GGCGGGCATGATGAGTAAGACGGGCCGTTGCCAGACCTTTGATGCCGAGGCTGATGGCTATGTGCGGGGC
GAAGGTTGTGGGGTCGTTCTCCTCAAACCCCTGGCCCAGGCAGAACGGGACGGGGATAATATTCTCGCGG
TGATCCACGGTTCGGCGGTGAATCAAGATGGACGCAGTAACGGTTTGACGGCTCCCAACGGGCGATCGCA
ACAGGCCGTTATTCGCCAAGCCCTGGCCCAAGCCGGCATTACCGCCGCCGATTTAGCTTACCTAGAGGCC
CACGGCACCGGCACGCCCCTGGGTGATCCCATTGAAATTAATTCCCTGAAGGCGGTTTTACAAACGGCGC
AGCGGGAACAGCCCTGTGTGGTGGGTTCTGTGAAAACAAACATTGGTCACCTCGAGGCAGCGGCGGGCAT
CGCGGGCTTAATCAAGGTGATTTTGTCCCTAGAGCATGGAATGATTCCCCAACATTTGCATTTTAAGCAG
CTCAATCCCCGCATTGATCTAGACGGTTTAGTGACCATTGCGAGCAAAGATCAGCCTTGGTCAGGCGGGT
CACAAAAACGGTTTGCTGGGGTAAGTTCCTTTGGGTTTGGTGGCACCAATGCCCACGTGATTGTCGGGGA
CTATGCTCAACAAAAATCTCCCCTTGCTCCTCCGGCTACCCAAGACCGCCCTTGGCATTTGCTGACCCTT
TCTGCTAAAAATGCCCAGGCCTTAAATGCCCTGCAAAAAAGCTATGGAGACTATCTGGCCCAACATCCCA
GCGTTGACCCACGCGATCTCTGTTTGTCTGCCAATACCGGGCGATCGCCCCTCAAAGAACGTCGTTTTTT
TGTCTTTAAACAAGTCGCCGATTTACAACAAACTCTCAATCAAGATTTTCTGGCCCAACCACGCCTCAGT
TCCCCCGCAAAAATTGCCTTTTTGTTTACGGGGCAAGGTTCCCAATACTACGGCATGGGGCAACAACTGT
ACCAAACCAGCCCAGTATTTCGGCAAGTGCTGGATGAGTGCGATCGCCTCTGGCAGACCTATTCCCCCGA
AGCCCCTGCCCTCACCGACCTGCTGTACGGTAACCATAACCCTGACCTCGTCCACGAAACTGTCTATACC
CAGCCCCTCCTCTTTGCTGTTGAATATGCGATCGCCCAACTATGGTTAAGCTGGGGCGTGACGCCAGACT
TTTGCATGGGCCATAGCGTCGGCGAATATGTCGCGGCTTGTCTGGCGGGGGTATTTTCCCTGGCAGACGG
CATGAAATTAATTACGGCCCGGGGCAAACTGATGCACGCCCTACCCAGCAATGGCAGTATGGCGGCGGTC
TTTGCCGATAAAACGGTCATCAAACCCTACCTATCGGAGCATTTGACCGTCGGAGCCGAAAACGGTTCCC
ATTTGGTGCTATCAGGAAAGACCCCCTGCCTCGAAGCCAGTATTCACAAACTCCAAAGCCAAGGGATCAA
AACCAAACCCCTCAAGGTTTCCCATGCTTTCCACTCCCCTTTGATGGCTCCCATGCTGGCAGAGTTTCGG
GAAATTGCTGAACAAATTACTTTCCACCCGCCGCGTATCCCGCTCATTTCCAATGTCACGGGCGGCCAGA
TTGAAGCGGAAATTGCCCAGGCCGACTATTGGGTTAAGCACGTTTCGCAACCCGTCAAATTTGTCCAGAG
CATCCAAACCCTGGCCCAAGCGGGTGTCAATGTTTATCTCGAAATCGGCGTAAAACCAGTGCTCCTGAGT
ATGGGACGCCATTGCTTAGCTGAACAAGAAGCGGTTTGGTTGCCCAGTTTACGTCCCCATAGTGAGCCTT
GGCCGGAAATTTTGACCAGTCTCGGCAAACTGTATGAGCAAGGGCTAAACATTGACTGGCAGACCGTGGA
AGCTGGCGATCGCCGCCGGAAACTGATTCTGCCCACCTATCCCTTCCAACGGCAACGATATTGGTTTAAT
CAAGGCTCTTGGCAAACTGTTGAGACCGAATCTGTGAACCCAGGCCCTGACGATCTCAATGATTGGTTGT
ATCAGGTGGCGTGGACGCCCCTGGACACTTTGCCCCCGGCCCCTGAACCGTCGGCTAAGCTGTGGTTAAT
CTTGGGCGATCGCCATGATCACCAGCCCATTGAAGCCCAATTTAAAAACGCCCAGCGGGTGTATCTCGGC
CAAAGCAATCATTTTCCGACGAATGCCCCCTGGGAAGTATCTGCCGATGCGTTGGATAATTTATTTACTC
ACGTCGGCTCCCAAAATTTAGCAGGCATCCTTTACCTGTGTCCCCCAGGGGAAGACCCAGAAGACCTAGA
TGAAATTCAAAAGCAAACCAGTGGCTTCGCCCTCCAACTGATCCAAACCCTGTATCAACAAAAGATCGCG
GTTCCCTGCTGGTTTGTGACCCACCAGAGCCAACGGGTGCTTGAAACCGATGCTGTCACCGGATTTGCCC
AAGGGGGATTATGGGGACTCGCCCAGGCGATCGCCCTCGAACATCCAGAGTTGTGGGGGGGAATTATTGA
TGTCGATGACAGCCTGCCAAATTTTGCCCAGATTTGCCAACAAAGACAGGTGCAGCAGTTGGCCGTGCGG
CACCAAAAACTCTACGGGGCACAGCTCAAAAAGCAACCGTCACTGCCCCAGAAAAATCTCCAGATTCAAC
CCCAACAGACCTATCTAGTGACAGGGGGACTGGGGGCCATTGGCCGTAAAATTGCCCAATGGCTAGCCGC
AGCAGGAGCAGAAAAAGTAATTCTCGTCAGCCGGCGCGCTCCGGCAGCGGATCAGCAGACGTTACCGACC
AATGCGGTGGTTTATCCTTGCGATTTAGCCGACGCAGCCCAGGTGGCAAAGCTGTTTCAAACCTATCCCC
ACATCAAAGGAATTTTCCATGCGGCGGGTACCTTAGCTGATGGTTTGCTGCAACAACAAACTTGGCAAAA
GTTCCAGACCGTCGCCGCCGCCAAAATGAAAGGGACATGGCATCTGCACCGCCATAGTCAAAAGCTCGAT
CTGGATTTTTTTGTGTTGTTTTCCTCTGTGGCAGGGGTGCTCGGTTCACCGGGACAGGGGAATTATGCCG
CCGCAAACCGGGGCATGGCGGCGATCGCCCAATATCGACAAGCCCAAGGTTTACCCGCCCTGGCGATCCA
TTGGGGGCCTTGGGCCGAAGGGGGAATGGCCAACTCCCTCAGCAACCAAAATTTAGCGTGGCTGCCGCCC
CCCCAGGGACTAACAATCCTCGAAAAAGTCTTGGGCGCCCAGGGGGAAATGGGGGTCTTTAAACCGGACT
GGCAAAACCTGGCCAAACAGTTCCCCGAATTTGCCAAAACCCATTACTTTGCAGCCGTTATTCCCTCTGC
TGAGGCTGTGCCCCCAACGGCTTCAATTTTTGACAAATTAATCAACCTAGAAGCTTCTCAGCGGGCTGAC
TATCTACTGGATTATCTGCGGCGGTCTGTGGCGCAAATCCTCAAGTTAGAAATTGAGCAAATTCAAAGCC
ACGATAGCCTGTTGGATCTGGGCATGGATTCGTTGATGATCATGGAGGCGATCGCCAGCCTCAAGCAGGA
TTTACAACTGATGTTGTACCCCAGGGAAATCTACGAACGGCCCAGACTTGATGTGTTGACGGCCTATCTA
GCGGCGGAATTCACCAAGGCCCATGATTCTGAAGCAGCAACGGCGGCAGCAGCGATTCCCTCCCAAAGCC
TTTCGGTCAAAACAAAAAAACAGTGGCAAAAACCTGACCACAAAAACCCGAATCCCATTGCCTTTATCCT
CTCTAGCCCCCGGTCGGGTTCGACGTTGCTGCGGGTGATGTTAGCCGGACATCCGGGGTTATATTCGCCG
CCAGAGCTGCATTTGCTCCCCTTTGAGACTATGGGCGATCGCCACCAGGAATTGGGTCTATCCCACCTCG
GCGAAGGGTTACAACGGGCCTTAATGGATCTAGAAAACCTCACCCCAGAGGCAAGCCAGGCGAAGGTCAA
CCAATGGGTCAAAGCGAATACACCCATTGCAGACATCTATGCCTATCTCCAACGGCAGGCGGAACAACGT
TTACTCATCGACAAATCTCCCAGCTACGGCAGCGATCGCCATATTCTAGACCACAGCGAAATCCTCTTTG
ACCAGGCCAAATATATCCATCTGGTACGCCATCCCTACGCGGTGATTGAATCCTTTACCCGACTGCGGAT
GGATAAACTGCTGGGGGCCGAGCAGCAGAACCCCTACGCCCTCGCGGAGTCCATTTGGCGCACCAGCAAC
CGCAATATTTTAGACCTGGGTCGCACGGTTGGTGCGGATCGATATCTCCAGGTGATTTACGAAGATCTCG
TCCGTGACCCCCGCAAAGTTTTGACAAATATTTGTGATTTCCTGGGGGTGGACTTTGACGAAGCGCTCCT
CAATCCCTACAGCGGCGATCGCCTTACCGATGGCCTCCACCAACAGTCCATGGGCGTCGGGGATCCCAAT
TTCCTCCAGCACAAAACCATTGATCCGGCCCTCGCCGACAAATGGCGCTCAATTACCCTGCCCGCTGCTC
TCCAGCTGGATACGATCCAGTTGGCCGAAACGTTTGCTTACGATCTCCCCCAGGAACCCCAGCTAACACC
CCAGACCCAATCCTTGCCCTCGATGGTGGAGCGGTTCGTGACAGTGCGCGGTTTAGAAACCTGTCTCTGT
GAGTGGGGCGATCGCCACCAACCATTGGTGCTACTTCTCCACGGCATCCTCGAACAGGGGGCCTCCTGGC
AACTCATCGCGCCCCAGTTGGCGGCCCAGGGCTATTGGGTTGTGGCCCCAGACCTGCGTGGTCACGGCAA
ATCCGCCCATGCCCAGTCCTACAGCATGCTTGATTTTTTGGCTGACGTAGATGCCCTTGCCAAACAATTA
GGCGATCGCCCCTTTACCTTGGTGGGCCACTCCATGGGTTCCATCATCGGTGCCATGTATGCAGGAATTC
GCCAAACCCAGGTAGAAAAGTTGATCCTCGTTGAAACCATTGTCCCCAACGACATCGACGACGCTGAAAC
CGGTAATCACCTGACGACCCATCTCGATTACCTCGCCGCGCCCCCCCAACACCCGATCTTCCCCAGCCTA
GAAGTGGCCGCCCGTCGCCTCCGCCAAGCCACGCCCCAACTACCCAAAGACCTCTCGGCGTTCCTCACCC
AGCGCAGCACCAAATCCGTCGAAAAAGGGGTGCAGTGGCGTTGGGATGCTTTCCTCCGTACCCGGGCGGG
CATTGAATTCAATGGCATTAGCAGACGACGTTACCTGGCCCTGCTCAAAGATATCCAAGCGCCGATCACC
CTCATCTATGGCGATCAGAGTGAATTTAACCGCCCTGCTGATCTCCAGGCGATCCAAGCGGCTCTCCCCC
AGGCCCAACGTTTAACGGTTGCTGGCGGCCATAACCTCCATTTTGAGAATCCCCAGGCGATCGCCCAAAT
TGTTTATCAACAACTCCAGACCCCTGTACCCAAAACACAATAA
SEQ ID NO: 5
nonA (amino acid sequence)
>gi|170077790|ref|YP_001734428.1| 1-alkene synthase [Synechococcus sp. PCC
7002]
Accession No: YP_001734428.1
MVGQFANFVDLLQYRAKLQARKTVFSFLADGEAESAALTYGELDQKAQAIAAFLQANQAQGQRALLLYPP
GLEFIGAFLGCLYAGVVAVPAYPPRPNKSFDRLHSIIQDAQAKFALTTTELKDKIADRLEALEGTDFHCL
ATDQVELISGKNWQKPNISGTDLAFLQYTSGSTGDPKGVMVSHHNLIHNSGLINQGFQDTEASMGVSWLP
PYHDMGLIGGILQPIYVGATQILMPPVAFLQRPFRWLKAINDYRVSTSGAPNFAYDLCASQITPEQIREL
DLSCWRLAFSGAEPIRAVTLENFAKTFATAGFQKSAFYPCYGMAETTLIVSGGNGRAQLPQEIIVSKQGI
EANQVRPAQGTETTVTLVGSGEVIGDQIVKIVDPQALTECTVGEIGEVWVKGESVAQGYWQKPDLTQQQF
QGNVGAETGFLRTGDLGFLQGGELYITGRLKDLLIIRGRNHYPQDIELTVEVAHPALRQGAGAAVSVDVN
GEEQLVIVQEVERKYARKLNVAAVAQAIRGAIAAEHQLQPQAICFIKPGSIPKTSSGKIRRHACKAGFLD
GSLAVVGEWQPSHQKEGKGIGTQAVTPSTTTSTNFPLPDQHQQQIEAWLKDNIAHRLGITPQQLDETEPF
ASYGLDSVQAVQVTADLEDWLGRKLDPTLAYDYPTIRTLAQFLVQGNQALEKIPQVPKIQGKEIAVVGLS
CRFPQADNPEAFWELLRNGKDGVRPLKTRWATGEWGGFLEDIDQFEPQFFGISPREAEQMDPQQRLLLEV
TWEALERANIPAESLRHSQTGVFVGISNSDYAQLQVRENNPINPYMGTGNAHSIAANRLSYFLDLRGVSL
SIDTACSSSLVAVHLACQSLINGESELAIAAGVNLILTPDVTQTFTQAGMMSKTGRCQTFDAEADGYVRG
EGCGVVLLKPLAQAERDGDNILAVIHGSAVNQDGRSNGLTAPNGRSQQAVIRQALAQAGITAADLAYLEA
HGTGTPLGDPIEINSLKAVLQTAQREQPCVVGSVKTNIGHLEAAAGIAGLIKVILSLEHGMIPQHLHFKQ
LNPRIDLDGLVTIASKDQPWSGGSQKRFAGVSSFGFGGTNAHVIVGDYAQQKSPLAPPATQDRPWHLLTL
SAKNAQALNALQKSYGDYLAQHPSVDPRDLCLSANTGRSPLKERRFFVFKQVADLQQTLNQDFLAQPRLS
SPAKIAFLFTGQGSQYYGMGQQLYQTSPVFRQVLDECDRLWQTYSPEAPALTDLLYGNHNPDLVHETVYT
QPLLFAVEYAIAQLWLSWGVTPDFCMGHSVGEYVAACLAGVFSLADGMKLITARGKLMHALPSNGSMAAV
FADKTVIKPYLSEHLTVGAENGSHLVLSGKTPCLEASIHKLQSQGIKTKPLKVSHAFHSPLMAPMLAEFR
EIAEQITFHPPRIPLISNVTGGQIEAEIAQADYWVKHVSQPVKFVQSIQTLAQAGVNVYLEIGVKPVLLS
MGRHCLAEQEAVWLPSLRPHSEPWPEILTSLGKLYEQGLNIDWQTVEAGDRRRKLILPTYPFQRQRYWFN
QGSWQTVETESVNPGPDDLNDWLYQVAWTPLDTLPPAPEPSAKLWLILGDRHDHQPIEAQFKNAQRVYLG
QSNHFPTNAPWEVSADALDNLFTHVGSQNLAGILYLCPPGEDPEDLDEIQKQTSGFALQLIQTLYQQKIA
VPCWFVTHQSQRVLETDAVTGFAQGGLWGLAQAIALEHPELWGGIIDVDDSLPNFAQICQQRQVQQLAVR
HQKLYGAQLKKQPSLPQKNLQIQPQQTYLVTGGLGAIGRKIAQWLAAAGAEKVILVSRRAPAADQQTLPT
NAVVYPCDLADAAQVAKLFQTYPHIKGIFHAAGTLADGLLQQQTWQKFQTVAAAKMKGTWHLHRHSQKLD
LDFFVLFSSVAGVLGSPGQGNYAAANRGMAAIAQYRQAQGLPALAIHWGPWAEGGMANSLSNQNLAWLPP
PQGLTILEKVLGAQGEMGVFKPDWQNLAKQFPEFAKTHYFAAVIPSAEAVPPTASIFDKLINLEASQRAD
YLLDYLRRSVAQILKLEIEQIQSHDSLLDLGMDSLMIMEAIASLKQDLQLMLYPREIYERPRLDVLTAYL
AAEFTKAHDSEAATAAAAIPSQSLSVKTKKQWQKPDHKNPNPIAFILSSPRSGSTLLRVMLAGHPGLYSP
PELHLLPFETMGDRHQELGLSHLGEGLQRALMDLENLTPEASQAKVNQWVKANTPIADIYAYLQRQAEQR
LLIDKSPSYGSDRHILDHSEILFDQAKYIHLVRHPYAVIESFTRLRMDKLLGAEQQNPYALAESIWRTSN
RNILDLGRTVGADRYLQVIYEDLVRDPRKVLTNICDFLGVDFDEALLNPYSGDRLTDGLHQQSMGVGDPN
FLQHKTIDPALADKWRSITLPAALQLDTIQLAETFAYDLPQEPQLTPQTQSLPSMVERFVTVRGLETCLC
EWGDRHQPLVLLLHGILEQGASWQLIAPQLAAQGYWVVAPDLRGHGKSAHAQSYSMLDFLADVDALAKQL
GDRPFTLVGHSMGSIIGAMYAGIRQTQVEKLILVETIVPNDIDDAETGNHLTTHLDYLAAPPQHPIFPSL
EVAARRLRQATPQLPKDLSAFLTQRSTKSVEKGVQWRWDAFLRTRAGIEFNGISRRRYLALLKDIQAPIT
LIYGDQSEFNRPADLQAIQAALPQAQRLTVAGGHNLHFENPQAIAQIVYQQLQTPVPKTQ
SEQ ID NO: 6
Synechococcus sp. PCC 7002 aoa locus (nucleotide sequence)
aoa locus: SYNPCC7002_A2265
Accession No: NC_010475.1: 2037569 . . . 2038552

| | |
|---|---|
| 1 | gtgcgcaaac cctggttaga acttcccttg gcgatttttt cctttggctt ttataaagtc |
| 61 | aacaaatttc tgattgggaa tctctacact ttgtatttag cgctgaataa aaaaaatgct |
| 121 | aaggaatggc gcattattgg agaaaaatcc ctccagaaat tcctgagttt acccgtttta |
| 181 | atgaccaaag cgccccggtg gaatacccac gccattatcg gcaccctggg accactctct |
| 241 | gtagaaaaag aactcaccat taacctcgaa acgattcgtc aatccacgga agcttgggtc |
| 301 | ggttgcatct atgactttcc gggctatcgc acggtgttaa atttcacgca actcaccgat |
| 361 | gaccccaacc aaacagaact caaaattttc ttacctaaag ggaaatatac cgtcgggtta |
| 421 | cgttactacc atcccaaggt aaatcctcgc tttccggtcg ttaaaacaga tctaaatcta |
| 481 | accgtgccga ctttggttgt ttcgccccaa aacaacgact tttatcaagc cctggcccag |
| 541 | aaaacaaacc tttattttcg tctgcttcac tactacattt ttacgctatt taaatttcgc |
| 601 | gatgtcttac ccgctgcttt tgtgaaagga gaattcctcc ctgtcggcgc caccgatact |
| 661 | caattttttt acggcgcttt agaagcagca gaaaacttag agattaccat cccagccccc |
| 721 | tggcttcaga cctttgattt ttatctcacc ttctataacc gcgccagttt tcccctacgt |
| 781 | tggcaaaaaa tcaccgaagc gatgatctgt gatcccctgg gagaaaaagg ctattaccta |
| 841 | attcggatgc ggccccgtac tcaggacgcc gaggcacaat taccaacggt tagaggagaa |
| 901 | gaaacccagg tcacgcccca gcagaaaaaa ctggcgatcc agtccctata a |

SEQ ID NO: 7
Synechococcus sp. PCC 7002 aoa locus (amino acid sequence)
aoa locus: SYNPCC7002_A2265
AccessionNo:YP_001735499.1

| | |
|---|---|
| 1 | MRKPWLELPL AIFSFGFYKV NKFLIGNLYT LYLALNKKNA KEWRIIGEKS LQKFLSLPVL |
| 61 | MTKAPRWNTH AIIGTLGPLS VEKELTINLE TIRQSTEAWV GCIYDFPGYR TVLNFTQLTD |
| 121 | DPNQTELKIF LPKGKYTVGL RYYHPKVNPR FPVVKTDLNL TVPTLVVSPQ NNDFYQALAQ |
| 181 | KTNLYFRLLH YYIFTLFKFR DVLPAAFVKG EFLPVGATDT QFFYGALEAA ENLEITIPAP |
| 241 | WLQTFDFYLT FYNRASFPLR WQKITEAMIC DPLGEKGYYL IRMRPRTQDA EAQLPTVRGE |
| 301 | ETQVTPQQKK LAIQSL |

SEQ ID NO: 8
Cyanothece sp. PCC 7822 aoa locus (nucleotide sequence)
aoa locus: Cyan7822_1848
Accession No: NC_014501.1: 2037569 . . . 2038552

| | |
|---|---|
| 1 | atgacccaaa aaacatcaac aatttttgaa atccccttgg ctttgttatc cttcttattt |
| 61 | tacaaagcca tgaaattcct catcggcaat ctttacacaa tctatttaac ttttaataaa |
| 121 | agtaaagcct cacaatggcg agtcctatct gaagaagtcg tgatcaaaac cgccctcagc |
| 181 | ttaccggttt taatgacaaa aggtcctcgc tggaataccc acgccatcat cggaaccctt |
| 241 | gggcccttta atgttaatca atctattgct attgatttaa attcagctaa tcaaactact |
| 301 | cgatcctgga tcgccgttat ttatagtttt ccagggtatg aaactatcgc gagtcttgaa |
| 361 | tcaaatcgca ttaaccctca agaacaatgg gcatctttag ccttaaaacc cggtaaatat |
| 421 | agtatcggat tgagatatta taattggggt gaaaaagtga ttgttccaac ggttaaagtg |
| 481 | gatgatcaga tatttgtaga atctcaatcg attccttcag atattaataa gttttattta |
| 541 | gatttaattc agaaaaaaaa ttggttttat ttaagtcttc attattatat ttttaccctg |
| 601 | ttgcggctga gaaagcggct accagaatcc ttgataaaac aggaatattt accggttggg |
| 661 | gcaacggata ctgaatttgt ctataattat ttaacccgag gacaggcgct acaaatttct |
| 721 | cttgattccg acttagttaa gaattatgac atttacttga caatttatga tcgttcgagt |
| 781 | ttaccgttaa cttggagcca aattacagaa gaaaactatt taacgaaacc tatcgaaaac |
| 841 | aacggctatt atttaattcg gatgcgccct aaatatgtct cgttagaaga agtgttaaaa |
| 901 | cagttaccgg ttcagtctgt aataagcgat gaagagacgt tgactcaaaa gcttaagcta |
| 961 | accgttaaaa ccggtcaaaa ttaa |

SEQ ID NO: 9
Cyanothece sp. PCC 7822 aoa locus (amino acid sequence)
aoa locus: Cyan7822_1848
Accession No: YP_003887108.1

| | |
|---|---|
| 1 | MTQKTSTIFE IPLALLSFLF YKAMKFLIGN LYTIYLTFNK SKASQWRVLS EEVVIKTALS |
| 61 | LPVLMTKGPR WNTHAIIGTL GPFNVNQSIA IDLNSANQTT RSWIAVIYSF PGYETIASLE |
| 121 | SNRINPQEQW ASLALKPGKY SIGLRYYNWG EKVIVPTVKV DDQIFVESQS IPSDINKFYL |
| 181 | DLIQKKNWFY LSLHYYIFTL LRLRKRLPES LIKQEYLPVG ATDTEFVYNY LTRGQALQIS |
| 241 | LDSDLVKNYD IYLTIYDRSS LPLTWSQITE ENYLTKPIEN NGYYLIRMRP KYVSLEEVLK |
| 301 | QLPVQSVISD EETLTQKLKL TVKTGQN |

SEQ ID NO: 10
Cyanothece sp. PCC 7424 aoa locus (nucleotide sequence)
aoa locus: PCC7424_1874
Accession No: NC_011729A: 209923. . . 2100912

| | |
|---|---|
| 1 | atgagtagtc aattttccaa attatctatt gttgaactct ttttagaatt gcccttgact |
| 61 | ttgttatctt ttgtttttta caaagtcatg aaatttatga ttggcaattt atatacagtc |
| 121 | tatttaacct ttaataaaag taaaacatct caatggcgag tcttatcaga agaggtaatt |
| 181 | aaatctgccc tcagtgtacc ggttttaatg actaaagggc ctcgttggaa tactcatgct |
| 241 | attattggaa cacttggccc tttttccgtt aatcaatcta ttgctattga tttaaattca |
| 301 | gttaatcaaa cctctcaatc ttggattgcc gttatttata actttcccca atatgaaacc |
| 361 | attaccagtt tagaatcaaa ccgaattaat tccgataatc aatgggcttg tttgacctta |
| 421 | aaaccgggga aatatagtat aggattgaga tattataact ggggagaaaa ggttgttttt |
| 481 | ccctcgataa aagttgagga taaagttttt gttgatcctc aagttatccc ctcagaagtg |
| 541 | aatcagtttt attcgagttt aattaattat aaaaactggt tttatttaag tcttcattat |
| 601 | tatattttta ccctgttgag attgagaaaa attttgccag attcttttgt caaacaggaa |
| 661 | tatttacccg ttggggcaac ggatacggaa tttgtctata attatttact caaagggcaa |
| 721 | gccttacaaa ttacccttga ctcagaatta gttaagaatt atgacattta cttgacaatt |
| 781 | tatgatcggt ctagtttgcc cttaagttgg gatcggatca tagaagacaa gtatttaaca |
| 841 | aaaccgatag aaaacaacgg atattattta attcggatgc ggcctaaata tacctcctta |
| 901 | gaagaaatct taacagagtt accagttgag tctcaaatca gtgatgaaac cgaattaatt |
| 961 | caacagctta aattaaaagt taaaggctaa |

SEQ ID NO: 11
Cyanothece sp. PCC 7424 aoa locus (amino acid sequence)
aoa locus: PCC7424_1874
Accession No: YP_002377175

| | |
|---|---|
| 1 | MSSQFSKLSI VELFLELPLT LLSFVFYKVM KFMIGNLYTV YLTFNKSKTS QWRVLSEEVI |
| 61 | KSALSVPVLM TKGPRWNTHA IIGTLGPFSV NQSIAIDLNS VNQTSQSWIA VIYNFPQYET |
| 121 | ITSLESNRIN SDNQWACLTL KPGKYSIGLR YYNWGEKVVF PSIKVEDKVF VDPQVIPSEV |
| 181 | NQFYSSLINY KNWFYLSLHY YIFTLLRLRK ILPDSFVKQE YLPVGATDTE FVYNYLLKGQ |
| 241 | ALQITLDSEL VKNYDIYLTI YDRSSLPLSW DRIIEDKYLT KPIENNGYYL IRMRPKYTSL |
| 301 | EEILTELPVE SQISDETELI QQLKLKVKG |

SEQ ID NO: 12
Lyngbya majuscule 3L aoa locus (nucleotide sequence)
aoa locus: LYNGBM3L_11290
Accession No.: NZ_GL890825: 317925 . . . 318770

| | |
|---|---|
| 1 | atgcaaacca tcggaggata ctttacctcc aaaaaaaaca ctaaaaatct ccagtggcaa |
| 61 | ctcgtatcag ccgagttttt aaaaaagccc atcaaattaa tttgggcaat gagtcgagct |
| 121 | cgttggaatc ttcacgctat tatttctcta gttggaccga ttcaggtcaa agagctaatt |
| 181 | agctttgatg ccagtgcagc taaacaatca gcccaatcct ggacattagt agtttacagt |
| 241 | ctaccagatt ttgaaaccat cactaatatc agctccctga ccgtatccgg agaaaaccaa |
| 301 | tgggaatccg tgatcttaaa accaggtaaa tacttattag gtttgcggta ttatcactgg |
| 361 | tcagagacag tagagcaacc tactgttaaa gcagatggtg ttaaagtcgt agatgccaag |
| 421 | caaattcacg cccctactga tatcaacagc ttttaccgtg acctaattaa acgaaaaaat |
| 481 | tggcttcatg tctggttaaa ttattatgtc ttcaacctgt tgcactttaa gcaatggtta |
| 541 | ccccaggcat ttgttaaaaa agtattctta cctgtaccga atccagaaac caaattttac |
| 601 | tatggtgcct tgaaaaaggg agaatcgatt caatttaaac tagcaccatc cttgttaaca |
| 661 | agccatgatc tttactacag cttgtacagc cgtgaatgct ttccgctaga ttggtacaaa |
| 721 | attactgaag gggaacatag aacatctgct agtgagcaga agtctattta tattgttcgg |
| 781 | attcatccga aatttgagcg aaacgcttta tttgaaaata gttgggtgaa gatagccgtt |
| 841 | gtttga |

SEQ ID NO: 13
Lyngbya majuscule 3L aoa locus (amino acid sequence)
aoa locus: LYNGBM3L_11290
Accession No: ZP_08425909.1

| | |
|---|---|
| 1 | MQTIGGYFTS KKNTKNLQWQ LVSAEFLKKP IKLIWAMSRA RWNLHAIISL VGPIQVKELI |
| 61 | SFDASAAKQS AQSWTLVVYS LPDFETITNI SSLTVSGENQ WESVILKPGK YLLGLRYYHW |
| 121 | SETVEQPTVK ADGVKVVDAK QIHAPTDINS FYRDLIKRKN WLHVWLNYYV FNLLHFKQWL |
| 181 | PQAFVKKVFL PVPNPETKFY YGALKKGESI QFKLAPSLLT SHDLYYSLYS RECFPLDWYK |
| 241 | ITEGEHRTSA SEQKSIYIVR IHPKFERNAL FENSWVKIAV V |

SEQ ID NO: 14
Lyngbya majuscule 3L aoa locus (nucleotide sequence)
aoa locus: LYNGBM3L_74520
Accession No: NZ_GL890975: 5456 . . . 6466 (complement)

| | |
|---|---|
| 1 | atggaaacta aagaaaaatt tttattcttc caactctggt gggaaattcc actagcattg |
| 61 | ttatctttga tattttataa agctgttaag ggacttatac ccattctttt tcaaaagaaa |
| 121 | accaaaacca agaaaaaaat agcagactta accaaaaaag aagtttataa atggcgattt |
| 181 | gtttctgaag aactgctaaa acagcctctg gtactatcct atattttaac tactggtcct |
| 241 | cgatggaatg tccacgccat tattgccact acagaaccgg ttccagtcaa agaatcatta |
| 301 | aaaattgata tcagttcttg tgtggcttca gctcagtcat ggagtatagg tatctatagt |
| 361 | tttcctgaag gcaaacctgt caaatacata gcatctcatg agccaaaatt tcataaacaa |
| 421 | tggcaagaaa tcaaactgga accgggaaaa tataatttag ctttaagata ttataattgg |
| 481 | tacgatcaag tcagtttacc tgctgttatt atggataata atcaaattat caatactgaa |
| 541 | tcagttaata gtagtcagat taacaattac ttcaattatt tgcccaaatt aataggacaa |
| 601 | gataatattt tttatcgatt tcttaattac tatatattca ctattctagt atgccagaaa |
| 661 | tggctaccta aagaatgggt tagaaaagaa tttttacctg tgggagaccc caataatgag |
| 721 | tttgtctatg gagttattta taaaggttac tatttggctc tgacattaaa tccattatta |
| 781 | ctcactaatt atgatgttta tttaaccaca tacaatcgtt ctagtctacc aattaatttt |
| 841 | tgtcaaatta atactgacaa atacacaact tctgtgatag aaaccgacgg tttttattta |
| 901 | gtgcgattgc gtcctaagtc agatttagac aataatttat ttcagctaaa ttggattagt |
| 961 | acagagcttg tatcagaagt ttcctgtaac cgttcagggg gcgaagtctg a |

SEQ ID NO: 15
Lyngbya majuscule 3L aoa locus (amino acid sequence)
aoa locus: LYNGBM3L_74520
Accession No: ZP_08432358

| | |
|---|---|
| 1 | METKEKFLFF QLWWEIPLAL LSLIFYKAVK GLIPILFQKK TKTKKKIADL TKKEVYKWRF |
| 61 | VSEELLKQPL VLSYILTTGP RWNVHAIIAT TEPVPVKESL KIDISSCVAS AQSWSIGIYS |
| 121 | FPEGKPVKYI ASHEPKFHKQ WQEIKLEPGK YNLALRYYNW YDQVSLPAVI MDNNQIINTE |
| 181 | SVNSSQINNY FNYLPKLIGQ DNIFYRFLNY YIFTILVCQK WLPKEWVRKE FLPVGDPNNE |
| 241 | FVYGVIYKGY YLALTLNPLL LTNYDVYLTT YNRSSLPINF CQINTDKYTT SVIETDGFYL |
| 301 | VRLRPKSDLD NNLFQLNWIS TELVSEVSCN RSGGEV |

SEQ ID NO: 16
Haliangium ochraceum DSM 14365 aoa locus (nucleotide sequence)
aoa locus: Hoch_0800
Accession No: NC_013440.1: 1053227 . . . 1054147

| | |
|---|---|
| 1 | atgcgccgta gtcgtctgtt gctcgaggcc cccctcgcgc tcgcctcctt cgccctcaac |
| 61 | cgcgcggccc tggcgcgcgc cctgaagccg atgagtcgcg cgcccgccag cgaccaaccg |
| 121 | cgcgcgtgga agctcatgga cgaggcgttc tttgccccgc cttcggtcat gacagcgtac |
| 181 | tcgctgctgg cgccgcgatg gaacgtgcac gcggccatcg cggtctcgcc gattcttccc |
| 241 | gtgaccggac gcgtgtccgt cgacgtcgcc gctgccaacg cagcatcccc gcgttggacg |
| 301 | ctcgtcgcct acgacaagca agggacggtc gccgccgtcg gcaccacaaa caccgaagca |
| 361 | gacgcatcct gggccgccat cgagctgtcg cccggactgt atcgcttcgt gattcgcctc |
| 421 | tacgagcccg ggcccggcgg ggtggtcccc gaagtccata tcgatggcga gccggcgctc |
| 481 | gccgcattgg agctgccaga agacccgact cgtgtgtatc ggagcctgcg cgcccgcggc |
| 541 | gggcggaggc accgagcgtt gcagcgatac gtctatccca tggtgcggct gcggcggctc |
| 601 | ctcggcgagg agcgcgtgac ccgcgagtac ttaccggtgg gaaaccccga gaccctgttt |
| 661 | cgctttggcg tggtcgagcg cggtcagcgg ctcgaactcc gcccgcccga cgaattaccc |
| 721 | gatgattgcg gcctgtatct atgcctatac gatcagtcga gtctgcccat gtggttcggg |
| 781 | ccaatcctgc ccgagggcat acagacgccg cctgcgccgg accacggcac ctggctcgtc |
| 841 | cgcatcgtgc ccgggcggca tggcgcgccg gatccggcac ggattcaggt tcgcgtaatg |
| 901 | tccgaaaagc cgatcgcgta a |

SEQ ID NO: 17
Haliangium ochraceum DSM 14365 aoa locus (amino acid sequence)
aoa locus: Hoch_0800
Accession No: YP_003265309

| | |
|---|---|
| 1 | MRRSRLLLEA PLALASFALN RAALARALKP MSRAPASDQP RAWKLMDEAF FAPPSVMTAY |
| 61 | SLLAPRWNVH AAIAVSPILP VTGRVSVDVA AANAASPRWT LVAYDKQGTV AAVGTTNTEA |
| 121 | DASWAAIELS PGLYRFVIRL YEPGPGGVVP EVHIDGEPAL AALELPEDPT RVYRSLRARG |
| 181 | GRRHRALQRY VYPMVRLRRL LGEERVTREY LPVGNPETLF RFGVVERGQR LELRPPDELP |
| 241 | DDCGLYLCLY DQSSLPMWFG PILPEGIQTP PAPDHGTWLV RIVPGRHGAP DPARIQVRVM |
| 301 | SEKPIA |

SEQ ID NO: 18
Synechococcus sp. PCC 7002 aoa (Genbank NC_010475, locus A2265) modified to
contain a C-terminal Strep-tag II and His tag (nucleotide sequence)
ATGCGCAAACCCTGGTTAGAACTTCCCTTGGCGATTTTTTCCTTTGGCTTTTATAAAGTCAACAAATTT
CTGATTGGGAATCTCTACACTTTGTATTTAGCGCTGAATAAAAAAAATGCTAAGGAATGGCGCATTATT
GGAGAAAAATCCCTCCAGAAATTCCTGAGTTTACCCGTTTTAATGACCAAAGCGCCCCGGTGGAATACC
CACGCCATTATCGGCACCCTGGGACCACTCTCTGTAGAAAAAGAACTCACCATTAACCTCGAAACGATT
CGTCAATCCACGGAAGCTTGGGTCGGTTGCATCTATGACTTTCCGGGCTATCGCACGGTGTTAAATTTC
ACGCAACTCACCGATGACCCCAACCAAACAGAACTCAAAATTTTCTTACCTAAAGGGAAATATACCGTC
GGGTTACGTTACTACCATCCCAAGGTAAATCCTCGCTTTCCGGTCGTTAAAACAGATCTAAATCTAACC
GTGCCGACTTTGGTTGTTTCGCCCCAAAACAACGACTTTTATCAAGCCCTGGCCCAGAAAACAAACCTT
TATTTTCGTCTGCTTCACTACTACATTTTTACGCTATTTAAATTTCGCGATGTCTTACCCGCTGCTTTT
GTGAAAGGAGAATTCCTCCCTGTCGGCGCCACCGATACTCAATTTTTTTACGGCGCTTTAGAAGCAGCA
GAAAACTTAGAGATTACCATCCCAGCCCCCTGGCTTCAGACCTTTGATTTTTATCTCACCTTCTATAAC
CGCGCCAGTTTTCCCCTACGTTGGCAAAAAATCACCGAAGCGATGATCTGTGATCCCCTGGGAGAAAAA
GGCTATTACCTAATTCGGATGCGGCCCCGTACTCAGGACGCCGAGGCACAATTACCAACGGTTAGAGGA
GAAGAAACCCAGGTCACGCCCCAGCAGAAAAAACTGGCGATCCAGTCCCTAGGTTTGCACCATCACCAC
CATCATAGCGCCTGGAGCCACCCGCAGTTTGAAAAGTAA
SEQ ID NO: 19
Synechococcus sp. PCC 7002 aoa (Genbank NC_010475, locus A2265) modified to
contain a C-terminal Strep-tag II and His tag (amino acid sequence)
MRKPWLELPLAIFSFGFYKVNKFLIGNLYTLYLALNKKNAKEWRIIGEKSLQKFLSLPVLMTKAPRWNT
HAIIGTLGPLSVEKELTINLETIRQSTEAWVGCIYDFPGYRTVLNFTQLTDDPNQTELKIFLPKGKYTV
GLRYYHPKVNPRFPVVKTDLNLTVPTLVVSPQNNDFYQALAQKTNLYFRLLHYYIFTLFKFRDVLPAAF
VKGEFLPVGATDTQFFYGALEAAENLEITIPAPWLQTFDFYLTFYNRASFPLRWQKITEAMICDPLGEK
GYYLIRMRPRTQDAEAQLPTVRGEETQVTPQQKKLAIQSLGLHHHHHHSAWSHPQFEK
SEQ ID NO: 20
tsr2142 promoter (nucleotide sequence)
ATGATCAGGAGGAGTCTTTTTTGAGTGCTAGCTCCCCTGACGCAGGGTCACTCTTGTAAGTTCCAGTAG
CACTCTTTTGGCAAGCATTGAAGCATTCAAACCAGTGAAATCCCCTCGCTGGAGCAGCGAAGTTTAAGC
TATCGTTGAAGTAGCCACCTTGG
SEQ ID NO: 21
ompR promoter (nucleotide sequence)
TAGTACAAAAAGACGATTAACCCCATGGGTAAAAGCAGGGGAGCCACTAAAGTTCACAGGTTTACACCG
AATTTTCCATTTGAAAAGTAGTAAATCATACAGAAAACAATCATGTAAAAATTGAATACTCTAATGGTT
TGATGTCCGAAAAAGTCTAGTTTCTTCTATTCTTCGACCAAATCTATGGCAGGGCACTATCACAGAGCT
GGCTTAATAATTTGGGAGAAATGGGTGGGGGCGGACTTTCGTAGAACAATGTAGATTAAAGTACTGTAC
AT
SEQ ID NO: 22
aadA coding sequence (spectinomycin selection marker) (nucleotide sequence)
ATGAGGGAAGCGGTGATCGCCGAAGTATCGACTCAACTATCAGAGGTAGTTGGCGTCATCGAGCGCCAT
CTCGAACCGACGTTGCTGGCCGTACATTTGTACGGCTCCGCAGTGGATGGCGGCCTGAAGCCACACAGT
GATATTGATTTGCTGGTTACGGTGACCGTAAGGCTTGATGAAACAACGCGGCGAGCTTTGATCAACGAC
CTTTTGGAAACTTCGGCTTCCCCTGGAGAGAGCGAGATTCTCCGCGCTGTAGAAGTCACCATTGTTGTG
CACGACGACATCATTCCGTGGCGTTATCCAGCTAAGCGCGAACTGCAATTTGGAGAATGGCAGCGCAAT
GACATTCTTGCAGGTATCTTCGAGCCAGCCACGATCGACATTGATCTGGCTATCTTGCTGACAAAAGCA
AGAGAACATAGCGTTGCCTTGGTAGGTCCAGCGGCGGAGGAACTCTTTGATCCGGTTCCTGAACAGGAT
CTATTTGAGGCGCTAAATGAAACCTTAACGCTATGGAACTCGCCGCCCGACTGGGCTGGCGATGAGCGA
AATGTAGTGCTTACGTTGTCCCGCATTTGGTACAGCGCAGTAACCGGCAAAATCGCGCCGAAGGATGTC
GCTGCCGACTGGGCAATGGAGCGCCTGCCGGCCCAGTATCAGCCCGTCATACTTGAAGCTAGACAGGCT
TATCTTGGACAAGAAGAAGATCGCTTGGCCTCGCGCGCAGATCAGTTGGAAGAATTTGTCCACTACGTG
AAAGGCGAGATCACCAAGGTAGTCGGCAAATAA
SEQ ID NO: 23
plasmid pJB2580 (nucleotide sequence)

| | |
|---|---|
| 1^{st} underlined sequence | Upstream homology region for SYNPCC7002_A0358 |
| 1^{st} italic sequence | aoaH6SII coding sequence |
| 1^{st} bold sequence | tsr2142 promoter |
| 2^{nd} bold sequence | ompR promoter |
| 2^{nd} italic sequence | nonA_optV6 coding sequence |
| 2^{nd} underlined sequence | aadA coding sequence; spectinomycin selection marker |
| 3^{rd} bold sequence | Downstream homology region for |

SYNPCC7002_A0358
TTAGAAAAACTCATCGAGCATCAAATGAAACTGCAATTTATTCATATCAGGATTATCAATACCATATTT
TTGAAAAAGCCGTTTCTGTAATGAAGGAGAAAACTCACCGAGGCAGTTCCATAGGATGGCAAGATCCTG
GTATCGGTCTGCGATTCCGACTCGTCCAACATCAATACAACCTATTAATTTCCCCTCGTCAAAAATAAG
GTTATCAAGTGAGAAATCACCATGAGTGACGACTGAATCCGGTGAGAATGGCAAAAGTTTATGCATTTC
TTTCCAGACTTGTTCAACAGGCCAGCCATTACGCTCGTCATCAAAATCACTCGCATCAACCAAACCGTT
ATTCATTCGTGATTGCGCCTGAGCGAGGCGAAATACGCGATCGCTGTTAAAAGGACAATTACAAACAGG
AATCGAGTGCAACCGGCGCAGGAACACTGCCAGCGCATCAACAATATTTTCACCTGAATCAGGATATTC
TTCTAATACCTGGAACGCTGTTTTTCCGGGGATCGCAGTGGTGAGTAACCATGCATCATCAGGAGTACG
GATAAAATGCTTGATGGTCGGAAGTGGCATAAATTCCGTCAGCCAGTTTAGTCTGACCATCTCATCTGT
AACATCATTGGCAACGCTACCTTTGCCATGTTTCAGAAACAACTCTGGCGCATCGGGCTTCCCATACAA
GCGATAGATTGTCGCACCTGATTGCCCGACATTATCGCGAGCCCATTTATACCCATATAAATCAGCATC
CATGTTGGAATTTAATCGCGGCCTCGACGTTTCCCGTTGAATATGGCTCATATTCTTCCTTTTTCAATA
TTATTGAAGCATTTATCAGGGTTATTGTCTCATGAGCGGATACATATTTGAATGTATTTAGAAAAATAA
ACAAATAGGGGTCAGTGTTACAACCAATTAACCAATTCTGAACATTATCGCGAGCCCATTTATACCTGA
ATATGGCTCATAACACCCCTTGTTTGCCTGGCGGCAGTAGCGCGGTGGTCCCACCTGACCCCATGCCGA
ACTCAGAAGTGAAACGCCGTAGCGCCGATGGTAGTGTGGGGACTCCCCATGCGAGAGTAGGGAACTGCC
AGGCATCAAATAAAACGAAAGGCTCAGTCGAAAGACTGGGCCTTTCGCCCGGGCTAATTAGGGGGTGTC
GCCCTTATTCGACTCTATAGTGAAGTTCCTATTCTCTAGAAAGTATAGGAACTTCTGAAGTGGGGCCTG
CAGGACAACTCGGCTTCCGAGCTTGGCTCCACCATGGTTATATCTGGAGTAACCAGAATTTCGACAACT
TCGACGACTATCTCGGTGCTTTTACCTCCAACCAACGCAAAAACATTAAGCGCGAACGCAAAGCCGTTG
ACAAAGCAGGTTTATCCCTCAAGATGATGACCGGGGACGAAATTCCCGCCCATTACTTCCCACTCATTT
ATCGTTTCTATAGCAGCACCTGCGACAAATTTTTTTGGGGGAGTAAATATCTCCGGAAACCCTTTTTTG
AAACCCTAGAATCTACCTATCGCCATCGCGTTGTTCTGGCCGCCGCTTACACGCCAGAAGATGACAAAC
ATCCCGTCGGTTTATCTTTTTGTATCCGTAAAGATGATTATCTTTATGGTCGTTATTGGGGGGCCTTTG
ATGAATATGACTGTCTCCATTTTGAAGCCTGCTATTACAAACCGATCCAATGGGCAATCGAGCAGGGAA
TTACGATGTACGATCCGGGCGCTGGCGGAAAACATAAGCGACGACGTGGTTTCCCGGCAACCCCAAACT
ATAGCCTCCACCGTTTTTATCAACCCCGCATGGGCCAAGTTTTAGACGCTTATATTGATGAAATTAATG
CCATGGAGCAACAGGAAATTGAAGCGATCAATGCGGATATTCCCTTTAAACGGCAGGAAGTTCAATTGA
AAATTTCCTAGCTTCACTAGCCAAAAGCGCGATCGCCCACCGACCATCCTCCCTTGGGGGAGATGCGGC
CGCGCGAAAAAACCCCGCCGAAGCGGGGTTTTTTGCGGACGTCTTACTTTTCAAACTGCGGGTGGCTCC
AGGCGCTATGATGGTGGTGATGGTGCAAACCTAGGGACTGGATCGCCAGTTTTTTCTGCTGGGGCGTGA
CCTGGGTTTCTTCTCCTCTAACCGTTGGTAATTGTGCCTCGGCGTCCTGAGTACGGGGCCGCATCCGAA
TTAGGTAATAGCCTTTTTCTCCCAGGGGATCACAGATCATCGCTTCGGTGATTTTTTGCCAACGTAGGG
GAAAACTGGCGCGGTTATAGAAGGTGAGATAAAAATCAAAGGTCTGAAGCCAGGGGGCTGGGATGGTAA
TCTCTAAGTTTTCTGCTGCTTCTAAAGCGCCGTAAAAAAATTGAGTATCGGTGGCGCCGACAGGGAGGA
ATTCTCCTTTCACAAAAGCAGCGGGTAAGACATCGCGAAATTTAAATAGCGTAAAAATGTAGTAGTGAA
GCAGACGAAAATAAAGGTTTGTTTTCTGGGCCAGGGCTTGATAAAAGTCGTTGTTTTGGGGCGAAACAA
CCAAAGTCGGCACGGTTAGATTTAGATCTGTTTTAACGACCGGAAAGCGAGGATTTACCTTGGGATGGT
AGTAACGTAACCCGACGGTATATTTCCCTTTAGGTAAGAAAATTTTGAGTTCTGTTTGGTTGGGGTCAT
CGGTGAGTTGCGTGAAATTTAACACCGTGCGATAGCCCGGAAAGTCATAGATGCAACCGACCCAAGCTT
CCGTGGATTGACGAATCGTTTCGAGGTTAATGGTGAGTTCTTTTTCTACAGAGAGTGGTCCCAGGGTGC
CGATAATGGCGTGGGTATTCCACCGGGGCGCTTTGGTCATTAAAACGGGTAAACTCAGGAATTTCTGGA
GGGATTTTTCTCCAATAATGCGCCATTCCTTAGCATTTTTTTTATTCAGCGCTAAATACAAAGTGTAGA
GATTCCCAATCAGAAATTTGTTGACTTTATAAAAGCCAAAGGAAAAAATCGCCAAGGGAAGTTCTAACC
AGGGTTTGCGCATATGATCAGGAGGAGTCTTTTTTGAGTGCTAGCTCCCCTGACGCAGGGTCACTCTTG
TAAGTTCCAGTAGCACTCTTTTGGCAAGCATTGAAGCATTCAAACCAGTGAAATCCCCTCGCTGGAGCA
GCGAAGTTTAAGCTATCGTTGAAGTAGCCACCTTGGTTAATTAATTGGCGCGCCGAGCATCTCTTCGAA
GTATTCCAGGCATCAAATAAAACGAAAGGCTCAGTCGAAAGACTGGGCCTTTCGTTTTATCTGTTGTTT
GTCGGTGAACGCTCTCTACTAGAGTCACACTGGCTCACCTTCGGGTGGGCCTTTCTGCGTTTATAAAGC
TTTAGTACAAAAAGACGATTAACCCCATGGGTAAAAGCAGGGGAGCCACTAAAGTTCACAGGTTTACAC
CGAATTTTCCATTTGAAAAGTAGTAAATCATACAGAAAACAATCATGTAAAAATTGAATACTCTAATGG
TTTGATGTCCGAAAAAGTCTAGTTTCTTCTATTCTTCGACCAAATCTATGGCAGGGCACTATCACAGAG
CTGGCTTAATAATTTGGGAGAAATGGGTGGGGGCGGACTTTCGTAGAACAATGTAGATTAAAGTACTGT
ACATATGGCAAGCTGGTCCCACCCGCAATTCGAGAAAGAAGTACATCACCATCACCATCATGGCGCAGT
GGGCCAGTTTGCGAACTTTGTAGACCTGTTGCAATACCGTGCCAAGCTGCAAGCACGTAAGACCGTCTT
TAGCTTCCTGGCGGACGGCGAAGCGGAGAGCGCCGCTCTGACCTATGGTGAGCTGGATCAAAAGGCGCA
GGCAATCGCGGCGTTCCTGCAAGCAAATCAGGCACAAGGCCAACGTGCATTGCTGCTGTATCCGCCAGG
TCTGGAGTTCATCGGTGCCTTCCTGGGTTGTCTGTATGCGGGTGTCGTCGCGGTTCCGGCATATCCTCC
GCGTCCGAACAAGTCCTTCGACCGTTTGCACTCCATCATTCAGGACGCCCAAGCGAAGTTTGCACTGAC
GACGACCGAGTTGAAGGATAAGATTGCAGACCGTCTGGAAGCGCTGGAGGGTACGGACTTCCATTGCCT
GGCGACCGACCAAGTCGAGCTGATCAGCGGCAAAAACTGGCAAAAGCCGAATATCTCCGGTACGGATCT
GGCGTTTCTGCAATACACCAGCGGCAGCACGGGTGATCCAAAAGGCGTGATGGTCAGCCACCATAACCT
GATTCACAATAGCGGTCTGATTAACCAGGGTTTCCAAGACACCGAAGCGAGCATGGGTGTGTCCTGGCT
GCCGCCGTATCACGACATGGGTCTGATTGGCGGCATCCTGCAACCTATCTACGTTGGCGCAACGCAAAT
CCTGATGCCACCAGTCGCCTTTCTGCAACGTCCGTTCCGCTGGCTGAAGGCGATCAACGATTACCGTGT
CAGCACCAGCGGTGCGCCGAACTTTGCTTACGACCTGTGCGCTTCTCAGATTACCCCGGAACAAATCCG
CGAGCTGGATCTGAGCTGTTGGCGTCTGGCATTCAGCGGTGCAGAGCCGATTCGCGCTGTCACGCTGGA
AAACTTTGCGAAAACGTTCGCAACCGCGGGTTTCCAGAAATCGGCCTTCTACCCTTGTTACGGTATGGC
GGAAACCACCCTGATCGTGAGCGGTGGCAATGGCCGTGCCCAACTGCCACAGGAGATCATCGTTAGCAA
GCAGGGCATTGAGGCGAACCAAGTGCGTCCGGCTCAAGGCACGGAAACGACCGTGACCCTGGTGGGTAG
CGGTGAGGTCATTGGTGACCAGATCGTTAAGATCGTTGACCCTCAAGCGCTGACCGAGTGCACCGTCGG
TGAAATTGGCGAGGTGTGGGTTAAAGGTGAAAGCGTTGCTCAGGGCTACTGGCAGAAGCCGGACTTGAC
GCAGCAGCAGTTCCAGGGTAACGTGGGTGCCGAAACGGGTTTCCTGCGCACCGGCGATCTGGGTTTCCT
GCAAGGCGGCGAGCTGTATATCACCGGCCGTCTGAAGGATCTGCTGATCATTCGTGGCCGTAATCACTA
TCCTCAGGACATTGAGCTGACCGTGGAAGTTGCTCACCCAGCCCTGCGTCAGGGCGCAGGTGCCGCGGT
GAGCGTGGACGTTAATGGTGAAGAACAACTGGTGATCGTTCAAGAGGTTGAGCGTAAGTACGCACGCAA
GCTGAATGTGGCAGCAGTCGCTCAGGCCATCCGTGGTGCGATTGCGGCAGAGCACCAGTTGCAGCCGCA
GGCGATCTGCTTTATCAAACCGGGCAGCATCCCGAAAACTAGCAGCGGCAAAATCCGTCGTCACGCATG
TAAGGCCGGTTTTCTGGACGGAAGCTTGGCGGTTGTTGGTGAGTGGCAACCGAGCCATCAGAAAGAGGG
CAAAGGTATTGGTACCCAGGCAGTGACCCCGAGCACCACGACGTCCACCAACTTTCCGCTGCCGGATCA
ACACCAGCAACAGATCGAGGCGTGGCTGAAGGACAACATCGCGCACCGCCTGGGTATTACGCCGCAGCA
GTTGGATGAAACGGAACCGTTCGCTTCTTACGGTCTGGACAGCGTTCAAGCAGTCCAGGTCACCGCAGA
CCTGGAGGACTGGCTGGGCCGCAAGCTGGACCCGACTCTGGCCTATGATTACCCGACCATTCGCACGCT
GGCGCAATTCCTGGTTCAGGGCAACCAGGCCTTGGAGAAAATCCCGCAAGTTCCAAAGATTCAGGGTAA
AGAGATTGCGGTGGTGGGCCTGAGCTGCCGCTTTCCGCAGGCGGACAATCCGGAGGCGTTCTGGGAACT
GTTGCGCAATGGCAAGGATGGCGTGCGTCCGCTGAAAACCCGTTGGGCCACTGGTGAGTGGGGTGGTTT
CCTGGAGGATATCGACCAGTTTGAGCCGCAGTTCTTTGGTATTAGCCCGCGTGAGGCGGAGCAAATGGA
CCCGCAACAGCGTCTGCTGCTGGAGGTCACCTGGGAGGCACTGGAGCGTGCGAATATCCCTGCCGAATC
CCTGCGTCACAGCCAGACCGGCGTCTTTGTGGGCATTAGCAACAGCGATTACGCACAACTGCAAGTGCG
TGAGAACAACCCGATCAATCCGTACATGGGTACTGGTAACGCACATAGCATCGCGGCGAATCGTCTGAG
CTACTTTCTGGATCTGCGCGGTGTCTCCCTGAGCATTGATACCGCGTGTTCTAGCAGCCTGGTCGCAGT
TCATCTGGCGTGCCAAAGCCTGATTAACGGCGAGAGCGAGCTGGCGATTGCTGCGGGTGTTAATCTGAT
TCTGACCCCGGATGTCACGCAAACCTTTACCCAAGCGGGTATGATGAGCAAGACGGGCCGTTGCCAGAC
GTTTGATGCGGAGGCGGACGGCTACGTGCGCGGTGAAGGCTGCGGCGTTGTTCTGCTGAAACCGCTGGC
TCAGGCGGAGCGTGATGGCGACAATATCCTGGCGGTCATCCACGGTAGCGCGGTTAACCAGGACGGTCG
CAGCAATGGTCTGACTGCGCCGAACGGCCGCTCTCAGCAAGCGGTTATCCGTCAGGCCCTGGCGCAGGC
GGGCATCACCGCGGCAGACCTGGCGTATTTGGAAGCGCATGGTACGGGCACCCCGCTGGGCGACCCGAT
TGAAATCAACAGCTTGAAAGCAGTGCTGCAAACCGCCCAGCGCGAGCAACCGTGCGTTGTGGGCAGCGT
CAAGACGAACATTGGCCACCTGGAGGCAGCAGCGGGTATTGCAGGTCTGATCAAGGTGATTCTGTCCCT
GGAGCACGGCATGATTCCGCAACACCTGCACTTTAAGCAACTGAATCCGCGCATCGACCTGGACGGCCT
GGTTACCATCGCGAGCAAAGACCAGCCGTGGTCGGGTGGTAGCCAGAAGCGTTTCGCCGGTGTCAGCAG
CTTTGGTTTTGGCGGTACGAATGCTCACGTGATTGTTGGTGATTATGCCCAGCAAAAGTCCCCGCTGGC
TCCGCCTGCGACCCAAGACCGTCCTTGGCATCTGCTGACTCTGAGCGCGAAGAACGCACAAGCGTTGAA
CGCGTTGCAAAAGAGCTATGGTGACTACCTGGCGCAACATCCGAGCGTTGACCCTCGCGATCTGTGCCT
GAGCGCTAACACTGGTCGCTCTCCGCTGAAAGAACGCCGCTTCTTCGTGTTCAAGCAGGTTGCCGACTT
GCAACAAACCCTGAATCAGGACTTTCTGGCGCAGCCGAGGCTGAGCAGCCCAGCCAAGATTGCGTTCCT
GTTCACGGGTCAGGGCAGCCAGTACTACGGTATGGGCCAGCAACTGTATCAGACGTCCCCGGTTTTCCG
TCAAGTCCTGGATGAATGCGACCGTCTGTGGCAGACGTACAGCCCGGAGGCACCGGCGCTGACCGATCT
GCTGTACGGCAATCATAATCCTGACCTGGTTCATGAAACGGTTTACACGCAACCGCTGCTGTTCGCGGT
GGAGTATGCTATCGCGCAGTTGTGGTTGAGCTGGGGCGTTACTCCGGATTTCTGCATGGGTCATAGCGT
CGGTGAGTATGTGGCGGCCTGCCTGGCGGGTGTGTTTAGCCTGGCGGATGGCATGAAACTGATTACCGC
GCGTGGTAAACTGATGCATGCACTGCCGAGCAATGGCAGCATGGCGGCTGTGTTTGCGGACAAAACCGT
TATCAAGCCGTATCTGAGCGAACACCTGACCGTCGGCGCAGAAAATGGCAGCCACCTGGTTCTGAGCGG
TAAGACCCCTTGTCTGGAAGCATCCATCCACAAACTGCAAAGCCAGGGCATCAAAACCAAGCCTCTGAA
AGTCTCCCATGCGTTCCACTCGCCGCTGATGGCGCCGATGCTGGCGGAATTTCGTGAGATCGCCGAACA
GATTACGTTCCATCCGCCACGTATCCCGCTGATTAGCAACGTGACGGGTGGTCAAATCGAGGCCGAGAT
CGCGCAAGCAGACTATTGGGTTAAACATGTTAGCCAGCCGGTGAAGTTCGTTCAGAGCATTCAGACCCT
GGCCCAAGCGGGTGTGAATGTGTACCTGGAAATCGGTGTTAAACCAGTCCTGCTGTCTATGGGTCGCCA
CTGTCTGGCAGAGCAGGAAGCGGTTTGGCTGCCGAGCCTGCGTCCACATAGCGAGCCTTGGCCGGAAAT
CTTGACTAGTCTGGGCAAACTGTACGAGCAAGGTCTGAATATCGACTGGCAAACGGTTGAAGCCGGTGA
TCGCCGTCGTAAGCTGATTTTGCCGACCTACCCGTTCCAGCGTCAGCGTTATTGGTTCAACCAAGGTAG
CTGGCAAACCGTCGAAACTGAGAGCGTGAATCCAGGCCCGGACGACCTGAATGACTGGCTGTACCAAGT
GGCATGGACTCCGCTGGATACGCTGCCGCCTGCACCGGAACCGTCGGCGAAACTGTGGCTGATTCTGGG
TGATCGTCACGATCACCAACCGATTGAGGCCCAGTTCAAAAACGCCCAACGTGTGTACCTGGGCCAAAG
CAACCACTTTCCGACGAACGCCCCGTGGGAGGTGAGCGCGGACGCACTGGATAACTTGTTTACCCATGT
GGGTAGCCAAAACCTGGCAGGCATTCTGTATCTGTGCCCGCCTGGTGAAGATCCGGAGGATCTGGATGA
GATTCAGAAACAAACTTCCGGCTTTGCGTTGCAACTGATTCAGACCCTGTATCAGCAGAAAATCGCAGT
GCCGTGTTGGTTTGTTACCCATCAAAGCCAGCGTGTGCTGGAAACGGACGCGGTGACGGGTTTTGCCCA
AGGTGGTCTGTGGGGTTTGGCGCAAGCGATTGCACTGGAACATCCGGAACTGTGGGGTGGTATCATTGA
CGTGGATGATAGCCTGCCGAACTTCGCGCAGATTTGTCAGCAACGTCAGGTTCAGCAACTGGCTGTCCG
TCACCAGAAACTGTATGGTGCGCAACTGAAGAAGCAGCCGAGCCTGCCGCAGAAGAATCTGCAGATCCA
ACCTCAACAGACCTACCTGGTCACGGGCGGTTTGGGTGCAATCGGTCGTAAGATTGCGCAGTGGCTGGC
GGCTGCGGGTGCTGAGAAAGTTATCCTGGTTAGCCGTCGTGCACCGGCAGCGGATCAACAAACCTTGCC
GACCAACGCCGTGGTGTACCCGTGCGATCTGGCGGATGCGGCGCAGGTTGCGAAACTGTTCCAAACCTA
TCCGCACATTAAGGGTATCTTTCATGCAGCCGGTACGCTGGCTGACGGTTTGCTGCAACAGCAAACCTG
GCAGAAATTCCAGACTGTCGCTGCGGCGAAGATGAAGGGCACCTGGCACCTGCATCGCCACTCTCAGAA
GTTGGACTTGGATTTCTTTGTTTTGTTTTCGTCTGTTGCGGGTGTGCTGGGTAGCCCTGGTCAAGGCAA
TTACGCGGCAGCCAACCGTGGCATGGCCGCCATCGCTCAGTACCGCCAGGCTCAAGGTCTGCCGGCACT
GGCGATTCACTGGGGCCCTTGGGCGGAAGGTGGTATGGCAAACAGCTTGAGCAACCAAAATCTGGCATG
GTTGCCTCCGCCGCAGGGCTTGACCATTCTGGAAAAAGTTTTGGGTGCCCAAGGCGAAATGGGCGTGTT
CAAACCGGACTGGCAGAACTTGGCCAAACAATTCCCGGAGTTCGCGAAAACCCATTACTTTGCGGCGGT
CATTCCGAGCGCTGAAGCGGTTCCACCGACCGCATCTATCTTCGACAAGCTGATCAATCTGGAAGCGAG
CCAGCGCGCAGATTACCTGCTGGACTATCTGCGTAGATCTGTGGCACAAATTCTGAAACTGGAAATTGA
GCAGATTCAGAGCCACGACTCCCTGCTGGATCTGGGTATGGATAGCCTGATGATCATGGAGGCGATTGC
GTCCCTGAAACAAGACCTGCAACTGATGCTGTATCCGCGTGAGATTTACGAGCGTCCGCGTCTGGATGT
TCTGACTGCTTACTTGGCCGCTGAGTTTACCAAAGCGCATGATTCTGAAGCAGCTACCGCCGCAGCTGC
GATCCCTAGCCAGAGCCTGAGCGTCAAAACCAAAAAGCAATGGCAGAAACCGGATCATAAGAACCCGAA
TCCGATTGCGTTCATCCTGAGCAGCCCGCGTAGCGGTAGCACCCTGCTGCGCGTGATGCTGGCCGGTCA
CCCGGGTCTGTATTCCCCACCGGAACTGCACCTGCTGCCGTTTGAAACGATGGGTGACCGCCACCAGGA
ACTGGGTCTGTCTCATCTGGGCGAGGGTCTGCAACGTGCCCTGATGGACTTGGAAAATCTGACGCCGGA
AGCATCCCAGGCAAAGGTGAACCAATGGGTGAAGGCGAATACGCCGATTGCAGACATCTACGCATACCT
GCAACGTCAAGCCGAGCAACGTCTGCTGATTGACAAAAGCCCGAGCTATGGCAGCGACCGCCACATTCT
GGATCACAGCGAGATCCTGTTCGATCAGGCGAAATACATCCACCTGGTTCGCCATCCTTATGCGGTCAT
TGAGAGCTTTACCCGCCTGCGTATGGACAAGCTGCTGGGTGCAGAGCAACAGAATCCGTATGCGCTGGC
GGAAAGCATTTGGCGTACCTCGAATCGCAACATTCTGGACTTGGGTCGTACCGTCGGCGCTGACCGCTA
CCTGCAAGTCATCTACGAGGATCTGGTGCGTGACCCGCGTAAAGTTCTGACCAACATTTGTGATTTTCT
GGGTGTCGATTTCGACGAGGCACTGCTGAATCCGTACTCCGGCGACCGCCTGACCGACGGCCTGCACCA
GCAAAGCATGGGTGTGGGTGACCCGAACTTCTTGCAGCACAAGACCATTGATCCGGCGCTAGCGGACAA
ATGGCGTAGCATTACCCTGCCGGCTGCTCTGCAACTGGATACGATTCAACTGGCCGAAACCTTCGCATA
CGACCTGCCGCAGGAGCCGCAGTTGACGCCGCAGACCCAATCTTTGCCATCGATGGTCGAACGTTTCGT
CACGGTTCGCGGCCTGGAAACCTGTCTGTGCGAGTGGGGTGATCGCCATCAACCTCTGGTCTTGCTGTT
GCACGGTATCCTGGAGCAAGGCGCGTCTTGGCAGTTGATCGCGCCTCAACTGGCAGCGCAGGGCTATTG
GGTCGTCGCTCCGGATCTGCGCGGTCACGGTAAATCTGCGCACGCGCAGTCTTATAGCATGCTGGATTT
TCTGGCCGATGTGGACGCGCTGGCCAAACAGTTGGGCGACCGTCCGTTCACCTTGGTTGGTCACAGCAT
GGGTTCCATCATTGGCGCAATGTATGCTGGCATTCGTCAAACCCAGGTTGAAAAACTGATTCTGGTCGA
AACCATCGTCCCGAATGATATTGATGATGCCGAAACCGGCAATCACCTGACCACCCATCTGGATTACCT
GGCAGCCCCTCCGCAGCACCCGATCTTTCCGAGCCTGGAAGTTGCGGCTCGTCGTCTGCGCCAAGCCAC
CCCGCAGTTGCCGAAAGACCTGTCTGCATTTCTGACGCAACGTTCCACGAAGAGCGTCGAGAAGGGTGT
GCAGTGGCGCTGGGATGCCTTCTTGCGCACCCGTGCAGGTATCGAGTTTAACGGTATCAGCCGTCGCCG
TTATCTGGCGCTGCTGAAAGATATCCAGGCCCCAATTACTTTGATTTACGGTGATCAGTCTGAGTTCAA
TCGCCCAGCAGACCTGCAAGCGATCCAGGCGGCACTGCCGCAAGCGCAACGCCTGACGGTTGCTGGCGG
TCACAACTTGCACTTTGAGAATCCGCAGGCCATCGCCCAGATTGTCTATCAGCAGTTGCAGACACCGGT
TCCGAAAACCCAAGGTTTGCACCATCACCACCATCATAGCGCCTGGAGCCACCCGCAGTTTGAAAAGTA
AGGATCCCTCTATATCAGAATTCGGTTTTCCGTCCTGTCTTGATTTTCAAGCAAACAATGCCTCCGATT
TCTAATCGGAGGCATTTGTTTTTGTTTATTGCAAAAACAAAAAATATTGTTACAAATTTTTACAGGCTA
TTAAGCCTACCGTCATAAATAATTTGCCATTTACTAGTTTTTAATTAACCAGAACCTTGACCGAACGCA
GCGGTGGTAACGGCGCAGTGGCGGTTTTCATGGCTTGTTATGACTGTTTTTTTGGGGTACAGTCTATGC
CTCGGGCATCCAAGCAGCAAGCGCGTTACGCCGTGGGTCGATGTTTGATGTTATGGAGCAGCAACGATG
TTACGCAGCAGGGCAGTCGCCCTAAAACAAAGTTAAACATCATGAGGGAAGCGGTGATCGCCGAAGTAT
CGACTCAACTATCAGAGGTAGTTGGCGTCATCGAGCGCCATCTCGAACCGACGTTGCTGGCCGTACATT
TGTACGGCTCCGCAGTGGATGGCGGCCTGAAGCCACACAGTGATATTGATTTGCTGGTTACGGTGACCG
TAAGGCTTGATGAAACAACGCGGCGAGCTTTGATCAACGACCTTTTGGAAACTTCGGCTTCCCCTGGAG
AGAGCGAGATTCTCCGCGCTGTAGAAGTCACCATTGTTGTGCACGACGACATCATTCCGTGGCGTTATC
CAGCTAAGCGCGAACTGCAATTTGGAGAATGGCAGCGCAATGACATTCTTGCAGGTATCTTCGAGCCAG
CCACGATCGACATTGATCTGGCTATCTTGCTGACAAAAGCAAGAGAACATAGCGTTGCCTTGGTAGGTC
CAGCGGCGGAGGAACTCTTTGATCCGGTTCCTGAACAGGATCTATTTGAGGCGCTAAATGAAACCTTAA
CGCTATGGAACTCGCCGCCCGACTGGGCTGGCGATGAGCGAAATGTAGTGCTTACGTTGTCCCGCATTT
GGTACAGCGCAGTAACCGGCAAAATCGCGCCGAAGGATGTCGCTGCCGACTGGGCAATGGAGCGCCTGC
CGGCCCAGTATCAGCCCGTCATACTTGAAGCTAGACAGGCTTATCTTGGACAAGAAGAAGATCGCTTGG
CCTCGCGCGCAGATCAGTTGGAAGAATTTGTCCACTACGTGAAAGGCGAGATCACCAAGGTAGTCGGCA
AATAATGTCTAACAATTCGTTCAAGCCGACGCCGCTTCGCGGCGCGGCTTAACTCAAGCGTTAGATGCA
CTAAGCACATAATTGCTCACAGCCAAACTATCAGGTCAAGTCTGCTTTTATTATTTTTAAGCGTGCATA
ATAAGCCCTACACAAATTGGGAGATATATCATGAGGCGCGCCTGATCAGTTGGTGCTGCATTAGCTAAG
AAGGTCAGGAGATATTATTCGACATCTAGCTGACGGCCATTGCGATCATAAACGAGGATATCCCACTGG
CCATTTTCAGCGGCTTCAAAGGCAATTTTAGACCCATCAGCACTAATGGTTGGATTACGCACTTCTTGG
TTTAAGTTATCGGTTAAATTCCGCTTTTGTTCAAACTCGCGATCATAGAGATAAATATCAGATTCGCCG
CGACGATTGACCGCAAAGACAATGTAGCGACCATCTTCAGAAACGGCAGGATGGGAGGCAATTTCATTT
AGGGTATTGAGGCCCGGTAACAGAATCGTTTGCCTGGTGCTGGTATCAAATAGATAGATATCCTGGGAA
CCATTGCGGTCTGAGGCAAAAACGAGGTAGGGTTCGGCGATCGCCGGGTCAAATTCGAGGGCCCGACTA
TTTAAACTGCGGCCACCGGGATCAACGGGAAAATTGACAATGCGCGGATAACCAACGCAGCTCTGGAGC
AGCAAACCGAGGCTACCGAGGAAAAAACTGCGTAGAAAAGAAACATAGCGCATAGGTCAAAGGGAAATC
AAAGGGCGGGCGATCGCCAATTTTTCTATAATATTGTCCTAACAGCACACTAAAACAGAGCCATGCTAG
CAAAAATTTGGAGTGCCACCATTGTCGGGGTCGATGCCCTCAGGGTCGGGGTGGAAGTGGATATTTCCG
GCGGCTTACCGAAAATGATGGTGGTCGGACTGCGGCCGGCCAAAATGAAGTGAAGTTCCTATACTTTCT
AGAGAATAGGAACTTCTATAGTGAGTCGAATAAGGGCGACACAAAATTTATTCTAAATGCATAATAAAT
ACTGATAACATCTTATAGTTTGTATTATATTTTGTATTATCGTTGACATGTATAATTTTGATATCAAAA
ACTGATTTTCCCTTTATTATTTTCGAGATTTATTTTCTTAATTCTCTTTAACAAACTAGAAATATTGTA
TATACAAAAAATCATAAATAATAGATGAATAGTTTAATTATAGGTGTTCATCAATCGAAAAAGCAACGT
ATCTTATTTAAAGTGCGTTGCTTTTTTCTCATTTATAAGGTTAAATAATTCTCATATATCAAGCAAAGT
GACAGGCGCCCTTAAATATTCTGACAAATGCTCTTTCCCTAAACTCCCCCCATAAAAAAACCCGCCGAA
GCGGGTTTTTACGTTATTTGCGGATTAACGATTACTCGTTATCAGAACCGCCCAGGGGGCCCGAGCTTA
AGACTGGCCGTCGTTTTACAACACAGAAAGAGTTTGTAGAAACGCAAAAAGGCCATCCGTCAGGGGCCT
TCTGCTTAGTTTGATGCCTGGCAGTTCCCTACTCTCGCCTTCCGCTTCCTCGCTCACTGACTCGCTGCG
CTCGGTCGTTCGGCTGCGGCGAGCGGTATCAGCTCACTCAAAGGCGGTAATACGGTTATCCACAGAATC
AGGGGATAACGCAGGAAAGAACATGTGAGCAAAAGGCCAGCAAAAGGCCAGGAACCGTAAAAAGGCCGC
GTTGCTGGCGTTTTTCCATAGGCTCCGCCCCCCTGACGAGCATCACAAAAATCGACGCTCAAGTCAGAG
GTGGCGAAACCCGACAGGACTATAAAGATACCAGGCGTTTCCCCCTGGAAGCTCCCTCGTGCGCTCTCC
TGTTCCGACCCTGCCGCTTACCGGATACCTGTCCGCCTTTCTCCCTTCGGGAAGCGTGGCGCTTTCTCA
TAGCTCACGCTGTAGGTATCTCAGTTCGGTGTAGGTCGTTCGCTCCAAGCTGGGCTGTGTGCACGAACC
CCCCGTTCAGCCCGACCGCTGCGCCTTATCCGGTAACTATCGTCTTGAGTCCAACCCGGTAAGACACGA
CTTATCGCCACTGGCAGCAGCCACTGGTAACAGGATTAGCAGAGCGAGGTATGTAGGCGGTGCTACAGA
GTTCTTGAAGTGGTGGGCTAACTACGGCTACACTAGAAGAACAGTATTTGGTATCTGCGCTCTGCTGAA
GCCAGTTACCTTCGGAAAAAGAGTTGGTAGCTCTTGATCCGGCAAACAAACCACCGCTGGTAGCGGTGG
TTTTTTTGTTTGCAAGCAGCAGATTACGCGCAGAAAAAAAGGATCTCAAGAAGATCCTTTGATCTTTTC
TACGGGGTCTGACGCTCAGTGGAACGACGCGCGCGTAACTCACGTTAAGGGATTTTGGTCATGAGCTTG
CGCCGTCCCGTCAAGTCAGCGTAATGCTCTGCTT

What is claimed is:

1. A method for the biosynthetic production of 1-alkenes, comprising culturing an engineered microorganism in a culture medium, wherein said engineered microorganism comprises a recombinant alpha-olefin-associated enzyme, wherein said engineered microorganism produces 1-alkenes, and wherein the amount of said 1-alkenes produced by said engineered microorganism is greater than the amount that would be produced by an otherwise identical microorganism, cultured under identical conditions, but lacking said recombinant alpha-olefin-associated enzyme.

2. The method of claim 1, wherein said engineered microorganism is a cyanobacterium.

3. The method of claim 1, wherein said cyanobacterium is a Synechococcus species.

4. The method of claim 1, wherein said engineered microorganism comprises a recombinant 1-alkene synthase.

5. The method of claim 4, wherein said recombinant 1-alkene synthase is at least 90% identical to YP_{—}001734428 from Synechococcus sp. PCC 7002.

6. The method of claim 4, wherein said recombinant 1-alkene synthase is at least 90% identical to SEQ ID NO: 5.

7. The method of claim 4, wherein said recombinant 1-alkene synthase is encoded by a gene at least 90% identical to a nucleotide sequence selected from the group consisting of: SEQ ID NO: 2 and SEQ ID NO: 4.

8. The method of claim 1, wherein said recombinant alpha-olefin-associated enzyme is at least 90% identical to YP_{—}0001735499 from Synechococcus sp. PCC 7002.

9. The method of claim 1, wherein said recombinant alpha-olefin enzyme is at least 90% identical to SEQ ID NO: 7.

10. The method of claim 1, wherein said recombinant alpha-olefin enzyme is encoded by a gene at least 90% identical to SEQ ID NO: 6.

11. The method of claim 1, wherein said recombinant alpha-olefin-associated enzyme is at least 90% identical to an amino acid sequence selected from the group consisting of: YP_{—}0001735499 from Synechococcus sp. PCC 7002; YP_{—}003887108.1 from Cyanothece sp. PCC 7822; YP_{—}002377175 from Cyanothece sp. PCC 7424; ZP_{—}08425909.1 from Lyngbya majuscule 3L; ZP_{—}08432358 from Lyngbya majuscule 3L; and YP_{—}003265309 from Haliangium ochraceum DSM 14365.

12. The method of claim 1, wherein said recombinant alpha-olefin-associated enzyme is at least 90% identical to an amino acid sequence selected from the group consisting of: SEQ ID NO:7, SEQ ID NO:9, SEQ ID NO:11, SEQ ID NO:13, SEQ ID NO:15, SEQ ID NO:17, and SEQ ID NO: 19.

13. The method of claim 1, wherein said recombinant alpha-olefin-associated enzyme is encoded by a gene at least 90% identical to a nucleotide sequence selected from the group consisting of: SEQ ID NO:6, SEQ ID NO:8, SEQ ID NO:10, SEQ ID NO:12, SEQ ID NO:14, SEQ ID NO:16, and SEQ ID NO: 18.

14. The method of any of claims 1-13, wherein said recombinant alpha-olefin-associated enzyme is an endogenous alpha-olefin-associated enzyme expressed by a gene operably linked to a promoter other than its native promoter.

15. The method of any of claims 1-13, wherein said recombinant alpha-olefin-associated enzyme is a heterologous alpha-olefin-associated enzyme.

16. The method of any of claims 1-13, wherein said recombinant alpha-olefin-associated enzyme is expressed from a heterologous promoter.

17. The method of claim 16, wherein said promoter is tsr2142.

18. The method of claim 16, wherein said promoter is at least 90% identical to SEQ ID NO: 20.

19. The method of claim 16 wherein said alpha-olefin-associated enzyme is endogenous to said microorganism.

20. The method of any of claims 1 and 4-13, wherein said engineered microorganism is a photosynthetic microorganism, and wherein exposing said engineered microorganism to light and an inorganic carbon source results in the production of alkenes by said microorganism.

21. The method of any of claims 1 and 4-13, wherein said engineered microorganism is a cyanobacterium.

22. The method claim 21, wherein said engineered cyanobacterium is an engineered Synechococcus species.

23. The method of any of claims 1-13, wherein said 1-alkenes are selected from the group consisting of: 1-tridecene, 1-tetradecene, 1-pentadecene, 1-hexadecene, 1-heptadecene, 1-octadecene, 1-nonadecene and 1-octadecene, and 1,x-nonadecadiene.

24. The method of claim 23, wherein said 1,x-nonadecadiene is 1,12-(cis)-nonadecadiene.

25. The method of any of claims 1-13, further comprising isolating said 1-alkenes from said cyanobacterium or said culture medium.

26. The method of any of claims 1-13, wherein the amount of said 1-alkenes produced by said engineered microorganism is at least four times greater than the amount that would be produced by an otherwise identical microorganism, cultured under identical conditions, but lacking said recombinant alpha-olefin associated enzyme.

27. The method of any of claims 1-13, wherein the rate of production of said 1-alkenes by said engineered microorganism is greater than 0.05, 0.06, 0.07, 0.08, 0.09, 0.10, 0.11, 0.12, 0.13, 0.14, 0.15, 0.16, 0.17, or 0.18 mg*L⁻¹*h⁻¹.

28. The method of any of claims 1-13, wherein said production of 1-alkenes is inhibited by the presence of 15 μM urea in said culture medium.

29. An isolated or recombinant polynucleotide comprising or consisting of a nucleic acid sequence selected from the group consisting of:
a. SEQ ID NO:6, SEQ ID NO:8, SEQ ID NO:10, SEQ ID NO:12, SEQ ID NO:14, SEQ ID NO: 16, or SEQ ID NO:18;
b. a nucleic acid sequence that is a degenerate variant of SEQ ID NO:6, SEQ ID NO:8, SEQ ID NO:10, SEQ ID NO:12, SEQ ID NO:14, SEQ ID NO: 16, or SEQ ID NO:18;
c. a nucleic acid sequence at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 90%, at least 95%, at least 98%, at least 99% or at least 99.9% identical to SEQ ID NO:6, SEQ ID NO:8, SEQ ID NO:10, SEQ ID NO:12, SEQ ID NO:14, SEQ ID NO: 16, or SEQ ID NO:18;
d. a nucleic acid sequence that encodes a polypeptide having the amino acid sequence of SEQ ID NO:7, SEQ ID NO:9, SEQ ID NO:11, SEQ ID NO:13, SEQ ID NO:15, SEQ ID NO: 17, or SEQ ID NO:19;
e. a nucleic acid sequence that encodes a polypeptide at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%%, at least 99.1%, at least 99.2%, at least 99.3%, at least 99.4%, at least 99.5%, at least 99.6%, at least 99.7%, at least 99.8% or at least 99.9% identical to SEQ ID NO:7, SEQ ID NO:9, SEQ ID NO:11, SEQ ID NO:13, SEQ ID NO:15, SEQ ID NO: 17, or SEQ ID NO:19; and
f. a nucleic acid sequence that hybridizes under stringent conditions to SEQ ID NO:6, SEQ ID NO:8, SEQ ID NO:10, SEQ ID NO:12, SEQ ID NO:14, SEQ ID NO: 16, or SEQ ID NO:18.

30. The isolated or recombinant polynucleotide of claim 29, wherein the nucleic acid sequence encodes a polypeptide having alpha-olefin synthesis-associated activity.

31. The isolated or recombinant polynucleotide of claim 29 or 30, wherein the nucleic acid sequence and the sequence of interest are operably linked to one or more expression control sequences.

32. A vector comprising the isolated polynucleotide of claim 29 or 30.

33. The vector of claim 32, further comprising a nucleotide sequence at least 90% identical to SEQ ID NO: 20.

34. The vector of claim 32, further comprising a nucleotide sequence at least 90% identical to SEQ ID NO: 21.

35. The vector of claim 32, wherein said vector comprises a spectinomycin resistance marker.

36. The vector of claim 35, wherein said spectinomycin resistance marker is encoded by a nucleotide sequence at least 90% identical to SEQ ID NO: 22.

37. The vector of claim 30, wherein said vector is encoded by a nucleotide sequence at least 90% identical to SEQ ID NO: 23.

38. A fusion protein comprising an isolated peptide encoded by an isolated or recombinant polynucleotide of claim 29 or 30 fused to a heterologous amino acid sequence.

39. A host cell comprising the isolated polynucleotide of claim 29 or 30.

40. The host cell of claim 39, wherein the host cell is selected from the group consisting of prokaryotes, eukaryotes, yeasts, filamentous fungi, protozoa, algae and synthetic cells.

41. The host cell of claim 39, wherein said host cell is cyanobacteria.

42. The host cell of claim 41, wherein said cyanobacteria is Synechococcus.

43. The host cell of claim 39 wherein the host cell produces a carbon-based product of interest.

44. The host cell of claim 43, wherein said carbon-based product of interest is 1-alkene.

45. An isolated antibody or antigen-binding fragment or derivative thereof which binds selectively to an isolated peptide encoded by an isolated or recombinant polynucleotide of claim 29 or 30.

46. A method for producing carbon-based products of interest comprising:
a. culturing a recombinant host cell engineered to produce carbon-based products of interest, wherein said host cell comprises the isolated or recombinant nucleotide sequence of claim 29 or 30; and
b. removing the carbon-based product of interest.

47. The method of claim 46 wherein the recombinant nucleotide sequence encodes a polypeptide having alpha-olefin synthesis-associated activity.

48. A method for identifying a modified gene that improves 1-alkene synthesis comprising:
a. identifying a polynucleotide sequence expressing an enzyme involved in 1-alkene biosynthesis;
b. expressing said enzyme from a recombinant form of the polynucleotide sequence in a host cell; and
c. screening the host cell for increased activity of said enzyme or increased production of 1-alkene.


SEQUENCE LISTING (25 sequences)

SEQ ID NO: 1 - DNA, 675 bp, Artificial Sequence
  Description of Artificial Sequence Synthetic polynucleotide
atgaaaatttacggcatttacatggaccgtcctttgagccaagaagaaaatgagcgttttatgtcgttcatcagcccggaaaaacgcgagaagtgccgtcgtttctatcataaggaggatgcccatcgcacgctgctgggtgatgttctggttcgttccgtgatctcccgccaataccagctggacaaaagcgatatccgcttttccacccaggagtacggcaaaccatgtatcccggacctgccggacgctcacttcaacattagccacagcggtcgttgggtgatttgtgcgttcgatagccagccgattggtattgacattgaaaagacgaagcctattagcctggagatcgccaagcgcttcttcagcaaaaccgagtatagcgatctgctggcgaaagacaaagacgagcaaaccgactacttttaccacctgtggagcatgaaagaaagctttatcaagcaagaaggtaagggtttgagcttgccgctggacagctttagcgtgcgtctgcatcaggatggtcaggtcagcatcgagctgccggactctcactctccgtgctatattaaaacctacgaggtcgatccgggctataaaatggcggtttgcgcagcacacccggactttccggaggatatcactatggtgagctatgaagagttgctgtaa

SEQ ID NO: 2 - DNA, 8277 bp, Artificial Sequence
  Description of Artificial Sequence Synthetic polynucleotide
atggcaagctggtcccacccgcaattcgagaaagaagtacatcaccatcaccatcatggcgcagtgggccagtttgcgaactttgtagacctgttgcaataccgtgccaagctgcaagcacgtaagaccgtctttagcttcctggcggacggcgaagcggagagcgccgctctgacctatggtgagctggatcaaaaggcgcaggcaatcgcggcgttcctgcaagcaaatcaggcacaaggccaacgtgcattgctgctgtatccgccaggtctggagttcatcggtgccttcctgggttgtctgtatgcgggtgtcgtcgcggttccggcatatcctccgcgtccgaacaagtccttcgaccgtttgcactccatcattcaggacgcccaagcgaagtttgcactgacgacgaccgagttgaaggataagattgcagaccgtctggaagcgctggagggtacggacttccattgcctggcgaccgaccaagtcgagctgatcagcggcaaaaactggcaaaagccgaatatctccggtacggatctggcgtttctgcaatacaccagcggcagcacgggtgatccaaaaggcgtgatggtcagccaccataacctgattcacaatagcggtctgattaaccagggtttccaagacaccgaagcgagcatgggtgtgtcctggctgccgccgtatcacgacatgggtctgattggcggcatcctgcaacctatctacgttggcgcaacgcaaatcctgatgccaccagtcgcctttctgcaacgtccgttccgctggctgaaggcgatcaacgattaccgtgtcagcaccagcggtgcgccgaactttgcttacgacctgtgcgcttctcagattaccccggaacaaatccgcgagctggatctgagctgttggcgtctggcattcagcggtgcagagccgattcgcgctgtcacgctggaaaactttgcgaaaacgttcgcaaccgcgggtttccagaaatcggccttctacccttgttacggtatggcggaaaccaccctgatcgtgagcggtggcaatggccgtgcccaactgccacaggagatcatcgttagcaagcagggcattgaggcgaaccaagtgcgtccggctcaaggcacggaaacgaccgtgaccctggtgggtagcggtgaggtcattggtgaccagatcgttaagatcgttgaccctcaagcgctgaccgagtgcaccgtcggtgaaattggcgaggtgtgggttaaaggtgaaagcgttgctcagggctactggcagaagccggacttgacgcagcagcagttccagggtaacgtgggtgccgaaacgggtttcctgcgcaccggcgatctgggtttcctgcaaggcggcgagctgtatatcaccggccgtctgaaggatctgctgatcattcgtggccgtaatcactatcctcaggacattgagctgaccgtggaagttgctcacccagccctgcgtcagggcgcaggtgccgcggtgagcgtggacgttaatggtgaagaacaactggtgatcgttcaagaggttgagcgtaagtacgcacgcaagctgaatgtggcagcagtcgctcaggccatccgtggtgcgattgcggcagagcaccagttgcagccgcaggcgatctgctttatcaaaccgggcagcatcccgaaaactagcagcggcaaaatccgtcgtcacgcatgtaaggccggttttctggacggaagcttggcggttgttggtgagtggcaaccgagccatcagaaagagggcaaaggtattggtacccaggcagtgaccccgagcaccacgacgtccaccaactttccgctgccggatcaacaccagcaacagatcgaggcgtggctgaaggacaacatcgcgcaccgcctgggtattacgccgcagcagttggatgaaacggaaccgttcgcttcttacggtctggacagcgttcaagcagtccaggtcaccgcagacctggaggactggctgggccgcaagctggacccgactctggcctatgattacccgaccattcgcacgctggcgcaattcctggttcagggcaaccaggccttggagaaaatcccgcaagttccaaagattcagggtaaagagattgcggtggtgggcctgagctgccgctttccgcaggcggacaatccggaggcgttctgggaactgttgcgcaatggcaaggatggcgtgcgtccgctgaaaacccgttgggccactggtgagtggggtggtttcctggaggatatcgaccagtttgagccgcagttctttggtattagcccgcgtgaggcggagcaaatggacccgcaacagcgtctgctgctggaggtcacctgggaggcactggagcgtgcgaatatccctgccgaatccctgcgtcacagccagaccggcgtctttgtgggcattagcaacagcgattacgcacaactgcaagtgcgtgagaacaacccgatcaatccgtacatgggtactggtaacgcacatagcatcgcggcgaatcgtctgagctactttctggatctgcgcggtgtctccctgagcattgataccgcgtgttctagcagcctggtcgcagttcatctggcgtgccaaagcctgattaacggcgagagcgagctggcgattgctgcgggtgttaatctgattctgaccccggatgtcacgcaaacctttacccaagcgggtatgatgagcaagacgggccgttgccagacgtttgatgcggaggcggacggctacgtgcgcggtgaaggctgcggcgttgttctgctgaaaccgctggctcaggcggagcgtgatggcgacaatatcctggcggtcatccacggtagcgcggttaaccaggacggtcgcagcaatggtctgactgcgccgaacggccgctctcagcaagcggttatccgtcaggccctggcgcaggcgggcatcaccgcggcagacctggcgtatttggaagcgcatggtacgggcaccccgctgggcgacccgattgaaatcaacagcttgaaagcagtgctgcaaaccgcccagcgcgagcaaccgtgcgttgtgggcagcgtcaagacgaacattggccacctggaggcagcagcgggtattgcaggtctgatcaaggtgattctgtccctggagcacggcatgattccgcaacacctgcactttaagcaactgaatccgcgcatcgacctggacggcctggttaccatcgcgagcaaagaccagccgtggtcgggtggtagccagaagcgtttcgccggtgtcagcagctttggttttggcggtacgaatgctcacgtgattgttggtgattatgcccagcaaaagtccccgctggctccgcctgcgacccaagaccgtccttggcatctgctgactctgagcgcgaagaacgcacaagcgttgaacgcgttgcaaaagagctatggtgactacctggcgcaacatccgagcgttgaccctcgcgatctgtgcctgagcgctaacactggtcgctctccgctgaaagaacgccgcttcttcgtgttcaagcaggttgccgacttgcaacaaaccctgaatcaggactttctggcgcagccgaggctgagcagcccagccaagattgcgttcctgttcacgggtcagggcagccagtactacggtatgggccagcaactgtatcagacgtccccggttttccgtcaagtcctggatgaatgcgaccgtctgtggcagacgtacagcccggaggcaccggcgctgaccgatctgctgtacggcaatcataatcctgacctggttcatgaaacggtttacacgcaaccgctgctgttcgcggtggagtatgctatcgcgcagttgtggttgagctggggcgttactccggatttctgcatgggtcatagcgtcggtgagtatgtggcggcctgcctggcgggtgtgtttagcctggcggatggcatgaaactgattaccgcgcgtggtaaactgatgcatgcactgccgagcaatggcagcatggcggctgtgtttgcggacaaaaccgttatcaagccgtatctgagcgaacacctgaccgtcggcgcagaaaatggcagccacctggttctgagcggtaagaccccttgtctggaagcatccatccacaaactgcaaagccagggcatcaaaaccaagcctctgaaagtctcccatgcgttccactcgccgctgatggcgccgatgctggcggaatttcgtgagatcgccgaacagattacgttccatccgccacgtatcccgctgattagcaacgtgacgggtggtcaaatcgaggccgagatcgcgcaagcagactattgggttaaacatgttagccagccggtgaagttcgttcagagcattcagaccctggcccaagcgggtgtgaatgtgtacctggaaatcggtgttaaaccagtcctgctgtctatgggtcgccactgtctggcagagcaggaagcggtttggctgccgagcctgcgtccacatagcgagccttggccggaaatcttgactagtctgggcaaactgtacgagcaaggtctgaatatcgactggcaaacggttgaagccggtgatcgccgtcgtaagctgattttgccgacctacccgttccagcgtcagcgttattggttcaaccaaggtagctggcaaaccgtcgaaactgagagcgtgaatccaggcccggacgacctgaatgactggctgtaccaagtggcatggactccgctggatacgctgccgcctgcaccggaaccgtcggcgaaactgtggctgattctgggtgatcgtcacgatcaccaaccgattgaggcccagttcaaaaacgcccaacgtgtgtacctgggccaaagcaaccactttccgacgaacgccccgtgggaggtgagcgcggacgcactggataacttgtttacccatgtgggtagccaaaacctggcaggcattctgtatctgtgcccgcctggtgaagatccggaggatctggatgagattcagaaacaaacttccggctttgcgttgcaactgattcagaccctgtatcagcagaaaatcgcagtgccgtgttggtttgttacccatcaaagccagcgtgtgctggaaacggacgcggtgacgggttttgcccaaggtggtctgtggggtttggcgcaagcgattgcactggaacatccggaactgtggggtggtatcattgacgtggatgatagcctgccgaacttcgcgcagatttgtcagcaacgtcaggttcagcaactggctgtccgtcaccagaaactgtatggtgcgcaactgaagaagcagccgagcctgccgcagaagaatctgcagatccaacctcaacagacctacctggtcacgggcggtttgggtgcaatcggtcgtaagattgcgcagtggctggcggctgcgggtgctgagaaagttatcctggttagccgtcgtgcaccggcagcggatcaacaaaccttgccgaccaacgccgtggtgtacccgtgcgatctggcggatgcggcgcaggttgcgaaactgttccaaacctatccgcacattaagggtatctttcatgcagccggtacgctggctgacggtttgctgcaacagcaaacctggcagaaattccagactgtcgctgcggcgaagatgaagggcacctggcacctgcatcgccactctcagaagttggacttggatttctttgttttgttttcgtctgttgcgggtgtgctgggtagccctggtcaaggcaattacgcggcagccaaccgtggcatggccgccatcgctcagtaccgccaggctcaaggtctgccggcactggcgattcactggggcccttgggcggaaggtggtatggcaaacagcttgagcaaccaaaatctggcatggttgcctccgccgcagggcttgaccattctggaaaaagttttgggtgcccaaggcgaaatgggcgtgttcaaaccggactggcagaacttggccaaacaattcccggagttcgcgaaaacccattactttgcggcggtcattccgagcgctgaagcggttccaccgaccgcatctatcttcgacaagctgatcaatctggaagcgagccagcgcgcagattacctgctggactatctgcgtagatctgtggcacaaattctgaaactggaaattgagcagattcagagccacgactccctgctggatctgggtatggatagcctgatgatcatggaggcgattgcgtccctgaaacaagacctgcaactgatgctgtatccgcgtgagatttacgagcgtccgcgtctggatgttctgactgcttacttggccgctgagtttaccaaagcgcatgattctgaagcagctaccgccgcagctgcgatccctagccagagcctgagcgtcaaaaccaaaaagcaatggcagaaaccggatcataagaacccgaatccgattgcgttcatcctgagcagcccgcgtagcggtagcaccctgctgcgcgtgatgctggccggtcacccgggtctgtattccccaccggaactgcacctgctgccgtttgaaacgatgggtgaccgccaccaggaactgggtctgtctcatctgggcgagggtctgcaacgtgccctgatggacttggaaaatctgacgccggaagcatcccaggcaaaggtgaaccaatgggtgaaggcgaatacgccgattgcagacatctacgcatacctgcaacgtcaagccgagcaacgtctgctgattgacaaaagcccgagctatggcagcgaccgccacattctggatcacagcgagatcctgttcgatcaggcgaaatacatccacctggttcgccatccttatgcggtcattgagagctttacccgcctgcgtatggacaagctgctgggtgcagagcaacagaatccgtatgcgctggcggaaagcatttggcgtacctcgaatcgcaacattctggacttgggtcgtaccgtcggcgctgaccgctacctgcaagtcatctacgaggatctggtgcgtgacccgcgtaaagttctgaccaacatttgtgattttctgggtgtcgatttcgacgaggcactgctgaatccgtactccggcgaccgcctgaccgacggcctgcaccagcaaagcatgggtgtgggtgacccgaacttcttgcagcacaagaccattgatccggcgctagcggacaaatggcgtagcattaccctgccggctgctctgcaactggatacgattcaactggccgaaaccttcgcatacgacctgccgcaggagccgcagttgacgccgcagacccaatctttgccatcgatggtcgaacgtttcgtcacggttcgcggcctggaaacctgtctgtgcgagtggggtgatcgccatcaacctctggtcttgctgttgcacggtatcctggagcaaggcgcgtcttggcagttgatcgcgcctcaactggcagcgcagggctattgggtcgtcgctccggatctgcgcggtcacggtaaatctgcgcacgcgcagtcttatagcatgctggattttctggccgatgtggacgcgctggccaaacagttgggcgaccgtccgttcaccttggttggtcacagcatgggttccatcattggcgcaatgtatgctggcattcgtcaaacccaggttgaaaaactgattctggtcgaaaccatcgtcccgaatgatattgatgatgccgaaaccggcaatcacctgaccacccatctggattacctggcagcccctccgcagcacccgatctttccgagcctggaagttgcggctcgtcgtctgcgccaagccaccccgcagttgccgaaagacctgtctgcatttctgacgcaacgttccacgaagagcgtcgagaagggtgtgcagtggcgctgggatgccttcttgcgcacccgtgcaggtatcgagtttaacggtatcagccgtcgccgttatctggcgctgctgaaagatatccaggccccaattactttgatttacggtgatcagtctgagttcaatcgcccagcagacctgcaagcgatccaggcggcactgccgcaagcgcaacgcctgacggttgctggcggtcacaacttgcactttgagaatccgcaggccatcgcccagattgtctatcagcagttgcagacaccggttccgaaaacccaaggtttgcaccatcaccaccatcatagcgcctggagccacccgcagtttgaaaagtaa

SEQ ID NO: 3 - protein, 2758 aa, Artificial Sequence
  Description of Artificial Sequence Synthetic polypeptide
MASWSHPQFEKEVHHHHHHGAVGQFANFVDLLQYRAKLQARKTVFSFLADGEAESAALTYGELDQKAQAIAAFLQANQAQGQRALLLYPPGLEFIGAFLGCLYAGVVAVPAYPPRPNKSFDRLHSIIQDAQAKFALTTTELKDKIADRLEALEGTDFHCLATDQVELISGKNWQKPNISGTDLAFLQYTSGSTGDPKGVMVSHHNLIHNSGLINQGFQDTEASMGVSWLPPYHDMGLIGGILQPIYVGATQILMPPVAFLQRPFRWLKAINDYRVSTSGAPNFAYDLCASQITPEQIRELDLSCWRLAFSGAEPIRAVTLENFAKTFATAGFQKSAFYPCYGMAETTLIVSGGNGRAQLPQEIIVSKQGIEANQVRPAQGTETTVTLVGSGEVIGDQIVKIVDPQALTECTVGEIGEVWVKGESVAQGYWQKPDLTQQQFQGNVGAETGFLRTGDLGFLQGGELYITGRLKDLLIIRGRNHYPQDIELTVEVAHPALRQGAGAAVSVDVNGEEQLVIVQEVERKYARKLNVAAVAQAIRGAIAAEHQLQPQAICFIKPGSIPKTSSGKIRRHACKAGFLDGSLAVVGEWQPSHQKEGKGIGTQAVTPSTTTSTNFPLPDQHQQQIEAWLKDNIAHRLGITPQQLDETEPFASYGLDSVQAVQVTADLEDWLGRKLDPTLAYDYPTIRTLAQFLVQGNQALEKIPQVPKIQGKEIAVVGLSCRFPQADNPEAFWELLRNGKDGVRPLKTRWATGEWGGFLEDIDQFEPQFFGISPREAEQMDPQQRLLLEVTWEALERANIPAESLRHSQTGVFVGISNSDYAQLQVRENNPINPYMGTGNAHSIAANRLSYFLDLRGVSLSIDTACSSSLVAVHLACQSLINGESELAIAAGVNLILTPDVTQTFTQAGMMSKTGRCQTFDAEADGYVRGEGCGVVLLKPLAQAERDGDNILAVIHGSAVNQDGRSNGLTAPNGRSQQAVIRQALAQAGITAADLAYLEAHGTGTPLGDPIEINSLKAVLQTAQREQPCVVGSVKTNIGHLEAAAGIAGLIKVILSLEHGMIPQHLHFKQLNPRIDLDGLVTIASKDQPWSGGSQKRFAGVSSFGFGGTNAHVIVGDYAQQKSPLAPPATQDRPWHLLTLSAKNAQALNALQKSYGDYLAQHPSVDPRDLCLSANTGRSPLKERRFFVFKQVADLQQTLNQDFLAQPRLSSPAKIAFLFTGQGSQYYGMGQQLYQTSPVFRQVLDECDRLWQTYSPEAPALTDLLYGNHNPDLVHETVYTQPLLFAVEYAIAQLWLSWGVTPDFCMGHSVGEYVAACLAGVFSLADGMKLITARGKLMHALPSNGSMAAVFADKTVIKPYLSEHLTVGAENGSHLVLSGKTPCLEASIHKLQSQGIKTKPLKVSHAFHSPLMAPMLAEFREIAEQITFHPPRIPLISNVTGGQIEAEIAQADYWVKHVSQPVKFVQSIQTLAQAGVNVYLEIGVKPVLLSMGRHCLAEQEAVWLPSLRPHSEPWPEILTSLGKLYEQGLNIDWQTVEAGDRRRKLILPTYPFQRQRYWFNQGSWQTVETESVNPGPDDLNDWLYQVAWTPLDTLPPAPEPSAKLWLILGDRHDHQPIEAQFKNAQRVYLGQSNHFPTNAPWEVSADALDNLFTHVGSQNLAGILYLCPPGEDPEDLDEIQKQTSGFALQLIQTLYQQKIAVPCWFVTHQSQRVLETDAVTGFAQGGLWGLAQAIALEHPELWGGIIDVDDSLPNFAQICQQRQVQQLAVRHQKLYGAQLKKQPSLPQKNLQIQPQQTYLVTGGLGAIGRKIAQWLAAAGAEKVILVSRRAPAADQQTLPTNAVVYPCDLADAAQVAKLFQTYPHIKGIFHAAGTLADGLLQQQTWQKFQTVAAAKMKGTWHLHRHSQKLDLDFFVLFSSVAGVLGSPGQGNYAAANRGMAAIAQYRQAQGLPALAIHWGPWAEGGMANSLSNQNLAWLPPPQGLTILEKVLGAQGEMGVFKPDWQNLAKQFPEFAKTHYFAAVIPSAEAVPPTASIFDKLINLEASQRADYLLDYLRRSVAQILKLEIEQIQSHDSLLDLGMDSLMIMEAIASLKQDLQLMLYPREIYERPRLDVLTAYLAAEFTKAHDSEAATAAAAIPSQSLSVKTKKQWQKPDHKNPNPIAFILSSPRSGSTLLRVMLAGHPGLYSPPELHLLPFETMGDRHQELGLSHLGEGLQRALMDLENLTPEASQAKVNQWVKANTPIADIYAYLQRQAEQRLLIDKSPSYGSDRHILDHSEILFDQAKYIHLVRHPYAVIESFTRLRMDKLLGAEQQNPYALAESIWRTSNRNILDLGRTVGADRYLQVIYEDLVRDPRKVLTNICDFLGVDFDEALLNPYSGDRLTDGLHQQSMGVGDPNFLQHKTIDPALADKWRSITLPAALQLDTIQLAETFAYDLPQEPQLTPQTQSLPSMVERFVTVRGLETCLCEWGDRHQPLVLLLHGILEQGASWQLIAPQLAAQGYWVVAPDLRGHGKSAHAQSYSMLDFLADVDALAKQLGDRPFTLVGHSMGSIIGAMYAGIRQTQVEKLILVETIVPNDIDDAETGNHLTTHLDYLAAPPQHPIFPSLEVAARRLRQATPQLPKDLSAFLTQRSTKSVEKGVQWRWDAFLRTRAGIEFNGISRRRYLALLKDIQAPITLIYGDQSEFNRPADLQAIQAALPQAQRLTVAGGHNLHFENPQAIAQIVYQQLQTPVPKTQGLHHHHHHSAWSHPQFEK

SEQ ID NO: 4 - DNA, 8163 bp, Synechococcus sp.
atggttggtcaatttgcaaatttcgtcgatctgctccagtacagagctaaacttcaggcgcggaaaaccgtgtttagttttctggctgatggcgaagcggaatctgcggccctgacctacggagaattagaccaaaaagcccaggcgatcgccgcttttttgcaagctaaccaggctcaagggcaacgggcattattactttatccaccgggtttagagtttatcggtgcctttttgggatgtttgtatgctggtgttgttgcggtgccagcttacccaccacggccgaataaatcctttgaccgcctccatagcattatccaagatgcccaggcaaaatttgccctcaccacaacagaacttaaagataaaattgccgatcgcctcgaagctttagaaggtacggattttcattgtttggctacagatcaagttgaattaatttcaggaaaaaattggcaaaaaccgaacatttccggcacagatctcgcttttttgcaatacaccagtggctccacgggcgatcctaaaggagtgatggtttcccaccacaatttgatccacaactccggcttgattaaccaaggattccaggatacagaggcgagtatgggcgtttcctggttgccgccctaccatgatatgggcttgatcggtgggattttacagcccatctatgtgggagcaacgcaaattttaatgcctcccgtggcctttttgcagcgaccttttcggtggctaaaggcgatcaacgattatcgggtttccaccagcggtgcgccgaattttgcctatgatctctgtgccagccaaattaccccggaacaaatcagagaactcgatttgagctgttggcgactggctttttccggggccgaaccgatccgcgctgtgaccctcgaaaattttgcgaaaaccttcgctacagcaggctttcaaaaatcagcattttatccctgttatggtatggctgaaaccaccctgatcgtttccggtggtaatggtcgtgcccagcttccccaggaaattatcgtcagcaaacagggcatcgaagcaaaccaagttcgccctgcccaagggacagaaacaacggtgaccttggtcggcagtggtgaagtgattggcgaccaaattgtcaaaattgttgacccccaggctttaacagaatgtaccgtcggtgaaattggcgaagtatgggttaagggcgaaagtgttgcccagggctattggcaaaagccagacctcacccagcaacaattccagggaaacgtcggtgcagaaacgggctttttacgcacgggcgatctgggttttttgcaaggtggcgaactgtatattacgggtcgtttaaaggatctcctgattatccgggggcgcaaccactatccccaggacattgaattaaccgtcgaagtggcccatcccgctttacgacagggggccggagccgctgtatcagtagacgttaacggggaagaacagttagtcattgtccaggaagttgagcgtaaatatgcccgcaaattaaatgtcgcggcagtagcccaagctattcgtggggcgatcgccgccgaacatcaactgcaaccccaggccatttgttttattaaacccggtagcattcccaaaacatccagcgggaagattcgtcgccatgcctgcaaagctggttttctagacggaagcttggctgtggttggggagtggcaacccagccaccaaaaagaaggaaaaggaattgggacacaagccgttaccccttctacgacaacatcaacgaattttcccctgcctgaccagcaccaacagcaaattgaagcctggcttaaggataatattgcccatcgcctcggcattacgccccaacaattagacgaaacggaaccctttgcaagttatgggctggattcagtgcaagcagtacaggtcacagccgacttagaggattggctaggtcgaaaattagaccccactctggcctacgattatccgaccattcgcaccctggctcagtttttggtccagggtaatcaagcgctagagaaaataccacaggtgccgaaaattcagggcaaagaaattgccgtggtgggtctcagttgtcgttttccccaagctgacaaccccgaagctttttgggaattattacgtaatggtaaagatggagttcgcccccttaaaactcgctgggccacgggagaatggggtggttttttagaagatattgaccagtttgagccgcaattttttggcatttccccccgggaagcggaacaaatggatccccagcaacgcttactgttagaagtaacctgggaagccttggaacgggcaaatattccggcagaaagtttacgccattcccaaacgggggtttttgtcggcattagtaatagtgattatgcccagttgcaggtgcgggaaaacaatccgatcaatccctacatggggacgggcaacgcccacagtattgctgcgaatcgtctgtcttatttcctcgatctccggggcgtttctctgagcatcgatacggcctgttcctcttctctggtggcggtacatctggcctgtcaaagtttaatcaacggcgaatcggagttggcgatcgccgccggggtgaatttgattttgacccccgatgtgacccagacttttacccaggcgggcatgatgagtaagacgggccgttgccagacctttgatgccgaggctgatggctatgtgcggggcgaaggttgtggggtcgttctcctcaaacccctggcccaggcagaacgggacggggataatattctcgcggtgatccacggttcggcggtgaatcaagatggacgcagtaacggtttgacggctcccaacgggcgatcgcaacaggccgttattcgccaagccctggcccaagccggcattaccgccgccgatttagcttacctagaggcccacggcaccggcacgcccctgggtgatcccattgaaattaattccctgaaggcggttttacaaacggcgcagcgggaacagccctgtgtggtgggttctgtgaaaacaaacattggtcacctcgaggcagcggcgggcatcgcgggcttaatcaaggtgattttgtccctagagcatggaatgattccccaacatttgcattttaagcagctcaatccccgcattgatctagacggtttagtgaccattgcgagcaaagatcagccttggtcaggcgggtcacaaaaacggtttgctggggtaagttcctttgggtttggtggcaccaatgcccacgtgattgtcggggactatgctcaacaaaaatctccccttgctcctccggctacccaagaccgcccttggcatttgctgaccctttctgctaaaaatgcccaggccttaaatgccctgcaaaaaagctatggagactatctggcccaacatcccagcgttgacccacgcgatctctgtttgtctgccaataccgggcgatcgcccctcaaagaacgtcgtttttttgtctttaaacaagtcgccgatttacaacaaactctcaatcaagattttctggcccaaccacgcctcagttcccccgcaaaaattgcctttttgtttacggggcaaggttcccaatactacggcatggggcaacaactgtaccaaaccagcccagtatttcggcaagtgctggatgagtgcgatcgcctctggcagacctattcccccgaagcccctgccctcaccgacctgctgtacggtaaccataaccctgacctcgtccacgaaactgtctatacccagcccctcctctttgctgttgaatatgcgatcgcccaactatggttaagctggggcgtgacgccagacttttgcatgggccatagcgtcggcgaatatgtcgcggcttgtctggcgggggtattttccctggcagacggcatgaaattaattacggcccggggcaaactgatgcacgccctacccagcaatggcagtatggcggcggtctttgccgataaaacggtcatcaaaccctacctatcggagcatttgaccgtcggagccgaaaacggttcccatttggtgctatcaggaaagaccccctgcctcgaagccagtattcacaaactccaaagccaagggatcaaaaccaaacccctcaaggtttcccatgctttccactcccctttgatggctcccatgctggcagagtttcgggaaattgctgaacaaattactttccacccgccgcgtatcccgctcatttccaatgtcacgggcggccagattgaagcggaaattgcccaggccgactattgggttaagcacgtttcgcaacccgtcaaatttgtccagagcatccaaaccctggcccaagcgggtgtcaatgtttatctcgaaatcggcgtaaaaccagtgctcctgagtatgggacgccattgcttagctgaacaagaagcggtttggttgcccagtttacgtccccatagtgagccttggccggaaattttgaccagtctcggcaaactgtatgagcaagggctaaacattgactggcagaccgtggaagctggcgatcgccgccggaaactgattctgcccacctatcccttccaacggcaacgatattggtttaatcaaggctcttggcaaactgttgagaccgaatctgtgaacccaggccctgacgatctcaatgattggttgtatcaggtggcgtggacgcccctggacactttgcccccggcccctgaaccgtcggctaagctgtggttaatcttgggcgatcgccatgatcaccagcccattgaagcccaatttaaaaacgcccagcgggtgtatctcggccaaagcaatcattttccgacgaatgccccctgggaagtatctgccgatgcgttggataatttatttactcacgtcggctcccaaaatttagcaggcatcctttacctgtgtcccccaggggaagacccagaagacctagatgaaattcaaaagcaaaccagtggcttcgccctccaactgatccaaaccctgtatcaacaaaagatcgcggttccctgctggtttgtgacccaccagagccaacgggtgcttgaaaccgatgctgtcaccggatttgcccaagggggattatggggactcgcccaggcgatcgccctcgaacatccagagttgtgggggggaattattgatgtcgatgacagcctgccaaattttgcccagatttgccaacaaagacaggtgcagcagttggccgtgcggcaccaaaaactctacggggcacagctcaaaaagcaaccgtcactgccccagaaaaatctccagattcaaccccaacagacctatctagtgacagggggactgggggccattggccgtaaaattgcccaatggctagccgcagcaggagcagaaaaagtaattctcgtcagccggcgcgctccggcagcggatcagcagacgttaccgaccaatgcggtggtttatccttgcgatttagccgacgcagcccaggtggcaaagctgtttcaaacctatccccacatcaaaggaattttccatgcggcgggtaccttagctgatggtttgctgcaacaacaaacttggcaaaagttccagaccgtcgccgccgccaaaatgaaagggacatggcatctgcaccgccatagtcaaaagctcgatctggatttttttgtgttgttttcctctgtggcaggggtgctcggttcaccgggacaggggaattatgccgccgcaaaccggggcatggcggcgatcgcccaatatcgacaagcccaaggtttacccgccctggcgatccattgggggccttgggccgaagggggaatggccaactccctcagcaaccaaaatttagcgtggctgccgcccccccagggactaacaatcctcgaaaaagtcttgggcgcccagggggaaatgggggtctttaaaccggactggcaaaacctggccaaacagttccccgaatttgccaaaacccattactttgcagccgttattccctctgctgaggctgtgcccccaacggcttcaatttttgacaaattaatcaacctagaagcttctcagcgggctgactatctactggattatctgcggcggtctgtggcgcaaatcctcaagttagaaattgagcaaattcaaagccacgatagcctgttggatctgggcatggattcgttgatgatcatggaggcgatcgccagcctcaagcaggatttacaactgatgttgtaccccagggaaatctacgaacggcccagacttgatgtgttgacggcctatctagcggcggaattcaccaaggcccatgattctgaagcagcaacggcggcagcagcgattccctcccaaagcctttcggtcaaaacaaaaaaacagtggcaaaaacctgaccacaaaaacccgaatcccattgcctttatcctctctagcccccggtcgggttcgacgttgctgcgggtgatgttagccggacatccggggttatattcgccgccagagctgcatttgctcccctttgagactatgggcgatcgccaccaggaattgggtctatcccacctcggcgaagggttacaacgggccttaatggatctagaaaacctcaccccagaggcaagccaggcgaaggtcaaccaatgggtcaaagcgaatacacccattgcagacatctatgcctatctccaacggcaggcggaacaacgtttactcatcgacaaatctcccagctacggcagcgatcgccatattctagaccacagcgaaatcctctttgaccaggccaaatatatccatctggtacgccatccctacgcggtgattgaatcctttacccgactgcggatggataaactgctgggggccgagcagcagaacccctacgccctcgcggagtccatttggcgcaccagcaaccgcaatattttagacctgggtcgcacggttggtgcggatcgatatctccaggtgatttacgaagatctcgtccgtgacccccgcaaagttttgacaaatatttgtgatttcctgggggtggactttgacgaagcgctcctcaatccctacagcggcgatcgccttaccgatggcctccaccaacagtccatgggcgtcggggatcccaatttcctccagcacaaaaccattgatccggccctcgccgacaaatggcgctcaattaccctgcccgctgctctccagctggatacgatccagttggccgaaacgtttgcttacgatctcccccaggaaccccagctaacaccccagacccaatccttgccctcgatggtggagcggttcgtgacagtgcgcggtttagaaacctgtctctgtgagtggggcgatcgccaccaaccattggtgctacttctccacggcatcctcgaacagggggcctcctggcaactcatcgcgccccagttggcggcccagggctattgggttgtggccccagacctgcgtggtcacggcaaatccgcccatgcccagtcctacagcatgcttgattttttggctgacgtagatgcccttgccaaacaattaggcgatcgcccctttaccttggtgggccactccatgggttccatcatcggtgccatgtatgcaggaattcgccaaacccaggtagaaaagttgatcctcgttgaaaccattgtccccaacgacatcgacgacgctgaaaccggtaatcacctgacgacccatctcgattacctcgccgcgcccccccaacacccgatcttccccagcctagaagtggccgcccgtcgcctccgccaagccacgccccaactacccaaagacctctcggcgttcctcacccagcgcagcaccaaatccgtcgaaaaaggggtgcagtggcgttgggatgctttcctccgtacccgggcgggcattgaattcaatggcattagcagacgacgttacctggccctgctcaaagatatccaagcgccgatcaccctcatctatggcgatcagagtgaatttaaccgccctgctgatctccaggcgatccaagcggctctcccccaggcccaacgtttaacggttgctggcggccataacctccattttgagaatccccaggcgatcgcccaaattgtttatcaacaactccagacccctgtacccaaaacacaataa

SEQ ID NO: 5 - protein, 2720 aa, Synechococcus sp.
MVGQFANFVDLLQYRAKLQARKTVFSFLADGEAESAALTYGELDQKAQAIAAFLQANQAQGQRALLLYPPGLEFIGAFLGCLYAGVVAVPAYPPRPNKSFDRLHSIIQDAQAKFALTTTELKDKIADRLEALEGTDFHCLATDQVELISGKNWQKPNISGTDLAFLQYTSGSTGDPKGVMVSHHNLIHNSGLINQGFQDTEASMGVSWLPPYHDMGLIGGILQPIYVGATQILMPPVAFLQRPFRWLKAINDYRVSTSGAPNFAYDLCASQITPEQIRELDLSCWRLAFSGAEPIRAVTLENFAKTFATAGFQKSAFYPCYGMAETTLIVSGGNGRAQLPQEIIVSKQGIEANQVRPAQGTETTVTLVGSGEVIGDQIVKIVDPQALTECTVGEIGEVWVKGESVAQGYWQKPDLTQQQFQGNVGAETGFLRTGDLGFLQGGELYITGRLKDLLIIRGRNHYPQDIELTVEVAHPALRQGAGAAVSVDVNGEEQLVIVQEVERKYARKLNVAAVAQAIRGAIAAEHQLQPQAICFIKPGSIPKTSSGKIRRHACKAGFLDGSLAVVGEWQPSHQKEGKGIGTQAVTPSTTTSTNFPLPDQHQQQIEAWLKDNIAHRLGITPQQLDETEPFASYGLDSVQAVQVTADLEDWLGRKLDPTLAYDYPTIRTLAQFLVQGNQALEKIPQVPKIQGKEIAVVGLSCRFPQADNPEAFWELLRNGKDGVRPLKTRWATGEWGGFLEDIDQFEPQFFGISPREAEQMDPQQRLLLEVTWEALERANIPAESLRHSQTGVFVGISNSDYAQLQVRENNPINPYMGTGNAHSIAANRLSYFLDLRGVSLSIDTACSSSLVAVHLACQSLINGESELAIAAGVNLILTPDVTQTFTQAGMMSKTGRCQTFDAEADGYVRGEGCGVVLLKPLAQAERDGDNILAVIHGSAVNQDGRSNGLTAPNGRSQQAVIRQALAQAGITAADLAYLEAHGTGTPLGDPIEINSLKAVLQTAQREQPCVVGSVKTNIGHLEAAAGIAGLIKVILSLEHGMIPQHLHFKQLNPRIDLDGLVTIASKDQPWSGGSQKRFAGVSSFGFGGTNAHVIVGDYAQQKSPLAPPATQDRPWHLLTLSAKNAQALNALQKSYGDYLAQHPSVDPRDLCLSANTGRSPLKERRFFVFKQVADLQQTLNQDFLAQPRLSSPAKIAFLFTGQGSQYYGMGQQLYQTSPVFRQVLDECDRLWQTYSPEAPALTDLLYGNHNPDLVHETVYTQPLLFAVEYAIAQLWLSWGVTPDFCMGHSVGEYVAACLAGVFSLADGMKLITARGKLMHALPSNGSMAAVFADKTVIKPYLSEHLTVGAENGSHLVLSGKTPCLEASIHKLQSQGIKTKPLKVSHAFHSPLMAPMLAEFREIAEQITFHPPRIPLISNVTGGQIEAEIAQADYWVKHVSQPVKFVQSIQTLAQAGVNVYLEIGVKPVLLSMGRHCLAEQEAVWLPSLRPHSEPWPEILTSLGKLYEQGLNIDWQTVEAGDRRRKLILPTYPFQRQRYWFNQGSWQTVETESVNPGPDDLNDWLYQVAWTPLDTLPPAPEPSAKLWLILGDRHDHQPIEAQFKNAQRVYLGQSNHFPTNAPWEVSADALDNLFTHVGSQNLAGILYLCPPGEDPEDLDEIQKQTSGFALQLIQTLYQQKIAVPCWFVTHQSQRVLETDAVTGFAQGGLWGLAQAIALEHPELWGGIIDVDDSLPNFAQICQQRQVQQLAVRHQKLYGAQLKKQPSLPQKNLQIQPQQTYLVTGGLGAIGRKIAQWLAAAGAEKVILVSRRAPAADQQTLPTNAVVYPCDLADAAQVAKLFQTYPHIKGIFHAAGTLADGLLQQQTWQKFQTVAAAKMKGTWHLHRHSQKLDLDFFVLFSSVAGVLGSPGQGNYAAANRGMAAIAQYRQAQGLPALAIHWGPWAEGGMANSLSNQNLAWLPPPQGLTILEKVLGAQGEMGVFKPDWQNLAKQFPEFAKTHYFAAVIPSAEAVPPTASIFDKLINLEASQRADYLLDYLRRSVAQILKLEIEQIQSHDSLLDLGMDSLMIMEAIASLKQDLQLMLYPREIYERPRLDVLTAYLAAEFTKAHDSEAATAAAAIPSQSLSVKTKKQWQKPDHKNPNPIAFILSSPRSGSTLLRVMLAGHPGLYSPPELHLLPFETMGDRHQELGLSHLGEGLQRALMDLENLTPEASQAKVNQWVKANTPIADIYAYLQRQAEQRLLIDKSPSYGSDRHILDHSEILFDQAKYIHLVRHPYAVIESFTRLRMDKLLGAEQQNPYALAESIWRTSNRNILDLGRTVGADRYLQVIYEDLVRDPRKVLTNICDFLGVDFDEALLNPYSGDRLTDGLHQQSMGVGDPNFLQHKTIDPALADKWRSITLPAALQLDTIQLAETFAYDLPQEPQLTPQTQSLPSMVERFVTVRGLETCLCEWGDRHQPLVLLLHGILEQGASWQLIAPQLAAQGYWVVAPDLRGHGKSAHAQSYSMLDFLADVDALAKQLGDRPFTLVGHSMGSIIGAMYAGIRQTQVEKLILVETIVPNDIDDAETGNHLTTHLDYLAAPPQHPIFPSLEVAARRLRQATPQLPKDLSAFLTQRSTKSVEKGVQWRWDAFLRTRAGIEFNGISRRRYLALLKDIQAPITLIYGDQSEFNRPADLQAIQAALPQAQRLTVAGGHNLHFENPQAIAQIVYQQLQTPVPKTQ

SEQ ID NO: 6 - DNA, 951 bp, Synechococcus sp.
gtgcgcaaaccctggttagaacttcccttggcgattttttcctttggcttttataaagtcaacaaatttctgattgggaatctctacactttgtatttagcgctgaataaaaaaaatgctaaggaatggcgcattattggagaaaaatccctccagaaattcctgagtttacccgttttaatgaccaaagcgccccggtggaatacccacgccattatcggcaccctgggaccactctctgtagaaaaagaactcaccattaacctcgaaacgattcgtcaatccacggaagcttgggtcggttgcatctatgactttccgggctatcgcacggtgttaaatttcacgcaactcaccgatgaccccaaccaaacagaactcaaaattttcttacctaaagggaaatataccgtcgggttacgttactaccatcccaaggtaaatcctcgctttccggtcgttaaaacagatctaaatctaaccgtgccgactttggttgtttcgccccaaaacaacgacttttatcaagccctggcccagaaaacaaacctttattttcgtctgcttcactactacatttttacgctatttaaatttcgcgatgtcttacccgctgcttttgtgaaaggagaattcctccctgtcggcgccaccgatactcaatttttttacggcgctttagaagcagcagaaaacttagagattaccatcccagccccctggcttcagacctttgatttttatctcaccttctataaccgcgccagttttcccctacgttggcaaaaaatcaccgaagcgatgatctgtgatcccctgggagaaaaaggctattacctaattcggatgcggccccgtactcaggacgccgaggcacaattaccaacggttagaggagaagaaacccaggtcacgccccagcagaaaaaactggcgatccagtccctataa

SEQ ID NO: 7 - protein, 316 aa, Synechococcus sp.
MRKPWLELPLAIFSFGFYKVNKFLIGNLYTLYLALNKKNAKEWRIIGEKSLQKFLSLPVLMTKAPRWNTHAIIGTLGPLSVEKELTINLETIRQSTEAWVGCIYDFPGYRTVLNFTQLTDDPNQTELKIFLPKGKYTVGLRYYHPKVNPRFPVVKTDLNLTVPTLVVSPQNNDFYQALAQKTNLYFRLLHYYIFTLFKFRDVLPAAFVKGEFLPVGATDTQFFYGALEAAENLEITIPAPWLQTFDFYLTFYNRASFPLRWQKITEAMICDPLGEKGYYLIRMRPRTQDAEAQLPTVRGEETQVTPQQKKLAIQSL

SEQ ID NO: 8 - DNA, 984 bp, Cyanothece sp.
atgacccaaaaaacatcaacaatttttgaaatccccttggctttgttatccttcttattttacaaagccatgaaattcctcatcggcaatctttacacaatctatttaacttttaataaaagtaaagcctcacaatggcgagtcctatctgaagaagtcgtgatcaaaaccgccctcagcttaccggttttaatgacaaaaggtcctcgctggaatacccacgccatcatcggaacccttgggccctttaatgttaatcaatctattgctattgatttaaattcagctaatcaaactactcgatcctggatcgccgttatttatagttttccagggtatgaaactatcgcgagtcttgaatcaaatcgcattaaccctcaagaacaatgggcatctttagccttaaaacccggtaaatatagtatcggattgagatattataattggggtgaaaaagtgattgttccaacggttaaagtggatgatcagatatttgtagaatctcaatcgattccttcagatattaataagttttatttagatttaattcagaaaaaaaattggttttatttaagtcttcattattatatttttaccctgttgcggctgagaaagcggctaccagaatccttgataaaacaggaatatttaccggttggggcaacggatactgaatttgtctataattatttaacccgaggacaggcgctacaaatttctcttgattccgacttagttaagaattatgacatttacttgacaatttatgatcgttcgagtttaccgttaacttggagccaaattacagaagaaaactatttaacgaaacctatcgaaaacaacggctattatttaattcggatgcgccctaaatatgtctcgttagaagaagtgttaaaacagttaccggttcagtctgtaataagcgatgaagagacgttgactcaaaagcttaagctaaccgttaaaaccggtcaaaattaa

SEQ ID NO: 9 - protein, 327 aa, Cyanothece sp.
MTQKTSTIFEIPLALLSFLFYKAMKFLIGNLYTIYLTFNKSKASQWRVLSEEVVIKTALSLPVLMTKGPRWNTHAIIGTLGPFNVNQSIAIDLNSANQTTRSWIAVIYSFPGYETIASLESNRINPQEQWASLALKPGKYSIGLRYYNWGEKVIVPTVKVDDQIFVESQSIPSDINKFYLDLIQKKNWFYLSLHYYIFTLLRLRKRLPESLIKQEYLPVGATDTEFVYNYLTRGQALQISLDSDLVKNYDIYLTIYDRSSLPLTWSQITEENYLTKPIENNGYYLIRMRPKYVSLEEVLKQLPVQSVISDEETLTQKLKLTVKTGQN

SEQ ID NO: 10 - DNA, 990 bp, Cyanothece sp.
atgagtagtcaattttccaaattatctattgttgaactctttttagaattgcccttgactttgttatcttttgttttttacaaagtcatgaaatttatgattggcaatttatatacagtctatttaacctttaataaaagtaaaacatctcaatggcgagtcttatcagaagaggtaattaaatctgccctcagtgtaccggttttaatgactaaagggcctcgttggaatactcatgctattattggaacacttggccctttttccgttaatcaatctattgctattgatttaaattcagttaatcaaacctctcaatcttggattgccgttatttataactttccccaatatgaaaccattaccagtttagaatcaaaccgaattaattccgataatcaatgggcttgtttgaccttaaaaccggggaaatatagtataggattgagatattataactggggagaaaaggttgtttttccctcgataaaagttgaggataaagtttttgttgatcctcaagttatcccctcagaagtgaatcagttttattcgagtttaattaattataaaaactggttttatttaagtcttcattattatatttttaccctgttgagattgagaaaaattttgccagattcttttgtcaaacaggaatatttacccgttggggcaacggatacggaatttgtctataattatttactcaaagggcaagccttacaaattacccttgactcagaattagttaagaattatgacatttacttgacaatttatgatcggtctagtttgcccttaagttgggatcggatcatagaagacaagtatttaacaaaaccgatagaaaacaacggatattatttaattcggatgcggcctaaatatacctccttagaagaaatcttaacagagttaccagttgagtctcaaatcagtgatgaaaccgaattaattcaacagcttaaattaaaagttaaaggctaa

SEQ ID NO: 11 - protein, 329 aa, Cyanothece sp.
MSSQFSKLSIVELFLELPLTLLSFVFYKVMKFMIGNLYTVYLTFNKSKTSQWRVLSEEVIKSALSVPVLMTKGPRWNTHAIIGTLGPFSVNQSIAIDLNSVNQTSQSWIAVIYNFPQYETITSLESNRINSDNQWACLTLKPGKYSIGLRYYNWGEKVVFPSIKVEDKVFVDPQVIPSEVNQFYSSLINYKNWFYLSLHYYIFTLLRLRKILPDSFVKQEYLPVGATDTEFVYNYLLKGQALQITLDSELVKNYDIYLTIYDRSSLPLSWDRIIEDKYLTKPIENNGYYLIRMRPKYTSLEEILTELPVESQISDETELIQQLKLKVKG

SEQ ID NO: 12 - DNA, 846 bp, Lyngbya majuscule
atgcaaaccatcggaggatactttacctccaaaaaaaacactaaaaatctccagtggcaactcgtatcagccgagtttttaaaaaagcccatcaaattaatttgggcaatgagtcgagctcgttggaatcttcacgctattatttctctagttggaccgattcaggtcaaagagctaattagctttgatgccagtgcagctaaacaatcagcccaatcctggacattagtagtttacagtctaccagattttgaaaccatcactaatatcagctccctgaccgtatccggagaaaaccaatgggaatccgtgatcttaaaaccaggtaaatacttattaggtttgcggtattatcactggtcagagacagtagagcaacctactgttaaagcagatggtgttaaagtcgtagatgccaagcaaattcacgcccctactgatatcaacagcttttaccgtgacctaattaaacgaaaaaattggcttcatgtctggttaaattattatgtcttcaacctgttgcactttaagcaatggttaccccaggcatttgttaaaaaagtattcttacctgtaccgaatccagaaaccaaattttactatggtgccttgaaaaagggagaatcgattcaatttaaactagcaccatccttgttaacaagccatgatctttactacagcttgtacagccgtgaatgctttccgctagattggtacaaaattactgaaggggaacatagaacatctgctagtgagcagaagtctatttatattgttcggattcatccgaaatttgagcgaaacgctttatttgaaaatagttgggtgaagatagccgttgtttga

SEQ ID NO: 13 - protein, 281 aa, Lyngbya majuscule
MQTIGGYFTSKKNTKNLQWQLVSAEFLKKPIKLIWAMSRARWNLHAIISLVGPIQVKELISFDASAAKQSAQSWTLVVYSLPDFETITNISSLTVSGENQWESVILKPGKYLLGLRYYHWSETVEQPTVKADGVKVVDAKQIHAPTDINSFYRDLIKRKNWLHVWLNYYVFNLLHFKQWLPQAFVKKVFLPVPNPETKFYYGALKKGESIQFKLAPSLLTSHDLYYSLYSRECFPLDWYKITEGEHRTSASEQKSIYIVRIHPKFERNALFENSWVKIAVV

SEQ ID NO: 14 - DNA, 1011 bp, Lyngbya majuscule
atggaaactaaagaaaaatttttattcttccaactctggtgggaaattccactagcattgttatctttgatattttataaagctgttaagggacttatacccattctttttcaaaagaaaaccaaaaccaagaaaaaaatagcagacttaaccaaaaaagaagtttataaatggcgatttgtttctgaagaactgctaaaacagcctctggtactatcctatattttaactactggtcctcgatggaatgtccacgccattattgccactacagaaccggttccagtcaaagaatcattaaaaattgatatcagttcttgtgtggcttcagctcagtcatggagtataggtatctatagttttcctgaaggcaaacctgtcaaatacatagcatctcatgagccaaaatttcataaacaatggcaagaaatcaaactggaaccgggaaaatataatttagctttaagatattataattggtacgatcaagtcagtttacctgctgttattatggataataatcaaattatcaatactgaatcagttaatagtagtcagattaacaattacttcaattatttgcccaaattaataggacaagataatattttttatcgatttcttaattactatatattcactattctagtatgccagaaatggctacctaaagaatgggttagaaaagaatttttacctgtgggagaccccaataatgagtttgtctatggagttatttataaaggttactatttggctctgacattaaatccattattactcactaattatgatgtttatttaaccacatacaatcgttctagtctaccaattaatttttgtcaaattaatactgacaaatacacaacttctgtgatagaaaccgacggtttttatttagtgcgattgcgtcctaagtcagatttagacaataatttatttcagctaaattggattagtacagagcttgtatcagaagtttcctgtaaccgttcagggggcgaagtctga

SEQ ID NO: 15 - protein, 336 aa, Lyngbya majuscule
METKEKFLFFQLWWEIPLALLSLIFYKAVKGLIPILFQKKTKTKKKIADLTKKEVYKWRFVSEELLKQPLVLSYILTTGPRWNVHAIIATTEPVPVKESLKIDISSCVASAQSWSIGIYSFPEGKPVKYIASHEPKFHKQWQEIKLEPGKYNLALRYYNWYDQVSLPAVIMDNNQIINTESVNSSQINNYFNYLPKLIGQDNIFYRFLNYYIFTILVCQKWLPKEWVRKEFLPVGDPNNEFVYGVIYKGYYLALTLNPLLLTNYDVYLTTYNRSSLPINFCQINTDKYTTSVIETDGFYLVRLRPKSDLDNNLFQLNWISTELVSEVSCNRSGGEV

SEQ ID NO: 16 - DNA, 921 bp, Haliangium ochraceum
atgcgccgtagtcgtctgttgctcgaggcccccctcgcgctcgcctccttcgccctcaaccgcgcggccctggcgcgcgccctgaagccgatgagtcgcgcgcccgccagcgaccaaccgcgcgcgtggaagctcatggacgaggcgttctttgccccgccttcggtcatgacagcgtactcgctgctggcgccgcgatggaacgtgcacgcggccatcgcggtctcgccgattcttcccgtgaccggacgcgtgtccgtcgacgtcgccgctgccaacgcagcatccccgcgttggacgctcgtcgcctacgacaagcaagggacggtcgccgccgtcggcaccacaaacaccgaagcagacgcatcctgggccgccatcgagctgtcgcccggactgtatcgcttcgtgattcgcctctacgagcccgggcccggcggggtggtccccgaagtccatatcgatggcgagccggcgctcgccgcattggagctgccagaagacccgactcgtgtgtatcggagcctgcgcgcccgcggcgggcggaggcaccgagcgttgcagcgatacgtctatcccatggtgcggctgcggcggctcctcggcgaggagcgcgtgacccgcgagtacttaccggtgggaaaccccgagaccctgtttcgctttggcgtggtcgagcgcggtcagcggctcgaactccgcccgcccgacgaattacccgatgattgcggcctgtatctatgcctatacgatcagtcgagtctgcccatgtggttcgggccaatcctgcccgagggcatacagacgccgcctgcgccggaccacggcacctggctcgtccgcatcgtgcccgggcggcatggcgcgccggatccggcacggattcaggttcgcgtaatgtccgaaaagccgatcgcgtaa

SEQ ID NO: 17 - protein, 306 aa, Haliangium ochraceum
MRRSRLLLEAPLALASFALNRAALARALKPMSRAPASDQPRAWKLMDEAFFAPPSVMTAYSLLAPRWNVHAAIAVSPILPVTGRVSVDVAAANAASPRWTLVAYDKQGTVAAVGTTNTEADASWAAIELSPGLYRFVIRLYEPGPGGVVPEVHIDGEPALAALELPEDPTRVYRSLRARGGRRHRALQRYVYPMVRLRRLLGEERVTREYLPVGNPETLFRFGVVERGQRLELRPPDELPDDCGLYLCLYDQSSLPMWFGPILPEGIQTPPAPDHGTWLVRIVPGRHGAPDPARIQVRVMSEKPIA

SEQ ID NO: 18 - DNA, 1005 bp, Synechococcus sp.
atgcgcaaaccctggttagaacttcccttggcgattttttcctttggcttttataaagtcaacaaatttctgattgggaatctctacactttgtatttagcgctgaataaaaaaaatgctaaggaatggcgcattattggagaaaaatccctccagaaattcctgagtttacccgttttaatgaccaaagcgccccggtggaatacccacgccattatcggcaccctgggaccactctctgtagaaaaagaactcaccattaacctcgaaacgattcgtcaatccacggaagcttgggtcggttgcatctatgactttccgggctatcgcacggtgttaaatttcacgcaactcaccgatgaccccaaccaaacagaactcaaaattttcttacctaaagggaaatataccgtcgggttacgttactaccatcccaaggtaaatcctcgctttccggtcgttaaaacagatctaaatctaaccgtgccgactttggttgtttcgccccaaaacaacgacttttatcaagccctggcccagaaaacaaacctttattttcgtctgcttcactactacatttttacgctatttaaatttcgcgatgtcttacccgctgcttttgtgaaaggagaattcctccctgtcggcgccaccgatactcaatttttttacggcgctttagaagcagcagaaaacttagagattaccatcccagccccctggcttcagacctttgatttttatctcaccttctataaccgcgccagttttcccctacgttggcaaaaaatcaccgaagcgatgatctgtgatcccctgggagaaaaaggctattacctaattcggatgcggccccgtactcaggacgccgaggcacaattaccaacggttagaggagaagaaacccaggtcacgccccagcagaaaaaactggcgatccagtccctaggtttgcaccatcaccaccatcatagcgcctggagccacccgcagtttgaaaagtaa

SEQ ID NO: 19 - protein, 334 aa, Synechococcus sp.
MRKPWLELPLAIFSFGFYKVNKFLIGNLYTLYLALNKKNAKEWRIIGEKSLQKFLSLPVLMTKAPRWNTHAIIGTLGPLSVEKELTINLETIRQSTEAWVGCIYDFPGYRTVLNFTQLTDDPNQTELKIFLPKGKYTVGLRYYHPKVNPRFPVVKTDLNLTVPTLVVSPQNNDFYQALAQKTNLYFRLLHYYIFTLFKFRDVLPAAFVKGEFLPVGATDTQFFYGALEAAENLEITIPAPWLQTFDFYLTFYNRASFPLRWQKITEAMICDPLGEKGYYLIRMRPRTQDAEAQLPTVRGEETQVTPQQKKLAIQSLGLHHHHHHSAWSHPQFEK

SEQ ID NO: 20 - DNA, 161 bp, Artificial Sequence
  Description of Artificial Sequence Synthetic polynucleotide
atgatcaggaggagtcttttttgagtgctagctcccctgacgcagggtcactcttgtaagttccagtagcactcttttggcaagcattgaagcattcaaaccagtgaaatcccctcgctggagcagcgaagtttaagctatcgttgaagtagccaccttgg

SEQ ID NO: 21 - DNA, 278 bp, Artificial Sequence
  Description of Artificial Sequence Synthetic polynucleotide
tagtacaaaaagacgattaaccccatgggtaaaagcaggggagccactaaagttcacaggtttacaccgaattttccatttgaaaagtagtaaatcatacagaaaacaatcatgtaaaaattgaatactctaatggtttgatgtccgaaaaagtctagtttcttctattcttcgaccaaatctatggcagggcactatcacagagctggcttaataatttgggagaaatgggtgggggcggactttcgtagaacaatgtagattaaagtactgtacat

SEQ ID NO: 22 - DNA, 792 bp, Artificial Sequence
  Description of Artificial Sequence Synthetic polynucleotide
atgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataa

SEQ ID NO: 23 - DNA, 15559 bp, Artificial Sequence
  Description of Artificial Sequence Synthetic polynucleotide
ttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggcctgcaggacaactcggcttccgagcttggctccaccatggttatatctggagtaaccagaatttcgacaacttcgacgactatctcggtgcttttacctccaaccaacgcaaaaacattaagcgcgaacgcaaagccgttgacaaagcaggtttatccctcaagatgatgaccggggacgaaattcccgcccattacttcccactcatttatcgtttctatagcagcacctgcgacaaatttttttgggggagtaaatatctccggaaacccttttttgaaaccctagaatctacctatcgccatcgcgttgttctggccgccgcttacacgccagaagatgacaaacatcccgtcggtttatctttttgtatccgtaaagatgattatctttatggtcgttattggggggcctttgatgaatatgactgtctccattttgaagcctgctattacaaaccgatccaatgggcaatcgagcagggaattacgatgtacgatccgggcgctggcggaaaacataagcgacgacgtggtttcccggcaaccccaaactatagcctccaccgtttttatcaaccccgcatgggccaagttttagacgcttatattgatgaaattaatgccatggagcaacaggaaattgaagcgatcaatgcggatattccctttaaacggcaggaagttcaattgaaaatttcctagcttcactagccaaaagcgcgatcgcccaccgaccatcctcccttgggggagatgcggccgcgcgaaaaaaccccgccgaagcggggttttttgcggacgtcttacttttcaaactgcgggtggctccaggcgctatgatggtggtgatggtgcaaacctagggactggatcgccagttttttctgctggggcgtgacctgggtttcttctcctctaaccgttggtaattgtgcctcggcgtcctgagtacggggccgcatccgaattaggtaatagcctttttctcccaggggatcacagatcatcgcttcggtgattttttgccaacgtaggggaaaactggcgcggttatagaaggtgagataaaaatcaaaggtctgaagccagggggctgggatggtaatctctaagttttctgctgcttctaaagcgccgtaaaaaaattgagtatcggtggcgccgacagggaggaattctcctttcacaaaagcagcgggtaagacatcgcgaaatttaaatagcgtaaaaatgtagtagtgaagcagacgaaaataaaggtttgttttctgggccagggcttgataaaagtcgttgttttggggcgaaacaaccaaagtcggcacggttagatttagatctgttttaacgaccggaaagcgaggatttaccttgggatggtagtaacgtaacccgacggtatatttccctttaggtaagaaaattttgagttctgtttggttggggtcatcggtgagttgcgtgaaatttaacaccgtgcgatagcccggaaagtcatagatgcaaccgacccaagcttccgtggattgacgaatcgtttcgaggttaatggtgagttctttttctacagagagtggtcccagggtgccgataatggcgtgggtattccaccggggcgctttggtcattaaaacgggtaaactcaggaatttctggagggatttttctccaataatgcgccattccttagcattttttttattcagcgctaaatacaaagtgtagagattcccaatcagaaatttgttgactttataaaagccaaaggaaaaaatcgccaagggaagttctaaccagggtttgcgcatatgatcaggaggagtcttttttgagtgctagctcccctgacgcagggtcactcttgtaagttccagtagcactcttttggcaagcattgaagcattcaaaccagtgaaatcccctcgctggagcagcgaagtttaagctatcgttgaagtagccaccttggttaattaattggcgcgccgagcatctcttcgaagtattccaggcatcaaataaaacgaaaggctcagtcgaaagactgggcctttcgttttatctgttgtttgtcggtgaacgctctctactagagtcacactggctcaccttcgggtgggcctttctgcgtttataaagctttagtacaaaaagacgattaaccccatgggtaaaagcaggggagccactaaagttcacaggtttacaccgaattttccatttgaaaagtagtaaatcatacagaaaacaatcatgtaaaaattgaatactctaatggtttgatgtccgaaaaagtctagtttcttctattcttcgaccaaatctatggcagggcactatcacagagctggcttaataatttgggagaaatgggtgggggcggactttcgtagaacaatgtagattaaagtactgtacatatggcaagctggtcccacccgcaattcgagaaagaagtacatcaccatcaccatcatggcgcagtgggccagtttgcgaactttgtagacctgttgcaataccgtgccaagctgcaagcacgtaagaccgtctttagcttcctggcggacggcgaagcggagagcgccgctctgacctatggtgagctggatcaaaaggcgcaggcaatcgcggcgttcctgcaagcaaatcaggcacaaggccaacgtgcattgctgctgtatccgccaggtctggagttcatcggtgccttcctgggttgtctgtatgcgggtgtcgtcgcggttccggcatatcctccgcgtccgaacaagtccttcgaccgtttgcactccatcattcaggacgcccaagcgaagtttgcactgacgacgaccgagttgaaggataagattgcagaccgtctggaagcgctggagggtacggacttccattgcctggcgaccgaccaagtcgagctgatcagcggcaaaaactggcaaaagccgaatatctccggtacggatctggcgtttctgcaatacaccagcggcagcacgggtgatccaaaaggcgtgatggtcagccaccataacctgattcacaatagcggtctgattaaccagggtttccaagacaccgaagcgagcatgggtgtgtcctggctgccgccgtatcacgacatgggtctgattggcggcatcctgcaacctatctacgttggcgcaacgcaaatcctgatgccaccagtcgcctttctgcaacgtccgttccgctggctgaaggcgatcaacgattaccgtgtcagcaccagcggtgcgccgaactttgcttacgacctgtgcgcttctcagattaccccggaacaaatccgcgagctggatctgagctgttggcgtctggcattcagcggtgcagagccgattcgcgctgtcacgctggaaaactttgcgaaaacgttcgcaaccgcgggtttccagaaatcggccttctacccttgttacggtatggcggaaaccaccctgatcgtgagcggtggcaatggccgtgcccaactgccacaggagatcatcgttagcaagcagggcattgaggcgaaccaagtgcgtccggctcaaggcacggaaacgaccgtgaccctggtgggtagcggtgaggtcattggtgaccagatcgttaagatcgttgaccctcaagcgctgaccgagtgcaccgtcggtgaaattggcgaggtgtgggttaaaggtgaaagcgttgctcagggctactggcagaagccggacttgacgcagcagcagttccagggtaacgtgggtgccgaaacgggtttcctgcgcaccggcgatctgggtttcctgcaaggcggcgagctgtatatcaccggccgtctgaaggatctgctgatcattcgtggccgtaatcactatcctcaggacattgagctgaccgtggaagttgctcacccagccctgcgtcagggcgcaggtgccgcggtgagcgtggacgttaatggtgaagaacaactggtgatcgttcaagaggttgagcgtaagtacgcacgcaagctgaatgtggcagcagtcgctcaggccatccgtggtgcgattgcggcagagcaccagttgcagccgcaggcgatctgctttatcaaaccgggcagcatcccgaaaactagcagcggcaaaatccgtcgtcacgcatgtaaggccggttttctggacggaagcttggcggttgttggtgagtggcaaccgagccatcagaaagagggcaaaggtattggtacccaggcagtgaccccgagcaccacgacgtccaccaactttccgctgccggatcaacaccagcaacagatcgaggcgtggctgaaggacaacatcgcgcaccgcctgggtattacgccgcagcagttggatgaaacggaaccgttcgcttcttacggtctggacagcgttcaagcagtccaggtcaccgcagacctggaggactggctgggccgcaagctggacccgactctggcctatgattacccgaccattcgcacgctggcgcaattcctggttcagggcaaccaggccttggagaaaatcccgcaagttccaaagattcagggtaaagagattgcggtggtgggcctgagctgccgctttccgcaggcggacaatccggaggcgttctgggaactgttgcgcaatggcaaggatggcgtgcgtccgctgaaaacccgttgggccactggtgagtggggtggtttcctggaggatatcgaccagtttgagccgcagttctttggtattagcccgcgtgaggcggagcaaatggacccgcaacagcgtctgctgctggaggtcacctgggaggcactggagcgtgcgaatatccctgccgaatccctgcgtcacagccagaccggcgtctttgtgggcattagcaacagcgattacgcacaactgcaagtgcgtgagaacaacccgatcaatccgtacatgggtactggtaacgcacatagcatcgcggcgaatcgtctgagctactttctggatctgcgcggtgtctccctgagcattgataccgcgtgttctagcagcctggtcgcagttcatctggcgtgccaaagcctgattaacggcgagagcgagctggcgattgctgcgggtgttaatctgattctgaccccggatgtcacgcaaacctttacccaagcgggtatgatgagcaagacgggccgttgccagacgtttgatgcggaggcggacggctacgtgcgcggtgaaggctgcggcgttgttctgctgaaaccgctggctcaggcggagcgtgatggcgacaatatcctggcggtcatccacggtagcgcggttaaccaggacggtcgcagcaatggtctgactgcgccgaacggccgctctcagcaagcggttatccgtcaggccctggcgcaggcgggcatcaccgcggcagacctggcgtatttggaagcgcatggtacgggcaccccgctgggcgacccgattgaaatcaacagcttgaaagcagtgctgcaaaccgcccagcgcgagcaaccgtgcgttgtgggcagcgtcaagacgaacattggccacctggaggcagcagcgggtattgcaggtctgatcaaggtgattctgtccctggagcacggcatgattccgcaacacctgcactttaagcaactgaatccgcgcatcgacctggacggcctggttaccatcgcgagcaaagaccagccgtggtcgggtggtagccagaagcgtttcgccggtgtcagcagctttggttttggcggtacgaatgctcacgtgattgttggtgattatgcccagcaaaagtccccgctggctccgcctgcgacccaagaccgtccttggcatctgctgactctgagcgcgaagaacgcacaagcgttgaacgcgttgcaaaagagctatggtgactacctggcgcaacatccgagcgttgaccctcgcgatctgtgcctgagcgctaacactggtcgctctccgctgaaagaacgccgcttcttcgtgttcaagcaggttgccgacttgcaacaaaccctgaatcaggactttctggcgcagccgaggctgagcagcccagccaagattgcgttcctgttcacgggtcagggcagccagtactacggtatgggccagcaactgtatcagacgtccccggttttccgtcaagtcctggatgaatgcgaccgtctgtggcagacgtacagcccggaggcaccggcgctgaccgatctgctgtacggcaatcataatcctgacctggttcatgaaacggtttacacgcaaccgctgctgttcgcggtggagtatgctatcgcgcagttgtggttgagctggggcgttactccggatttctgcatgggtcatagcgtcggtgagtatgtggcggcctgcctggcgggtgtgtttagcctggcggatggcatgaaactgattaccgcgcgtggtaaactgatgcatgcactgccgagcaatggcagcatggcggctgtgtttgcggacaaaaccgttatcaagccgtatctgagcgaacacctgaccgtcggcgcagaaaatggcagccacctggttctgagcggtaagaccccttgtctggaagcatccatccacaaactgcaaagccagggcatcaaaaccaagcctctgaaagtctcccatgcgttccactcgccgctgatggcgccgatgctggcggaatttcgtgagatcgccgaacagattacgttccatccgccacgtatcccgctgattagcaacgtgacgggtggtcaaatcgaggccgagatcgcgcaagcagactattgggttaaacatgttagccagccggtgaagttcgttcagagcattcagaccctggcccaagcgggtgtgaatgtgtacctggaaatcggtgttaaaccagtcctgctgtctatgggtcgccactgtctggcagagcaggaagcggtttggctgccgagcctgcgtccacatagcgagccttggccggaaatcttgactagtctgggcaaactgtacgagcaaggtctgaatatcgactggcaaacggttgaagccggtgatcgccgtcgtaagctgattttgccgacctacccgttccagcgtcagcgttattggttcaaccaaggtagctggcaaaccgtcgaaactgagagcgtgaatccaggcccggacgacctgaatgactggctgtaccaagtggcatggactccgctggatacgctgccgcctgcaccggaaccgtcggcgaaactgtggctgattctgggtgatcgtcacgatcaccaaccgattgaggcccagttcaaaaacgcccaacgtgtgtacctgggccaaagcaaccactttccgacgaacgccccgtgggaggtgagcgcggacgcactggataacttgtttacccatgtgggtagccaaaacctggcaggcattctgtatctgtgcccgcctggtgaagatccggaggatctggatgagattcagaaacaaacttccggctttgcgttgcaactgattcagaccctgtatcagcagaaaatcgcagtgccgtgttggtttgttacccatcaaagccagcgtgtgctggaaacggacgcggtgacgggttttgcccaaggtggtctgtggggtttggcgcaagcgattgcactggaacatccggaactgtggggtggtatcattgacgtggatgatagcctgccgaacttcgcgcagatttgtcagcaacgtcaggttcagcaactggctgtccgtcaccagaaactgtatggtgcgcaactgaagaagcagccgagcctgccgcagaagaatctgcagatccaacctcaacagacctacctggtcacgggcggtttgggtgcaatcggtcgtaagattgcgcagtggctggcggctgcgggtgctgagaaagttatcctggttagccgtcgtgcaccggcagcggatcaacaaaccttgccgaccaacgccgtggtgtacccgtgcgatctggcggatgcggcgcaggttgcgaaactgttccaaacctatccgcacattaagggtatctttcatgcagccggtacgctggctgacggtttgctgcaacagcaaacctggcagaaattccagactgtcgctgcggcgaagatgaagggcacctggcacctgcatcgccactctcagaagttggacttggatttctttgttttgttttcgtctgttgcgggtgtgctgggtagccctggtcaaggcaattacgcggcagccaaccgtggcatggccgccatcgctcagtaccgccaggctcaaggtctgccggcactggcgattcactggggcccttgggcggaaggtggtatggcaaacagcttgagcaaccaaaatctggcatggttgcctccgccgcagggcttgaccattctggaaaaagttttgggtgcccaaggcgaaatgggcgtgttcaaaccggactggcagaacttggccaaacaattcccggagttcgcgaaaacccattactttgcggcggtcattccgagcgctgaagcggttccaccgaccgcatctatcttcgacaagctgatcaatctggaagcgagccagcgcgcagattacctgctggactatctgcgtagatctgtggcacaaattctgaaactggaaattgagcagattcagagccacgactccctgctggatctgggtatggatagcctgatgatcatggaggcgattgcgtccctgaaacaagacctgcaactgatgctgtatccgcgtgagatttacgagcgtccgcgtctggatgttctgactgcttacttggccgctgagtttaccaaagcgcatgattctgaagcagctaccgccgcagctgcgatccctagccagagcctgagcgtcaaaaccaaaaagcaatggcagaaaccggatcataagaacccgaatccgattgcgttcatcctgagcagcccgcgtagcggtagcaccctgctgcgcgtgatgctggccggtcacccgggtctgtattccccaccggaactgcacctgctgccgtttgaaacgatgggtgaccgccaccaggaactgggtctgtctcatctgggcgagggtctgcaacgtgccctgatggacttggaaaatctgacgccggaagcatcccaggcaaaggtgaaccaatgggtgaaggcgaatacgccgattgcagacatctacgcatacctgcaacgtcaagccgagcaacgtctgctgattgacaaaagcccgagctatggcagcgaccgccacattctggatcacagcgagatcctgttcgatcaggcgaaatacatccacctggttcgccatccttatgcggtcattgagagctttacccgcctgcgtatggacaagctgctgggtgcagagcaacagaatccgtatgcgctggcggaaagcatttggcgtacctcgaatcgcaacattctggacttgggtcgtaccgtcggcgctgaccgctacctgcaagtcatctacgaggatctggtgcgtgacccgcgtaaagttctgaccaacatttgtgattttctgggtgtcgatttcgacgaggcactgctgaatccgtactccggcgaccgcctgaccgacggcctgcaccagcaaagcatgggtgtgggtgacccgaacttcttgcagcacaagaccattgatccggcgctagcggacaaatggcgtagcattaccctgccggctgctctgcaactggatacgattcaactggccgaaaccttcgcatacgacctgccgcaggagccgcagttgacgccgcagacccaatctttgccatcgatggtcgaacgtttcgtcacggttcgcggcctggaaacctgtctgtgcgagtggggtgatcgccatcaacctctggtcttgctgttgcacggtatcctggagcaaggcgcgtcttggcagttgatcgcgcctcaactggcagcgcagggctattgggtcgtcgctccggatctgcgcggtcacggtaaatctgcgcacgcgcagtcttatagcatgctggattttctggccgatgtggacgcgctggccaaacagttgggcgaccgtccgttcaccttggttggtcacagcatgggttccatcattggcgcaatgtatgctggcattcgtcaaacccaggttgaaaaactgattctggtcgaaaccatcgtcccgaatgatattgatgatgccgaaaccggcaatcacctgaccacccatctggattacctggcagcccctccgcagcacccgatctttccgagcctggaagttgcggctcgtcgtctgcgccaagccaccccgcagttgccgaaagacctgtctgcatttctgacgcaacgttccacgaagagcgtcgagaagggtgtgcagtggcgctgggatgccttcttgcgcacccgtgcaggtatcgagtttaacggtatcagccgtcgccgttatctggcgctgctgaaagatatccaggccccaattactttgatttacggtgatcagtctgagttcaatcgcccagcagacctgcaagcgatccaggcggcactgccgcaagcgcaacgcctgacggttgctggcggtcacaacttgcactttgagaatccgcaggccatcgcccagattgtctatcagcagttgcagacaccggttccgaaaacccaaggtttgcaccatcaccaccatcatagcgcctggagccacccgcagtttgaaaagtaaggatccctctatatcagaattcggttttccgtcctgtcttgattttcaagcaaacaatgcctccgatttctaatcggaggcatttgtttttgtttattgcaaaaacaaaaaatattgttacaaatttttacaggctattaagcctaccgtcataaataatttgccatttactagtttttaattaaccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaggcgcgcctgatcagttggtgctgcattagctaagaaggtcaggagatattattcgacatctagctgacggccattgcgatcataaacgaggatatcccactggccattttcagcggcttcaaaggcaattttagacccatcagcactaatggttggattacgcacttcttggtttaagttatcggttaaattccgcttttgttcaaactcgcgatcatagagataaatatcagattcgccgcgacgattgaccgcaaagacaatgtagcgaccatcttcagaaacggcaggatgggaggcaatttcatttagggtattgaggcccggtaacagaatcgtttgcctggtgctggtatcaaatagatagatatcctgggaaccattgcggtctgaggcaaaaacgaggtagggttcggcgatcgccgggtcaaattcgagggcccgactatttaaactgcggccaccgggatcaacgggaaaattgacaatgcgcggataaccaacgcagctctggagcagcaaaccgaggctaccgaggaaaaaactgcgtagaaaagaaacatagcgcataggtcaaagggaaatcaaagggcgggcgatcgccaatttttctataatattgtcctaacagcacactaaaacagagccatgctagcaaaaatttggagtgccaccattgtcggggtcgatgccctcagggtcggggtggaagtggatatttccggcggcttaccgaaaatgatggtggtcggactgcggccggccaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgctt

SEQ ID NO: 24 - DNA, 42 bp, Artificial Sequence
  Description of Artificial Sequence Synthetic primer
gggagctcaaggaattatagttatgcgcaaaccctggttaga

SEQ ID NO: 25 - DNA, 42 bp, Artificial Sequence
  Description of Artificial Sequence Synthetic primer
ggcctgcaggttatagggactggatcgccagttttttctgct